(19) United States
(12) Patent Application Publication
(10) Pub. No.: US 2022/0145320 A1
(43) Pub. Date: May 12, 2022

(54) GENETICALLY MODIFIED PLANTS WITH ENHANCED DROUGHT TOLERANCE

(71) Applicant: Rothamsted Research Limited, Hertfordshire (GB)

(72) Inventors: Jonathan Napier, Hertfordshire (GB); Smita KURUP, Hertfordshire (GB); Michaela Matthes, Hertfordshire (GB)

(21) Appl. No.: 17/439,106

(22) PCT Filed: Mar. 13, 2020
(86) PCT No.: PCT/GB2020/050639

§ 371 Date: Sep. 14, 2021

(30) Foreign Application Priority Data
Mar. 14, 2019 (GB) 1903485.9
Apr. 25, 2019 (GB) 1905793.4

(51) Int. Cl. C12N 15/82

(52) U.S. Cl. CPC C12N 15/8273; C12N 15/8269; C12N 15/8225

(57) ABSTRACT

This invention relates to genetically modified plants with improved drought tolerance and to genetically modified cells thereof. These plants may have thicker leaves, rounder leaf shape or higher chlorophyll content. The plants may be modified to increase the expression of a plant calmodulin-binding protein IQD22, IQD23 or IQD24. The modified plants are expected to have improved yields under variable field conditions. The invention also relates to a method of genetically modifying a plant to have these characteristics. The invention further relates to isolated nucleic acids and polynucleotide constructs for the genetic engineering of such plants and their use.

This invention relates to plants with improved drought tolerance. The plants are genetically modified plants. The invention therefore also concerns isolated nucleic acids and polynucleotide constructs used in the genetic engineering of such plants. The modified plant materials and germplasm have potential application in the generation and breeding of new more drought resistant plants, including modification of existing plant varieties to improve field performance.

### BACKGROUND

Water scarcity is among the main problems to be faced by many societies and the world in the 21st century. Freshwater is a limited and dwindling global resource, but water use has been growing at more than twice the rate of population increase in the last century, and, although there is no global water scarcity as such, an increasing number of regions are chronically short of water (http://www.un.org/waterforlifedecade/scarcity.shtml).

Water-limited conditions impose abiotic stresses such as dehydration and salinity on crops which limit their performance and reduce yield worldwide. Therefore, it becomes socially and economically increasingly important to achieve greater crop production under water deficient conditions.

With the aim to generate better performing plants under water scarcity, two strategies are usually pursued: marker assisted breeding programs or the more targeted approach of genetic engineering. Microarray and DNA chip technologies and more recently the advent of next-generation sequencing technology RNA-seq have identified numerous drought-responsive transcripts. Many of the genes identified in this way have been used for the generation of transgenic plants in order to test their effects on improving drought resistance in crops by overexpression and/or suppression. These genes can be classified into several groups targeting different aspects of cell function such as signalling (regulation of gene expression by protein kinases and transcription factors), protein degradation and/or modification, metabolism of phytohormones, osmotic adjustment, synthesis of protective proteins, energy regulation and signalling, transport proteins of diverse functions, amino acid metabolism and proteins involved in reactive oxygen species scavenging (see Hu, H. and Xiong L., (2014) Annual Review Plant Biology Vol. 65: 715-741, and references therein)

However, what has not been addressed in transgenic approaches hitherto is a modification of traits of plant architecture which could confer an increased survival rate in water-limited conditions. These are inherently complex traits with the contribution of many loci and are therefore difficult to target by genetic engineering. One such trait which has been shown to give an advantage to plants grown in water-limited conditions are leaves with a higher LMA (leaf dry mass per unit area in g·m⁻²) which is frequently used as a proxy for leaf thickness. Leaf thickness, the distance between upper (adaxial) and lower (abaxial) leaf surface, has been shown to correlate with environmental variables such as water availability, temperature and light quality and on a global scale, across habitats and land plant diversity, plants adapted to arid environments tend to have thicker leaves (see Poort, H., et al. (2009) New Phytologist 182: 565-588).

In their work on leaf thickness of the domesticated tomato Solanum lycopersicum and the desert adapted S. penneffii, Coneva, V. et al., (2017) Plant Physiology Vol 175: 376-391 attempted to elucidate the genetic mechanisms responsible for leaf thickness and identified several QTLs for this trait. In addition, they showed that increased elongation of the palisade cells was the main contributor to leaf thickness. Overexpression of the HRD gene, an AP2/ERF-like transcription factor, in Arabidopsis thaliana led to an increase in number of leaf mesophyll cells and leaf thickness and conferred drought resistance and salt tolerance to the plants (Karaba et al., (2007) Proc. Natl. Acad. Sci. USA Vol 25; 104(39):15270-15275). This increased drought resistance could be replicated in rice upon transfer of Arabidopsis HRD to rice.

Although leaf thickness is a highly functional trait, mechanistic understanding of its development during leaf ontogeny is poorly understood. This is confounded by the problem that besides the innate genetic component responsible for leaf thickness there is a prominent role for environmental cues in modulating it. Currently available data obtained in Arabidopsis thaliana suggest that increased thickness of the leaf is the result of palisade cell elongation and the formation of additional palisade cell layers and that this morphological change is regulated by perception of blue light of lower intensities through the phototropin receptor PHOT2 (Kozuka, T. et al. (2011) Plant Cell Vol 23(10): 3684-3695). On the downside however, leaf thickness is negatively correlated with yield-related traits which points towards a trade-off between investment in vegetative and reproductive biomass.

Disclosed in Steffen, A. et al., (2005) BMC Evolutionary Biology 5: 72 doc10.11186/1471-2148-5-72 is a genome-wide comparative analysis of the IQD gene families in Arabidopsis thaliana and Oryza sativa. 33 IQD1-like genes in Arabidopsis thaliana and 29 genes in Oryza sativa were identified and analyzed. The encoded IQD proteins contain a plant-specific domain of 67 conserved amino acid residues, referred to as the IQ67 domain, which is characterized by a unique and repetitive arrangement of three different calmodulin recruitment motifs, known as the IQ, 1-5-10 and 1-8-14 motifs. An IQD-like gene in bryophytes suggests that IQD proteins are an ancient family of calmodulin-binding proteins and arose during the early evolution of land plants. Comparative phylogenetic analyses indicate that the major IQD gene lineages originated before the monocot-eudicot divergence.

Disclosed in Bürstenbinder, K. et al. (2017) Plant Pysiol. 173: 1692-1708 is a scientific publication showing how the 33 members of the IQD family in Arabidopsis thaliana differentially localize, using green fluorescent protein (GFP)-tagged proteins, to multiple and distinct subcellular sites, including microtubule (MT) arrays, plasma membrane subdomains, and nuclear compartments. The various IQD-specific localization patterns coincide with the subcellular patterns of IQD-dependent recruitment of calmodulin, suggesting that the diverse IQD members sequester calcium-calmodulin signaling modules to specific subcellular sites for precise regulation of Ca2+-dependent processes. GFP-labeled microtubule arrays were analyzed quantitatively in Nicotiana benthamiana cells transiently expressing GFP-IQD fusions. IQD-specific microtubule patterns were observed which point to a role of IQDs in MT organization and dynamics. Stable overexpression of select IQD proteins in Arabidopsis altered cellular microtubule orientation, cell shape, and organ morphology. The authors suggest that IQD families provide an assortment of platform proteins for integrating calmodulin-dependent calcium signaling at multiple cellular sites to regulate cell function, shape and growth.

To date therefore, the IQD proteins are characterised to some small degree at the level of cell structure and biochemistry. So far, only IQD1 has been assigned a putative role in glucosinolate metabolism in Arabidopsis (see Levy M et al, Plant J. 2005 July; 43(1):79-96).

Xiao, H. et al., (2008) Science Vol 319(5869): 1527-1530 describes the function of the an IQD gene known as the SUN gene in tomato. Altered expression of this gene is observed to be linked to changes in tomato fruit shape.

The problem therefore remains as to how to modify plant architecture of plants in a reliable enough way so as to improve the drought tolerance of such modified plants, and yet further ideally without diminution of plant growth. In the context of agricultural species and varieties, the improvement of drought tolerance ideally needs to go hand-in-hand with little or no significant diminution of yield of harvestable product.

The inventors have unexpectedly discovered that when engineering Arabidopsis thaliana and Camelina sativa plants to overexpress IQD genes, not only is there an increased leaf thickness compared to unmodified control plants, but also an increased thickness of the stem. There is a surprisingly favourable tolerance to drought in the overexpressing plants.

### BRIEF SUMMARY OF THE DISCLOSURE

In accordance with the present invention there is provided a method of modifying a plant, whereby at least some of the leaves of the modified plant are: (i) thicker; and/or (ii) rounder in shape; and/or (iii) contain more chlorophyll per wet or dry weight, compared to a corresponding unmodified plant, comprising genetically modifying the plant to increase expression of a plant calmodulin-binding protein (IQD) compared to the unmodified plant grown under same or comparative conditions.

Alternatively, in accordance with the present invention is provided a method of modifying a plant to increase yield compared to a corresponding unmodified plant, comprising genetically modifying the plant so as to increase expression of a plant calmodulin-binding protein (IQD) compared to the unmodified plant grown under same or comparative conditions.

Therefore the invention provides for the use of a polynucleotide comprising a nucleic acid sequence encoding a plant calmodulin-binding protein (IQD) for increasing the expressed amount of an IQD protein or polypeptide in plant cells modified with the polynucleotide, compared to a corresponding unmodified plant cell, and wherein in a plant of such modified cells at least some of the leaves of the plant are: (i) thicker; and/or (ii) rounder in shape; and/or (iii) contain more chlorophyll per wet or dry weight, compared to a corresponding plant of unmodified cells.

The plant calmodulin-binding protein (IQD) may be a nuclear and/or a microtubule associated protein. The plant calmodulin binding protein in being associated with microtubules may be bound thereto.

In any of the aforementioned methods and uses of the invention, the IQD protein is selected from one or more of IQD22, IQD23 and IQD24. Sequence information for these IQD proteins is set forth hereinafter.

The IQD protein for use in accordance with the invention preferably comprises a conserved 67 amino acid domain (IQ67) having an amino acid sequence with at least 50% identity to FRGYLARRALRALKGLVRLQALVRG [SEQ ID NO: 10] or at least 50% identity to:

[SEQ ID NO: 11]
EE#AA# + IQX#FRGYLARRALRALKGLVRLQALVRGX#VR + QA##T
L + CMQALVR#QAXVRARR# + #,

wherein “#” is a hydrophobic amino acid, and wherein “+” is a basic amino acid.

The aforementioned SEQ ID NO: 11 is referred to in Steffen, A. et al., (2005) BMC Evolutionary Biology 5: 72 doc10.11186/1471-2148-5-72. Steffen et al (2005) also refers to various motifs (1-20) of IQD proteins. Reference to numbered motifs of IQD proteins in this specification correspond to the numbered motifs described by Steffen et al (2005).

Additionally or alternatively, the polypeptide may further comprise the amino acid sequence:

[SEQ ID NO: 15]
(i) HAIAVAAATAAVAEAA;

or

(ii) (D/R/S/N)(K/Q/D/N/R/T)(H/R)(A/S)(I/V/M/S)(A/H)V(A/T)(A/E/F)(A/P)T(A/E)(A/H/V)(V/A) [SEQ ID NO: 16]; preferably DKHAIAVAAATAAV [SEQ ID NO: 17]. These are amino acid sequences located as at motifs 7 and/or 8 or portion thereof, of IQD proteins. The sequence DKHAIAVAAATAAV [SEQ ID NO: 18] represents an amino acid sequence portion determined by the inventors to be in common to and unique for all IQD 22, IQD 23 and IQD 24 proteins.

Additionally to the above of motif 7, the polynucleotide may further comprise an amino acid sequence portion of motif 8 being:

[SEQ ID NO: 19]
V(A/Q)(E/N/D/K)(A/N)(A/R)(V/Q/I/L)(A/T/N)(A/I)A(Q/
E/V/N/H/R/N)AAA(A/V/E)VV(R/K/E/S/L/T)(L/F)(T/N)X,
wherein X is any amino acid; more preferably
[SEQ ID NO: 20]
V(A/Q)(E/N/D/K)(A/N)(A/R)(V/Q/I/L)(A/T/N)(A/I)A(Q/
E/V/N/H/R/N)AAA(A/V/E)VVRLTX
or
[SEQ ID NO: 21]
V(A/Q)(E/N/D/K)(A/N)(A/R)(V/Q/I/L)(A/T/N)(A/I)A(Q/
E/V/N/H/R/N)AAA(A/V/E)VVRLTS
or
[SEQ ID NO: 22]
V(A/Q)(E/N/D/K)(A/N)(A/R)(V/Q/I/L)(A/T/N)(A/I)A(Q/
E/V/N/H/R/N)AAAEVVRLTX.

Additionally or alternatively to any of the above, the polypeptide may further comprise an amino acid sequence of about motif 5 being: (R/K)(R/K/T)W(S/G)F [SEQ ID NO: 33] or (R/K)(R/K/T)WSF [SEQ ID NO: 34]; preferably wherein this sequence is RRWSF[SEQ ID NO: 35] or RKWSF [SEQ ID NO: 36]. The sequence portion RRWSF represents an amino acid sequence determined by the inventors to be in common to and unique for all IQD 22, IQD 23 and IQD 24 proteins.

In any of the aforementioned aspects, the polynucleotide may comprise an amino acid sequence of motif 17 being:

[SEQ ID NO: 23]
G(Y/D)HP(N/S)YMA(N/C)TES(Y/S)(K/R)(V/A)RS(Q/A)SAP
(K/R)(Q/S)R.

Additionally or alternatively, the polynucleotide may further comprise a C-terminal portion amino acid sequence being:

[SEQ ID NO: 24]
L(H/Y)SATSRSKRSAFTASSIAPSDCT(Q/K)SCC(Y/D)(A/G)DHPS
YMACTESSRAKARSAPKSRPQL(Y/F)YE(Q/R)(S/P)SSKRFG(Y/F)
VD(L/V)PYCGD(S/T)(R/K)SGPQK(V/G)SALHTSFMNKAYPGSGRL
DRLGMPIGYRY.

In any of the aforementioned aspects, the polypeptide may have:

(1) an amino acid sequence of SEQ ID NO: 1 (IQD 22) or an amino acid sequence of at least 30% identity therewith;

(2) an amino acid sequence of SEQ ID NO: 4 (IQD 23) or an amino acid sequence of at least 30% identity therewith;

(3) an amino acid sequence of SEQ ID NO: 7 (IQD 24) or an amino acid sequence of at least 30% identity therewith.

In second separate aspect, the invention provides the use of a polynucleotide encoding a polypeptide having the amino acid sequence of SEQ ID NO: 1 (IQD 22) or an amino acid sequence of at least 30% identity therewith, for increasing (i) leaf thickness; and/or (ii) roundness of leaf shape; and/or (iii) chlorophyll content per wet or dry weight of leaf in a genetically modified plant and when compared to a corresponding unmodified plant grown under same or similar conditions, wherein the expression of the polypeptide in a modified plant is increased compared to a corresponding unmodified plant.

In other words, the invention includes the use of a polynucleotide encoding a polypeptide having the amino acid sequence of SEQ ID NO: 1 (IQD 22) or an amino acid sequence of at least 30% identity therewith, for increasing the expressed amount of the polypeptide in a plant cell modified with the polynucleotide, compared to a corresponding unmodified plant cell, and wherein in a plant of such modified cells at least some of the leaves of the plant are: (i) thicker; and/or (ii) rounder in shape; and/or (iii) contain more chlorophyll per wet or dry weight, compared to a corresponding unmodified plant; and/or the stem(s) of the plant are thicker compared to the stem(s) of a corresponding unmodified plant. In third separate aspect, the invention provides the use of a polynucleotide encoding a polypeptide having an amino acid sequence of SEQ ID NO: 4 (IQD 23) or an amino acid sequence of at least 30% identity therewith, for increasing (i) leaf thickness; and/or (ii) roundness of leaf shape; and/or (iii) chlorophyll content per wet or dry weight of leaf in a genetically modified plant and when compared to a corresponding unmodified plant grown under same or similar conditions, wherein the expression of the polypeptide in a modified plant compared to a corresponding unmodified plant.

In other words, the invention includes the use of a polynucleotide encoding a polypeptide having an amino acid sequence of SEQ ID NO: 4 (IQD 23) or an amino acid sequence of at least 30% identity therewith, for increasing the expressed amount of the polypeptide in a plant cell modified with the polynucleotide, compared to a corresponding unmodified plant cell; and wherein in a plant of such modified cells at least some of the leaves of the plant are: (i) thicker; and/or (ii) rounder in shape; and/or (iii) contain more chlorophyll per wet or dry weight, compared to a corresponding unmodified plant; and/or the stem(s) of the plant are thicker compared to the stem(s) of a corresponding unmodified plant.

In fourth separate aspect, the invention provides the use of a polynucleotide encoding a polypeptide having an amino acid sequence of SEQ ID NO: 7 (IQD 24) or an amino acid sequence of at least 30% identity therewith, for increasing (i) leaf thickness; and/or (ii) roundness of leaf shape; and/or (iii) chlorophyll content per wet or dry weight of leaf in a genetically modified plant and when compared to a corresponding unmodified plant grown under same or similar conditions, wherein the expression of the polypeptide in a modified plant compared to a corresponding unmodified plant cell.

In other words, the invention includes the use of a polynucleotide encoding a polypeptide having an amino acid sequence of SEQ ID NO: 7 (IQD 24) or an amino acid sequence of at least 30% identity therewith, for increasing the expressed amount of the polypeptide in a plant cell modified with the polynucleotide, compared to a corresponding unmodified plant cell, and wherein in a plant of such modified cells at least some of the leaves of the plant are: (i) thicker; and/or (ii) rounder in shape; and/or (iii) contain more chlorophyll per wet or dry weight, compared to a corresponding unmodified plant; and/or the stem(s) of the plant are thicker compared to the stem(s) of a corresponding unmodified plant.

In any of the second, third or fourth aspects of the invention as herein defined, the amino acid sequence of the polypeptide may include one or more of the following amino acid sequences:

[SEQ ID NO: 25]
a) (X/K)RRWSF, wherein X is any amino acid;
[SEQ ID NO: 26]
b) DKHAIAVAAATAAVAEAA;
[SEQ ID NO: 27]
c) VVRLT(X/S), wherein X is any amino acid;
[SEQ ID NO: 28]
d) KIQS;
[SEQ ID NO: 29]
e) FRGYLA;
[SEQ ID NO: 30]
f) RALRALK;
[SEQ ID NO: 31]
g) ENSPQ(X/L), wherein X is any amino acid;
[SEQ ID NO: 32]
h) VRGHI.

Alternatively, in any of the second, third or fourth aspects of the invention, the amino acid sequence of the polypeptide may include one or more of the following amino acid sequences:

[SEQ ID NO: 25]
a) (X/K)RRWSF, wherein X is any amino acid;
[SEQ ID NO: 37]
b) DKHAIAVAAATAAVAEAA(X/L), wherein X is any amino
acid;
[SEQ ID NO: 27]
c) VVRLT(X/S), wherein X is any amino acid;
[SEQ ID NO: 28]
d) KIQS;
[SEQ ID NO: 29]
e) FRGYLA;
[SEQ ID NO: 30]
f) RALRALK;
[SEQ ID NO: 31]
g) ENSPQ(X/L), wherein X is any amino acid;
[SEQ ID NO: 32]
h) VRGHI;
[SEQ ID NO: 38]
i) MGFFGRLFGSKK;
[SEQ ID NO: 39]
j) AAAEVVRLT;
[SEQ ID NO: 40]
k) RSNRRW;
[SEQ ID NO: 41]
l) AAMKIQSAFRGYLARRALRALKALVKLQALV;
[SEQ ID NO: 42]
m) GHIVRKQTADMLRRMQTLVRLQ;
[SEQ ID NO: 43]
n) ARARASRSSH;
[SEQ ID NO: 44]
o) DKILEVDTWKPH;
[SEQ ID NO: 45]
p) ESPRKR;
[SEQ ID NO: 46]
q) TPFTP;
[SEQ ID NO: 47]
r) YYSGYHPNYMANTESYKAKVRSQSAP;
[SEQ ID NO: 48]
s) SGYKRS;
[SEQ ID NO: 49]
t) QGQYYYYT.

In yet further alternative of the second, third or fourth aspects of the invention the polypeptide includes one or more of the following amino acid sequences:

a)
[SEQ ID NO: 38]
MGFFGRLFGSKK;
b)
[SEQ ID NO: 37]
DKHAIAVAAATAAVAEAA(X/L),;
wherein X is any amino acid
c)
[SEQ ID NO: 39]
AAAEVVRLT
or
[SEQ ID NO: 50]
XXXXVVRLT;
wherein X is any amino acid
d)
[SEQ ID NO: 51]
RSNRRWXX
or
[SEQ ID NO: 52]
XXXRRWSF;
wherein X is any amino acid
e)
[SEQ ID NO: 28]
KIQS
or
[SEQ ID NO: 41]
AAMKIQSAFRGYLARRALRALKALVKLQALV;
f)
[SEQ ID NO: 42]
GHIVRKQTADMLRRMQTLVRLQ;
g)
[SEQ ID NO: 43]
ARARASRSSH;
h)
[SEQ ID NO: 44]
DKILEVDTWKPH;
i)
[SEQ ID NO: 45]
ESPRKR;
j)
[SEQ ID NO: 46]
TPFTP;
k)
[SEQ ID NO: 47]
YYSGYHPNYMANTESYKAKVRSQSAP;
l)
[SEQ ID NO: 48]
SGYKRS;
m)
[SEQ ID NO: 49]
QGQYYYYT;
n)
[SEQ ID NO: 29]
FRGYLA;
o)
[SEQ ID NO: 30]
RALRALK;
p)
[SEQ ID NO: 53]
ENSPQL.

The invention includes an isolated polynucleotide comprising a nucleotide sequence encoding an IQD polypeptide as described herein. In preferred aspect the nucleotide sequence is that of (a) IQD 22 as shown in FIG. 44 or a sequence of at least 45% identity thereto; optionally at least 48% identity thereto; or (b) IQD 23 as shown in FIG. 44 or a sequence of at least 48% identity thereto; optionally at least 62% identity thereto; or (c) IQD 24 as shown in FIG. 44 or a sequence of at least 45% identity thereto; optionally at least 60% identity thereto; or (d) consensus sequence as shown in FIG. 44 or a sequence of at least 40% identity thereto; optionally at least 60% identity thereto.

The invention also includes an isolated cDNA molecule comprising or consisting of a nucleic acid sequence complementary to any sequence of FIG. 44 or variants thereof as referred to above.

Included in the invention is any polynucleotide construct for engineering, replication or expression, wherein the construct comprises a polynucleotide as described above. Expression constructs which may be integrated into the host cell genome at a locus other than the native IQD protein promoter will also comprise an heterologous promoter, e.g. a CMV promoter; optionally regulatory control elements such as an enhancer.

The invention also provides a genetically modified plant cell having an increased amount of an IQD polypeptide as hereinbefore defined, when compared to a corresponding unmodified plant cell.

The invention includes a genetically modified plant cell having at least one polynucleotide sequence encoding an IQD polypeptide as hereinbefore defined in addition to any naturally occurring homolog(s) of said protein(s) in a corresponding unmodified plant cell.

Genetically modified plant cells may also be defined as transgenic. Such transgenic plant cells may be modified such that they contain heterologous genetic material stably; optionally heritably, incorporated. The incorporation may be genomic, or it may be transient, e.g. extrachromosomal such as an expression plasmid.

The increased amount of polypeptide in modified plant cells may be at least 2-fold compared to the corresponding unmodified plant cell. In alternatives, the increased amount of polypeptide may be any of at least 3-fold, at least 4-fold, at least 5-fold, at least 6-fold, at least 7-fold (i.e. a log 2-fold), at least 8-fold, at least 9-fold, at least 10-fold, or at least 20-fold, when compared to a corresponding unmodified plant cell. The -fold increase may correspond to the number of additional IQD gene copies which are incorporated into the modified plant cell when compared to the corresponding unmodified cell. In connection with this, each copy of the heterologous IQD gene which is present where there is a multiplicity, may be same or different. Where copies are different then their sequences may be obtained or derived from different plant species.

Where genetically modified plant cells are provided in accordance with the invention then where there is at least one additional IQD polynucleotide sequence is under the control of a native promoter for that polypeptide

In a preferred aspect, the present invention provides a genetically modified plant cell as herein defined, wherein the polynucleotide sequence is as set forth herein for IQD 22 overexpression. These embodiments of the invention are advantageous in providing the ability to regenerate and provide plants wherein the leaves of the plants have taller (i.e. more elongated) palisade leaf cells compared to a corresponding unmodified plant. Additionally or alternatively, such IQD 22 overexpression advantageously causes an increased photosynthetic activity in the leaves of plant; such photosynthetic activity increase may be at least 5% more compared to corresponding unmodified control plants subjected to and measured under the same conditions. The increase may be 6% or more, 7% or more, 8% or more, 9% or more, 10% or more, 11% or more, 12% or more, 13% or more, 14% or more; or 15% or more.

In some embodiments, a genetically modified plant cell of the invention may have at least one additional IQD polynucleotide sequence under the control of an heterologous promoter; optionally a constitutive promoter, such as CMV S35. Inducible promoters may be used in case it is desired to switch IQD gene overexpression on and/or off during plant growth and development.

Where there is a multiplicity of additional IQD polynucleotide sequences introduced in tot the plant cell, then they may be incorporated at the same genetic locus, or possibly into different loci.

The invention therefore includes a plant comprising or consisting of genetically modified plant cells as hereinbefore described, wherein at least some of the leaves of the plant are (i) rounder in shape; and/or (ii) thicker; and/or (iii) contain more chlorophyll per wet or dry weight, compared to a corresponding unmodified plant. In particular, when using an heterologous promoter, e.g. S35, the leaves are about 1.8-2 times thicker. In such embodiments, the leaves may be about 1.5, about 1.6, about 1.7, about 1.8, about 1.9, about 2, about 2.1, about 2.2, about 2.3, about 2.4, about 2.5 or about 3 time thicker.

In certain plants, preferably when expressed from a native IQD gene promoter, at least some leaves are thicker by at least 25% on a leaf mass (g) per unit area (m⁻²) basis. Other plants may have thicker leaves by at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 45%, at least 50%.

In plants with thicker leaves as described herein, this may be due to at least some leaves having a palisade layer comprising more cells and/or more elongated cells compared to a corresponding unmodified plant. As already noted, in a preferred aspect of the invention, overexpression of IQD 22 results in a modified plant with leaves comprising more elongate palisade layer cells and/or a greater level of photosynthetic efficiency, compared to leaves of an unmodified control plant.

In any of the aforementioned plants of the invention there may be an elimination of epinastic leaf curling as found in a corresponding unmodified control plant kept under same conditions for growth and development.

Additionally or alternatively in any of the aforementioned plants of the invention the stem of the plant may be thicker compared a corresponding unmodified control plant kept under same conditions for growth and development.

Where leaves of a modified plant of the invention are concerned, they are advantageously more like leaves of succulent plants in character. They tend to exhibit a reduced transpiration rate compared to an unmodified control plant kept under the same conditions. Advantageously, the IQD gene overexpressing plants of the invention may have an altered physiology, wherein wilting is delayed when water is withheld compared to unmodified plant kept under the same conditions. Further, when a wilted plant is subjected to re-watering, a wilted plant of the invention recovers more quickly compared to an unmodified plant kept under the same conditions. In some instances, the unmodified plant does not recover whilst the modified plant of the invention does revive.

Further advantages of the modified plants of the invention are found. One is that yield of the plant may be increased under drought conditions compared to an unmodified control plant kept under the same drought conditions; e.g. wherein the yield is one or more of total biomass, organ or part biomass, leaf biomass, yield of fruit or yield of seed. For example, the yield may be 2-fold compared to the corresponding unmodified plant cell. In alternatives, the increased yield of seed may be any of at least 3-fold, at least 4-fold, at least 5-fold, at least 10-fold, at least 15-fold, at least 20-fold, at least 25-fold, at least 30-fold, at least 35-fold, at least 40-fold, at least 45-fold or at least 50-fold, when compared to a corresponding unmodified plant cell.

Consequently, the invention includes any plant part, plant tissue, organ, haploid or diploid reproductive material, cell callus, fruit, seed or organ obtained from a modified plant of the invention as hereinbefore described.

The invention additionally includes a method of increasing tolerance of a plant to water stress, comprising modifying the plant so that it expresses an increased amount of an IQD polypeptide as herein defined, at least in the leaves and/or stems of the plant compared to an unmodified control plant under the same growth conditions.

The invention further includes a method of increasing leaf thickness in a plant, comprising modifying the plant so that it expresses an increased amount of an IQD polypeptide as herein defined, at least in the leaves of the plant compared to an unmodified control plant.

The invention yet further provides a method of increasing stem thickness in a plant, comprising modifying the plant so that it expresses an increased amount of an IQD polypeptide as herein described, at least in the stems of the plant compared to an unmodified control plant subjected to the same conditions for growth and development.

The invention therefore also includes any processed material of a modified plant, plant part, plant tissue, organ, cell callus, fruit, seed or organ of the invention as described herein, wherein the processed material contains on a weight basis an increased amount of an IQD polypeptide and/or of an IQD polynucleotide as defined herein, when compared to a corresponding processed material from a control unmodified plant, plant part, plant tissue, organ, cell callus, fruit, seed or organ. Such processed plant material may be selected from meal, flour, oil, biomass for energy generation, wood or wood products.

In another aspect, the invention provides an isolated polynucleotide comprising a native IQD22 gene promoter and a gene of interest (GOI) intended for expression under the control of the promoter in a plant, plant tissue or plant cell. Such polynucleotides may further include a termination sequence, e.g. nos.

The polynucleotides herein described form part of a polynucleotide expression construct. When used to transform and produce transgenic plants, the expression of a GOI driven by the native IQD22 promoter results in leaf palisade cell specific expression of the GOI, when compared to a WT, i.e. untransformed plant. The invention therefore includes such transgenic plants and transgenic plant leaves which have leaf palisade specific expression of the GOI. Examples of GOI include genes involved in photosynthesis or gene involved in resistance to foliar pathogens.

The invention also provides an IQD22 gene promoter for use in an expression construct as a leaf palisade cell specific promoter.

The IQD22 gene promoter region is set forth in SEQ ID NO: 127. The IQD22 promoter is therefore all or a functional portion of this sequence. The IQD22 promoter sequence may be a sequence of SEQ ID NO: 127 or a sequence of at least 200 contiguous nucleotides thereof, starting at any position of SEQ ID NO: 127 that provides for at least 200 contiguous nucleotides. The promoter sequence of at least 200 contiguous nucleotides may have an identity of at least 60% to the corresponding aligned nucleotides of SEQ ID NO: 127. In preferred aspects, the at least 200 contiguous nucleotides of the promoter sequence may have at least 61%, at least 62%, at least 63%, at least 64%, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 127.

The invention will now be described in more detail and by way of specific examples and with reference to the drawings in which:

### BRIEF DESCRIPTION OF THE DRAWINGS

FIG. 1 shows the construct used transformation of Arabidopsis col-0 with IQD22 in order to generate IQD22 OE Arabidopsis plants.

FIG. 2 shows how plants of WT and IQD22 OE Arabidopsis line Fat5 compare.

FIG. 3 compares the rosette phenotype of WT and Fat1 and Fat5 OE Arabidopsis plants; (a) showing rosette size of WT and IQD22 OE lines after 33 days of growth, and (b) after 49 days.

FIG. 4a is a Northern blot of WT and IQD22 overexpressing Arabidopsis line.

FIG. 4b is a Southern blot comparing WT and OE lines.

FIG. 5A are bar charts of data for leaf width and leaf length comparing WT with Fat1 and Fat5 IQD22 OE Arabidopsis plants.

FIG. 5B is a bar chart showing data for average of dry matter (%) found in WT and Fat5 and Fat 6 OE Arabidopsis plants.

FIG. 6 is a series of micrographs (×350) of freeze fracture SEM Arabidopsis leaves from WT and Fat1.

FIG. 7 is a photograph comparing WT and IQD22 OE plants after 49 days growth. The OE Arabidopsis plants are less than half the height of the WT.

FIG. 8 are photomicrographs of stained stem sections from WT and Fat1 IQD22 OE Arabidopsis plants.

FIG. 9 is a photograph of all the leaves of WT compared to Fat1 and Fat5 IQD22 OE Arabidopsis plants.

FIG. 10 is a chart of comparing measured stem widths from WT and Fat 1 and Fat5 IQD22 OE Arabidopsis plants, wherein the stem is measured 1 cm about the rosette.

FIG. 11 are micrographs of stained longitudinal sections of interfascicular fibres of stems, comparing WT and IQD22 OE Arabidopsis plants.

FIG. 12 is a chart showing measurements comparing IQD22 OE and WT Arabidopsis plant leaf depths on transverse sections across the midpoint of the leaf lamina avoiding the midrib.

FIG. 13 are micrographs of leaf cross sections of WT and IQD22 OE under the native promoter; the Arabidopsis lines were grown under different light conditions.

FIG. 14 is a photograph of WT and Fat5 IQD22 OE Arabidopsis leaf discs; and a chart of the leaf mass per unit area determined for WT and OE leaves.

FIG. 15 is a series of micrographs showing the developmental time series of GUS expression controlled by the native IQD22 promoter in Arabidopsis.

FIG. 16 is a schematic representation of construct used for cellular localisation of IQD22.

FIG. 17 is a micrograph of cells expressing mCherry and showing how IQD22 is localised to the microtubules of palisade cells.

FIG. 18 shows (panel a) photographs of phenotypes of Arabidopsis lines expressing IQD22 under the control of its own promoter. Panel (b) shows epinastic leaf curling is abolished in the transgenic lines.

FIG. 19 shows the results of a time course experiment subjecting WT and IQD22 OE Arabidopsis plants to progressive drought.

FIG. 20 is a photograph of Arabidopsis WT and IQD22 OE lines under progressive drought over time.

FIG. 21 is a photograph of Arabidopsis WT and IQD22 OE plants, 11 days without water followed by 2 days of re-watering.

FIG. 22 is a time line and diagram showing when samples of Arabdopsis WT and IQD22 OE plants were taken for RNAseq analysis.

FIG. 23 is a PCA plot of RNAseq data obtained from WT and IQD22 OE Arabidopsis line fat1 subjected to progressive drought.

FIG. 24A shows SEM photomicrographs of abaxial leaf surfaces of WT and IQD22 OE Arabidopsis plants.

FIG. 24B shows SEM photomicrographs of flower buds of WT and IQD22 OE Arabidopsis plants.

FIG. 24C is a photograph of WT and IQD22 OE Arabidopsis seedlings grown on vertical agar plates.

FIG. 24D is a photomicrograph of roots of WT and IQD22 OE Arabidopsis seedlings stained with propidium iodide. Bar is 20 μm.

FIG. 24E is a photograph of WT and IQD22 OE Arabidopsis seedlings grown in compost.

FIG. 25A is a chart comparing stomatal conductance and photosynthetic rate of WT and IQD22 OE Arabidopsis lines.

FIG. 25B shows a chart (left hand panel) of stomatal density and stomatal index (right hand panel) for WT and IQD22 OE Arabidopsis lines.

FIG. 26 shows a rooted tree diagram of the IQD gene family of Arabidopsis.

FIG. 27 shows photographs of Arabidopsis WT and IQD23 overexpressing lines exposed to progressive drought.

FIG. 28 is a photograph showing silique length in WT and IQD22 OE Arabidopsis lines fat1 and fat5.

FIG. 29 is a chart showing number of seeds per silique of WT and IQD22 OE Arabidopsis lines.

FIG. 30 is a box plot of total seed yield obtained from 12 WT plants and 12 plants of IQD22 OE Arabidopsis lines grown under well-watered conditions.

FIG. 31 is a box plot of seed yield obtained from Arabidopsis, 12 WT plants and 12 plants of fat1 and fat5 IQD22 OE, respectively.

FIG. 32 is a schematic diagram of the reporter constructs used to localise IQD22 protein in Arabidopsis

FIG. 33 is a photograph of a Northern blot of RNA extracted from selected transformed Camelina lines and compared to wt

FIG. 34 shows photographs of transgenic Camelina illustrating morphological traits of the “strong” phenotype group. (a) shows the growth habit of “strong” line on the left compared to wt on the right. (b) shows leaf 13 and leaf 10 from a “strong” line (left) compared to wt (right). (c) shows the stem thickness of a strong transgenic line (top) as much increased compared to wt (bottom).

FIG. 35 is a micrograph of a freeze fracture through Camelina leaves of wt (left) and IQD22 overexpressing lines. The size of palisade cells is increased.

FIG. 36 is a photograph of transgenic and wt Camelina plants and their response to 4 days without watering. The wt shows signs of wilting.

FIG. 37 is a photograph showing wt and IQD22 overexpressing Camelina plants with a plant of the “strong” phenotype (middle) and intermediate phenotype (right) after 11 days after start of withholding water. The wilted wt is on the left.

FIG. 38 is a photograph showing wt and IQD22 overexpressing Camelina plants with a plant of the “strong” phenotype (middle) and intermediate phenotype (right) 11 days after rewatering. The wt is dead.

FIG. 39 is a graph showing the average seed yield per Camelina plant grown under an optimal watering regime. Line 35 is a strong IQD22 overexpressing plant, line 10_07 is an intermediate overexpressing plant.

FIG. 40 is a graph showing the average seed yield per Camelina plant after being subjected to 11 days of no watering, after which watering was resumed as normal. Line 35 is a strong IQD22 overexpressing plant, line 10_07 is an intermediate overexpressing plant.

FIG. 41 is a graph showing the average number of seeds per pod in well-watered transgenic lines of Camelina compared to wt.

FIG. 42 shows a sequence alignment of known sequences of motif 5 for IQD 22, IQD 23 and IQD 24 of various plant species.

FIG. 43 shows a sequence alignment of known sequences of motif 7 for IQD 22, IQD 23 and IQD 24 of various plant species.

FIG. 44 shows an alignment of nucleic acid sequences for each of IQD 22, IQD 23 and IQD 24.

FIG. 45 shows a GUS reporter gene construct employing the native IQD promoter which is used for transformation of Arabidopsis thaliana.

FIG. 46 is a micrograph of a section of leaf from Arabidopsis thaliana which has been transformed with the construct of FIG. 45.

FIG. 47 shows a construct for expression of IQD 22 in poplar under the control of a constitutive promoter.

FIG. 48 is a photograph showing a leaf of poplar transgenic clone M32 and a leaf of a normal WT poplar.

FIG. 49 is a scanning electron micrograph of a section of leaf from WT poplar.

FIG. 50 is a scanning electron micrograph of a section of leaf 14 from transgenic M32A poplar.

### DETAILED DESCRIPTION

Camelina is a genus within the flowering plant family Brassicaceae. Camelina is an emerging biofuel crop, in particular Camelina sativa. The plant is useful as a biofuel feedstock due in part to its drought tolerance and minimal requirements for supplemental nitrogen and other agricultural inputs (Gehringer et al. (2006) Genome 49(12): 1555-63; Gugel and Falk (2006) Canadian Journal of Plant Science 86(4): 1047-1058). C. sativa is of course in the same family and so relatively close genetically and developmentally and in many ways to Arabidopsis thaliana as well as the common oilseed crop Brassica napus (canola). The inventors were looking to further increase the drought tolerance of Camelina sativa in various ways, and in doing so discovered unexpectedly that if IQD genes are overexpressed in Arabidopsis or Camelina plants, then the plants have an altered morphology which and significantly improved drought tolerance compared to a corresponding unmodified control plant allowed to grow and develop under the same regime. More particularly the inventors have found that if IQD 22, IQD 23 or IQD 24 genes are overexpressed in modified Arabidopsis or Camelina plants, then the plants have an altered morphology which and significantly improved drought tolerance compared to an unmodified control plant. The morphology and architecture of the modified overexpressing plants is altered, manifest as a significant increase in the thickness of leaves and stem. This also affects biomass in the altered plants. There are also marked differences in physiology, that is to say in terms of transpiration and drought tolerance, and in certain instances in photosynthetic efficiency. There are also advantages gained in terms of seed yield and productivity.

Comparing the IQD 22 overexpressing Arabidopsis plants in particular with control (wt) plants under conditions of drought, wilting of the IQD 22 overexpressor lines is considerably delayed. Re-watering of droughted plants leads to a much quicker and better recovery of the IQD 22 overexpressors.

Naturally, an important industrial application of this invention is the engineering of more drought tolerant crops and the making available of such engineered germplasm for use in connection with the generation of new plant varieties. The inventors consider that the enhancements of drought tolerance in plants, particularly in preferred aspects, of the Brassicaceae, may be achieved by overexpression of IQD genes; preferably IQD 22 and/or IQD 23 and/or IQD 24 genes; more preferably IQD 22.

As used herein, the terms “increase”, “improve” or “enhance” are used interchangeably. Also, the terms “reduce” or “decrease” are used interchangeably.

As described herein, when the amino acid sequences of any IQD protein are defined herein with respect to a reference sequence, then homologues (i.e. sequence variants) are also defined with reference to a percentage of sequence identity. In the broadest aspect of the invention these variant sequences are at least 30% identical to a reference sequence. The proteins of the IQD family are unusual in that there is a very wide degree of variation between plant species. However, there are motifs, as described by Steffen, A. et al., (2005) Ibid. where there is much narrower degree of variation between plant species. The inventors define certain consensus sequences of IQD 22, 23 and 24 or combinations therefore and which may be used in combination with the reference sequence and at least 30% identity variants to further define the IQD polypeptides which are overexpressed in accordance with the invention.

Amino acid motifs which are intended to assist in defining IQD 22 and/or IQD 23 and/or IQD24 polypeptide sequences, whether or not together with SEQ ID NO: 1 or SEQ ID NO:4 or SEQ ID NO: 7, are as follows: RRWSF [SEQ ID NO: 35]; DKHAIAVAAATAAVAEAA [SEQ ID NO: 26]; WRLT; KIQS; FRGYLA [SEQ ID NO: 29]; RALRALK [SEQ ID NO: 30] or ENSPQ [SEQ ID NO: 54], whether alone or in any combination.

Amino acid motifs which are intended to assist in defining IQD22 and/or IQD 23 polypeptide sequences, whether or not together with SEQ ID NO: 1 or SEQ ID NO:4, are as follows: KRRWSF; VVRLTS or VRGHI, whether alone or in any combination.

Amino acid motifs which are intended to assist in defining IQD23 and/or IQD 24 polypeptide sequences, whether or not together with SEQ ID NO: 4 or SEQ ID NO:7, are as follows: MGFFGRLFGSKK [SEQ ID NO: 38]; DKHAIAVAAATAAVAEAAL [SEQ ID NO: 56]; AAAEVVRLT [SEQ ID NO: 39]; RSNRRW [SEQ ID NO: 40]; AAMKIQSAFRGYLARRALRALKALVKLQALV [SEQ ID NO: 41]; GHIVRKQTADMLRRMQTLVRLQ [SEQ ID NO: 42]; ARARASRSSH [SEQ ID NO: 43]; DKILEVDTWKPH [SEQ ID NO: 44]; ESPRKR [SEQ ID NO: 45]; TPFTP [SEQ ID NO: 46]; YYSGYHPNYMANTESYKAKVRSQSAP [SEQ ID NO: 47]; SGYKRS [SEQ ID NO: 48]; or QGQYYYYT [SEQ ID NO: 49].

Amino acid motifs which are intended to assist in defining IQD22 and/or IQD 24 polypeptide sequences are as follows: RRWSF [SEQ ID NO: 35]; VVRLT [SEQ ID NO: 55]; FRGYLA [SEQ ID NO: 29]; RALRALK [SEQ ID NO: 30] or ENSPQL [SEQ ID NO: 53].

Accordingly, the invention—having regard to any reference IQD sequence, i.e. SEQ ID NO: 1, SEQ ID NO: 4 or SEQ ID NO: 7—also includes any variant of such reference sequence which is at least 31%, at least 32%, at least 33%, at least 34%, at least 35%, at least 36%, at least 37%, at least 38%, at least 39%, at least 40%, at least 41%, at least at least 42%, at least 43%, at least 44%, at least 45%, at least 46%, at least 47%, at least at least 48%, at least 49%, at least 50%, at least 51%, at least 52%, at least 53%, at least at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity thereto. Sequence identity may be determined using a global alignment algorithm known in the art, such as the Needleman Wunsch algorithm in the program GAP (GCG Wisconsin Package, Accelrys). Other methods of sequence comparison and analysis may be used; e.g Bestfit (Wisconsin Sequence Analysis Package, Version 8 for Unix, Genetics Computer Group, University Research Park, 575 Science Drive Madison, Wis. 53711).

When using a sequence alignment program to determine whether a particular sequence has for instance 95% identity with a reference sequence of the present invention, the parameters are preferably adjusted so that the percentage of identity is calculated over the entire length of the reference sequence and homology gaps of up to 5% of the total number of the nucleotides in the reference sequence are permitted. Usually these may be default settings in commonly used software packages. Preferably therefore when a sequence has a percent identity to any one of the SEQ ID NOs as detailed herein, this refers to a sequence which has the stated percent identity over the entire length of the SEQ ID NO referred to.

The degree of sequence identity of polynucleotides of the invention may, instead of being expressed as a percentage identity to reference sequence, may instead be defined in terms of hybridization to a polynucleotide of reference sequence. Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

When employed in the invention herein, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated herein by reference.

Where the invention may provide a transgenic plant, the nucleic acids used in the method of the invention are not at their natural locus in the genome of said plant, it being possible for the nucleic acids to be expressed homologously or heterologously. Thus, the plant expresses a transgene. However, as mentioned, in certain embodiments, transgenic may means that, while the nucleic acids according to the different embodiments of the invention are at their natural position in the genome of a plant, the sequence has been modified with regard to the natural sequence, and/or that the regulatory sequences of the natural sequences have been modified, for example by mutagenesis.

Transgenic is preferably understood as meaning the expression of the nucleic acids according to the invention at an unnatural locus in the genome, i.e. homologous or, preferably, heterologous expression of the nucleic acids takes place. According to the invention, the transgene is stably integrated into the plant and the plant is preferably homozygous for the transgene.

Modified plant cells, modified plants or parts thereof as defined herein may be stably transformed with additional genetic material. Such additional genetic material is preferably under the control of at least one regulatory sequence, but a multiplicity of control points may be built in, whether using native or modified regulatory sequences.

Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

Transformation methods are well known in the art. Thus, according to the various aspects of the invention, an heterologous nucleic acid is introduced into a plant and expressed as a transgene. The nucleic acid sequence is introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner well known in the art.

To select transformed plants, plant material obtained in the transformation is, as a rule, subjected to selective conditions so that transformed plants can be distinguished from untransformed plants. For example, seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known in the art.

The generated transformed plants may be propagated by a variety of means, such as by clonal propagation or classical breeding techniques. For example, a first generation (or T₁) transformed plant may be selfed and homozygous second-generation (or T₂) transformants selected, and the T₂ plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The regulatory sequence may be a promoter; optionally an inducible promoter, preferably then one which may be induced by an external stress condition. In the alternative, a constitutive promoter may be employed, e.g. cauliflower mosaic 35S.

The regulatory sequence may optionally be tissue specific. The term “regulatory element” as used herein may be considered interchangeably with “control sequence” and “promoter” and all terms are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. Also possible is that the activity of the promoters is increased by modification of their sequence, or that they are replaced completely by more active promoters, even promoters from heterologous organisms. For expression in plants, the nucleic acid molecule is, as described above, preferably linked operably to or comprises a suitable promoter which expresses the gene at the right point in time and with the required spatial expression pattern. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

The term “operably linked” as used herein refers to a functional linkage between the promoter sequence and the gene of interest, such that the promoter sequence is able to initiate transcription of the gene of interest.

For example, the nucleic acid sequence may be expressed using a promoter that drives overexpression. Overexpression according to the invention means that the transgene is expressed at a level that is higher than expression of endogenous counterparts driven by their endogenous promoters. For example, overexpression may be carried out using a strong promoter, such as a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression. Alternatively, enhanced or increased expression can be achieved by using transcription or translation enhancers or activators and may incorporate enhancers into the gene to further increase expression. Furthermore, an inducible expression system may be used, where expression is driven by a promoter induced by environmental stress conditions (for example the pepper pathogen-induced membrane protein gene CaPIMPI or promoters that comprise the dehydration-responsive element (DRE), the promoter of the sunflower HD-Zip protein genes Hahbl or Hahb4, which is inducible by water stress, high salt concentrations and ABA or a chemically inducible promoter (such as steroid- or ethanol-inducible promoter system). The promoter may also be tissue-specific. The types of promoters listed above are described in the art. Other suitable promoters and inducible systems are also known to a person of average skill.

A green tissue-specific promoter may be used. For example, a green tissue-specific promoter may be selected from the maize orthophosphate kinase promoter, maize phosphoenolpyruvate carboxylase promoter, rice phosphoenolpyruvate carboxylase promoter, rice small subunit rubisco promoter, rice beta expansin EXBO9 promoter, pigeonpea small subunit rubisco promoter or pea RBS3A promoter.

The promoter may be a constitutive or strong promoter. In a preferred embodiment, the regulatory sequence is an inducible promoter or a stress inducible promoter. The stress inducible promoter is selected from the following non limiting list: the HaHB1 promoter, RD29A (which drives drought inducible expression of DREB1A), the maize rabl7 drought-inducible promoter, P5CS1 (which drives drought inducible expression of the proline biosynthetic enzyme P5CS1), ABA- and drought-inducible promoters of Arabidopsis clade A PP2Cs (ABM, ABI2, HAB1, PP2CA, HA11, HAI2 and HAI3) or their corresponding crop orthologues.

Altered plants in accordance with the invention advantageously may provide better yield characteristics. These may be designed into the alterations being made. Yield characteristics, also known as yield traits may comprise one or more of the following non-limitative list of features: biomass, seed yield, seed/grain size, starch content of grain, greenness index, increased growth rate. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square metres. The term “yield” of a plant may relate to vegetative biomass (root and/or shoot biomass), to reproductive organs, and/or to propagules (such as seeds) of that plant. Thus, according to the invention, yield comprises one or more of and can be measured by assessing one or more of: increased seed yield per plant, increased seed filling rate, increased number of filled seeds, increased harvest index, increased viability/germination efficiency, increased number or size of seeds/capsules/pods, increased growth, increased biomass or grain fill. Preferably, increased yield comprises an increased number of grains/seeds/capsules/pods, increased biomass, increased growth. Yield is usually measured relative to a control plant.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest. When referred to herein, a “plant part” may be green tissue, for example a leaf.

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

A dicot plant may be selected from the families including, but not limited to Asteraceae, Brassicaceae (e.g. Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine, bell pepper, chilli or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant)

Other useful and preferred plants are maize, rice, wheat, oilseed rape/canola, sorghum, soybean, sunflower, alfalfa, potato, tomato, tobacco, grape, barley, pea, bean, field bean, lettuce, cotton, sugar cane, sugar beet, broccoli or other vegetable brassicas or poplar.

The invention also extends to harvestable parts of a plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins. The invention also relates to food products and food supplements comprising the plant of the invention or parts thereof.

To the extent that any experimental or practical techniques needed are not provided in the specific examples, they are available as a matter of common general knowledge in the art to a person of ordinary skill. For example, with reference to (1) J. Sambrook, E. F. Fritsch, and T. Maniatis, 1989, “Molecular Cloning: A Laboratory Manual” Second Edition, Books 1-3, Cold Spring Harbor Laboratory Press; (2) J. M. Polak and James O'D. McGee, 1990, “In Situ Hybridization: Principles and Practice” Oxford University Press; (3) B. Roe, J. Crabtree, and A. Kahn, 1996, “DNA Isolation and Sequencing: Essential Techniques”, John Wiley & Sons; (4) Ausubel, F. M. et al. (1995 and periodic supplements “Current Protocols in Molecular Biology” chapters 9, 13, and 16, John Wiley & Sons, New York, N.Y.); (5) J. Lilley and J. E. Dahlberg, 1992, Methods of Enzymology: “DNA Structure Part A: Synthesis and Physical Analysis of DNA Methods in Enzymology” Academic Press; (6) M. J. Gait (Editor), 1984, “Oligonucleotide Synthesis: A Practical Approach” IRL Press; and (7) E. M. Shevach and W. Strober, 1992 and periodic supplements, “Current Protocols in Immunology”, John Wiley & Sons, New York, N.Y. Each of these general texts is herein incorporated by reference.

### Example 1: Generation of IQD22 (At4q23060) Overexpression (OE) Lines in Arabidopsis thaliana Col-0

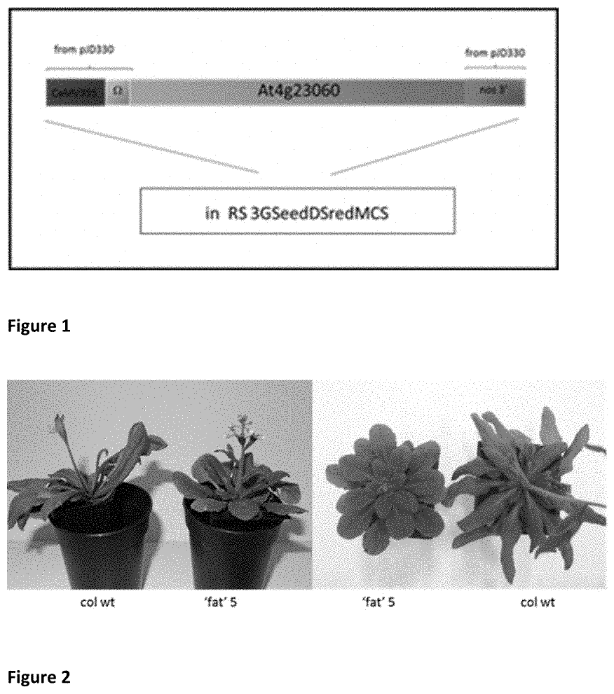

Arabidopsis lines were generated which overexpress IQD22 under the constitutive CaMVS35 promoter. FIG. 1 shows a schematic diagram of the genetic construct used to transform Arabidopsis thaliana Col-0.

The preparation and construction of the genetic constructs and the transformation and culturing up and reproduction of transgenic plants was as follows:

The clone containing the IQD22 cDNA was ordered from NASC. After confirmation of the full length cDNA sequence, the cDNA was amplified with Primer NcoI F: ccatggGAAAAG CGTCACGGTG GTT [SEQ ID NO: 57]; and Primer EcoRV R: gatatc tcagtacctatacccaattggcat [SEQ ID NO: 58]; and subcloned into the TOPO vector (Invitrogen). The fragment containing the IQD22 fragment was excised using the restriction enzymes NcoI and EcoRV and cloned into the vector pJD330 which had been digested with NcoI/SmaI, thereby inserting it between the S35 promoter and the nos terminator. The fragment containing S35:IQD22:nos was excised from pJD330 using BamHI/BglII and cloned into the binary transformation vector BIN19. Transformation of Arabidopsis thaliana col-0 was performed using the floral dip method (Clough S J and Bent A F, Plant J. 1998 December; 16, 735-43). Plants containing the transgene were selected on kanamycin plates (generation T1) and taken forward to homozygosity (T3). Experiments were performed with homozygous T3 plants.

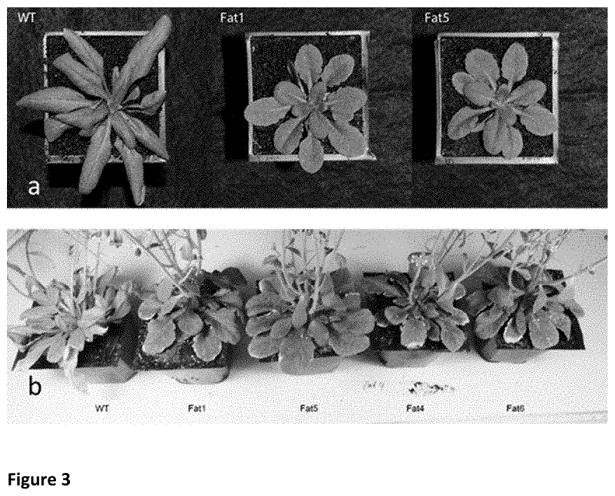

Out of twelve positive transgenic plants, four plants (30%) showed a noticeable phenotype with rounder, thicker and darker green leaves (see FIGS. 2 and 3a). Once flowering, also the inflorescence stem showed a remarkable increase in thickness (see FIGS. 2 and 3b).

FIG. 2 shows 4-5 weeks old Arabidopsis plant constitutively overexpressing IQD22. The altered phenotype line Fat 5 is shown here in comparison with the col-0 WT.

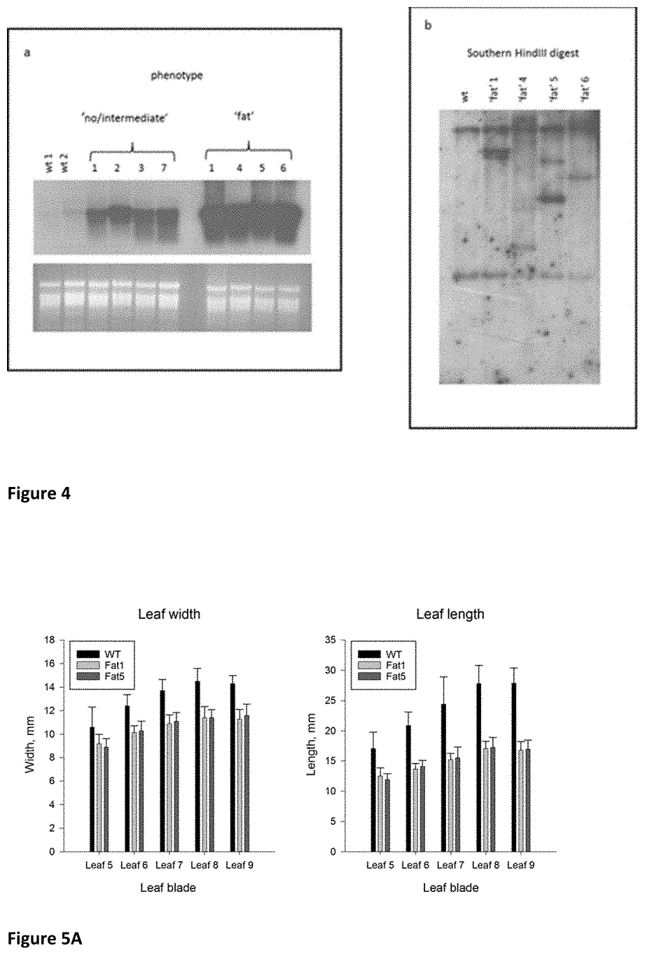

This alteration in growth is directly correlated with the expression level of the transgene as confirmed by Northern blotting as shown in FIG. 4a, with all the lines showing the ‘Fat’ phenotype having a very high expression level. Southern blots as shown in FIG. 4b ruled out that these high expression levels were due to a positional effect of one particular insertion event of the transgene into the Arabidopsis genome, as it was confirmed that ‘Fat’ lines 1, 4, 5 and 6 were each the result of independent insertion events. ‘Fat’ phenotype of At4g23060 OE is therefore linked to expression level of the transgene (FIG. 4a) and is the result of independent insertion events of the transgene (FIG. 4b).

### Example 2: Characterisation of the Effects of Constitutive IQD22 OE in Arabidopsis on Whole Plant Morphology

The photographs of FIG. 3 compare the rosette phenotype of WT and Fat1 and Fat5 OE Arabidopsis plants; (a) showing rosette size of WT and IQD22 OE lines after 33 days of growth, and (b) after 49 days of growth. Under growth conditions of 16 hrs light, 200 μmol sec⁻² mm⁻² the growth of the IQD22 OE lines was delayed when compared to WT at 33 days, but then reached similar size in due course after 49 days as shown in FIG. 3b.

Measurements of the overall inflorescence height of 49 days old plants showed that stem height in the OE lines was reduced to 53% compared to wild type (see table 1 below). There was no change in internode number but there was a large reduction in internode length.

The stem diameter measured 1 cm above the rosette was increased by 60% in the IQD22 OE lines compared to WT (see table 1 below). Resin embedded stem cross sections shown in FIG. 5 reveals how increase of stem diameter is the result of an increase in cell number rather than cell size.

TABLE 1
Morphology of inflorescence stems of wild type
and IQD22 overexpressing lines Fat1 and Fat5.

| Main inflorescence stem | Wild type | Fat1 | Fat5 |
|---|---|---|---|
| Height (cm) | 36.1 ± 3.4 | 19.2 ± 2.4 | 18.8 ± 2.2 |
| Cauline branch number | 4.9 ± 0.9 | 5.2 ± 0.7 | 5.2 ± 0.7 |
| Diameter (mm) | 1.68 ± 0.26 | 2.8 ± 0.4 | 2.7 ± 0.3 |
| First internode | 4.0 ± 1.6 | 2.5 ± 1.4 | 2.6 ± 1.2 |
| Second internode | 3.3 ± 0.7 | 1.4 ± 0.8 | 1.1 ± 0.5 |
| Third internode | 3.4 ± 0.9 | 1.5 ± 0.8 | 1.4 ± 0.7 |

Data are mean values ± standard deviation (WT n = 14, Fat1 n = 24, Fat5 n = 20).
Plants are 49 days old.

The rosette phenotype obtained when IQD22 is overexpressed in Arabidopsis has significantly altered leaf shape, not being as elongated as WT and rounder than WT. As can be seen in FIG. 5, the leaf blade width is reduced to about 80% of WT and the leaf length to about 60-70% compared to WT.

The IQD22 OE plant leaves are much thicker than WT. When they are bent they tend to snap and appear juicy—in a sense it is like the plant has become more of a succulent phenotype. Indeed, as shown in FIG. 5B and FIG. 14, a comparison of the water content of WT and OE lines shows that OE lines contain more water. After drying, leaves of OE lines have less dry matter mass.

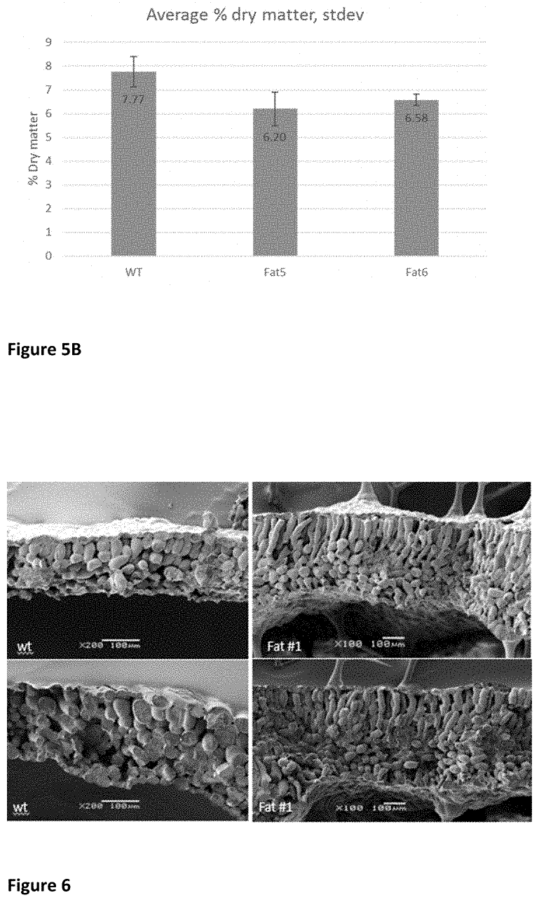

FIG. 6 shows a series of micrographs of freeze fracture SEM Arabidopsis leaves from WT and Fat1 overexpressing plants. The thicker character of the overexpressing plant leaves is clearly seen.

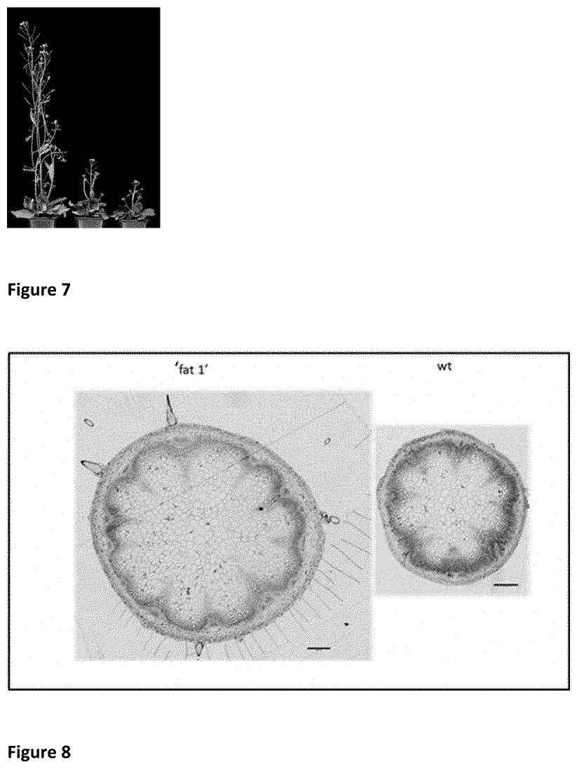

FIG. 7 is a photograph comparing WT and OE plants after 49 days growth. The OE plants can be seen clearly to be less than half the height of the WT.

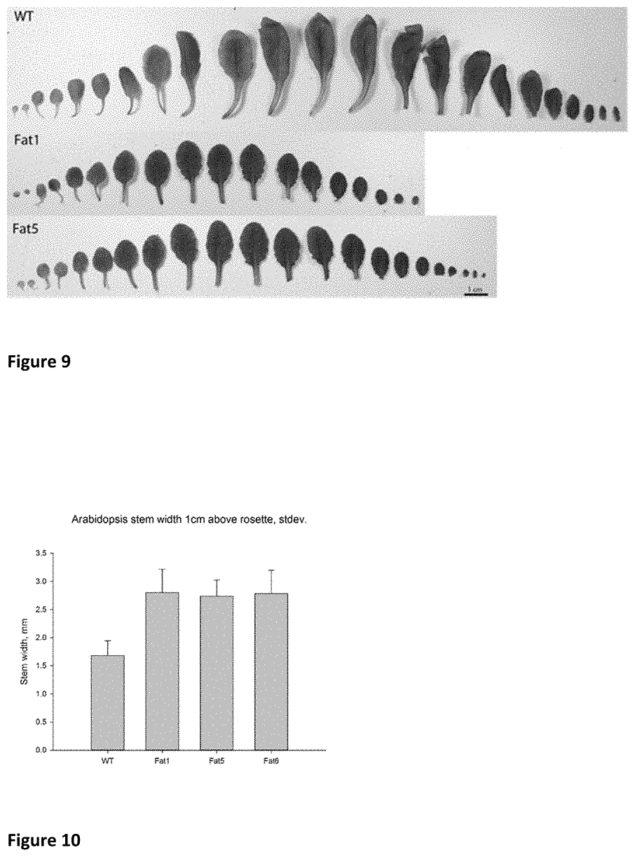

FIG. 8 shows stained sections of IQD22 OE line Fat1 and WT. The remarkable difference in size appears due to increased cell number, not increased cell size. Also, in the ‘Fat’ lines there are more vascular bundles.

FIG. 9 is a photograph of all the leaves of WT compared to Fat1 and Fat5 OE plants and provides a clearer overview of the entirety of the leaves for each plant.

FIG. 10 is a chart of comparing measured stem widths from WT and Fat 1 and Fat5 plants, wherein the stem is measured 1 cm about the rosette.

The IQD22 OE lines senesce later than the WT (not measured, observed visually). Leaf shape of the IQD22 OE lines is altered. The length of the leaf blades of OE is reduced to 70-60% of WT and petioles showed a length reduction of 15-33% compared to WT. Leaf width in the OE lines is also reduced to 80% of WT at 33 days.

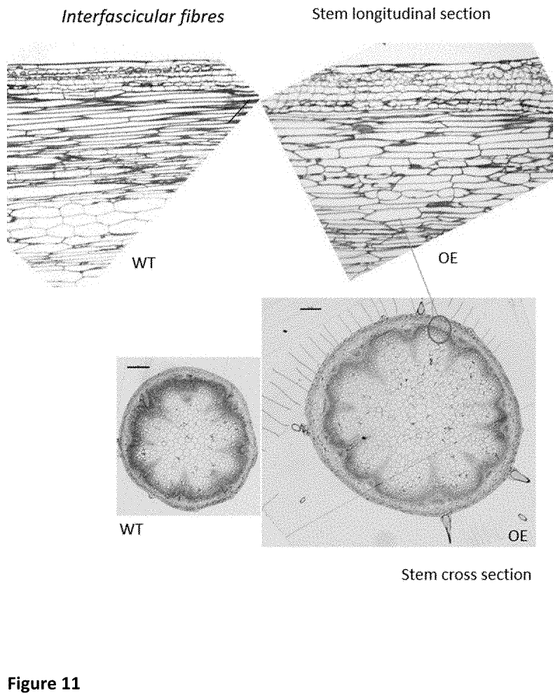

The height of the OE plants are 50% of WT. However, when considering the cells in the stem and in particular the interfascicular fibres (between the vein areas), these are shortened, as can be observed in FIG. 11.

The effects on the stem of OE plants is interesting. The stem is generally shorter and shows significant increase in cross sectional diameter being 60% bigger, but this is due to an increase in the number of cells rather than size of cells.

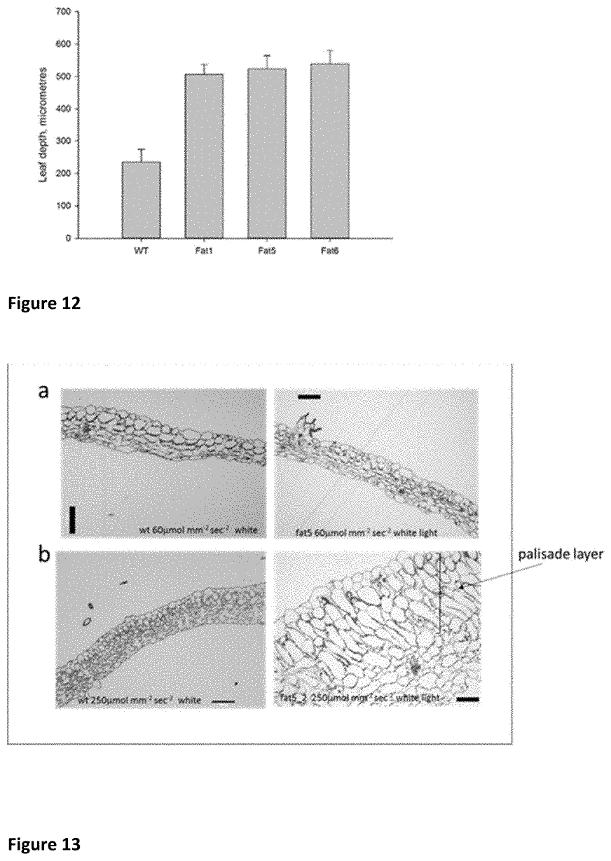

FIG. 12 shows how leaf thickness is increased in the IQD22 OE lines compared to WT. Leaf depth measured on transverse cross sections across the midpoint of the leaf lamina avoiding the midrib. Plants were grown under 16 hours daylight and 250 μmol mm⁻² sec⁻² of white light and analysed with the metaphor software (n=6). The thickness measured at the midpoint of the leaf lamina is increased by 2.23 times in the IQD22 OE lines when compared to WT.

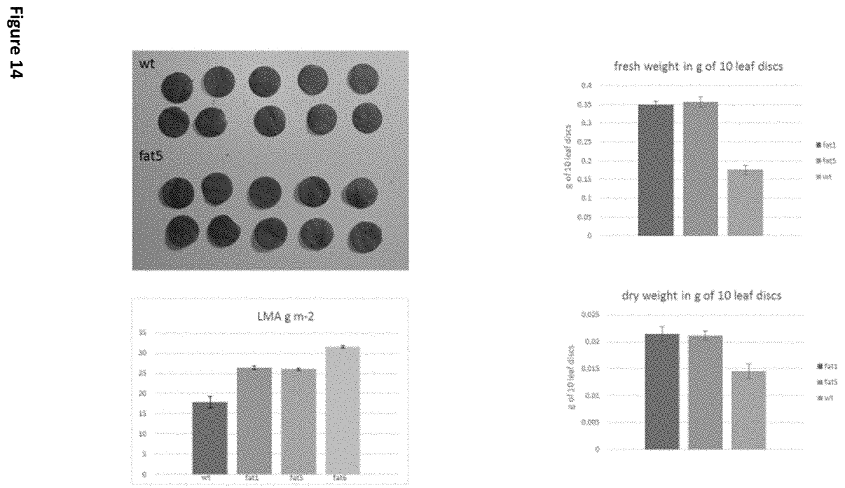

For overexpressing plants where IQD22 is under the control of native promoter, and when grown under low white light conditions (60 μmol mm-2 sec-2) FIG. 13 (a) shows no difference in leaf thickness was observed between the WT and the IQD22 OE lines. FIG. 13 (b) shows that under higher light intensity (250 μmol mm⁻² sec⁻²) the palisade tissue layer was composed of more cell layers and the cells within this tissue were much more elongated in the IQD22 OE lines when compared to WT. So, interestingly, the increased thickness of the leaf in the transgenic lines is therefore a light controlled trait. In addition, when grown under 250 μmol mm⁻² sec⁻² of white light, the palisade tissue in the IQD22 OE lines was 3-4 cell layers deep whereas in the WT this layer consisted of only 1 row of cells. Furthermore, the cells making up the palisade tissue in the IQD22 OE lines are extremely elongated. Both these factors are the main contributors to the observed increased thickness of the leaves.

The increased leaf thickness between wt and IQD22 overexpressing lines is reflected in an increase of leaf mass per unit area in the latter (see FIG. 14). Leaf mass per area (LMA) is calculated as the leaf dry mass per unit leaf area. It is an important leaf trait widely used in plant ecology and other disciplines for linking light capture to plant growth and carbon gain. To determine LMA, 5 plants per line were chosen, from each plant two leaves were taken and from each leaf two discs of identical diameter were punched out. Doing this, 10 leaf discs (e.g. as shown in FIG. 14) were obtained for each sample allowing the determination of surface area and the dry weight of these discs was measured. As shown in FIG. 14, the LMA for IQD22 OE line fat1 and fat5 was 35% higher than the LMA for WT. The highest LMA was measured for line fat6 (ca 47% higher than WT LMA), however this high LMA value is offset by the reduced size of the rosette of this line.

### Example 3: IQD22 is Expressed in the Palisades in Arabidopsis

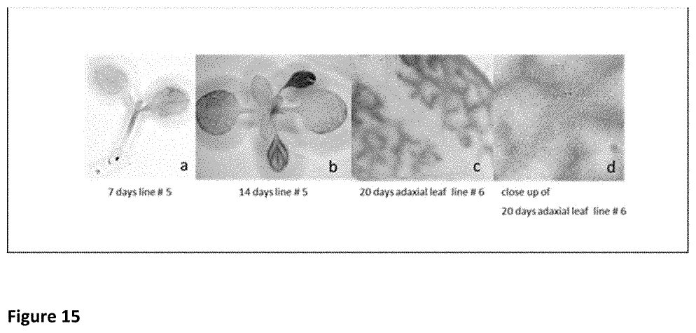

In order to investigate where IQD22 is expressed in the plant, 1.6 kb of 5′ upstream sequence of the IQD22 gene, comprising the promoter sequence, were amplified and fused to the reporter gene GUS. With this construct, stable transgenic Arabidopsis lines were generated. In 7 day old seedlings GUS expression was detected in the hypocotyl and in what at first impression looked like vasculature (FIG. 15a). However, as the plants grew older, it became evident that GUS staining indeed followed the vasculature but higher magnification revealed that GUS staining was localised to cells above the vessels. Shape and arrangement of those cells are characteristic for cells of the palisade tissue (see FIG. 15d). The significance of this pattern of IQD22 expression is currently not known. No staining was detected in the roots.

### Example 4: IQD22 Expression is Localised to Microtubules within the Palisade Cells in Arabidopsis

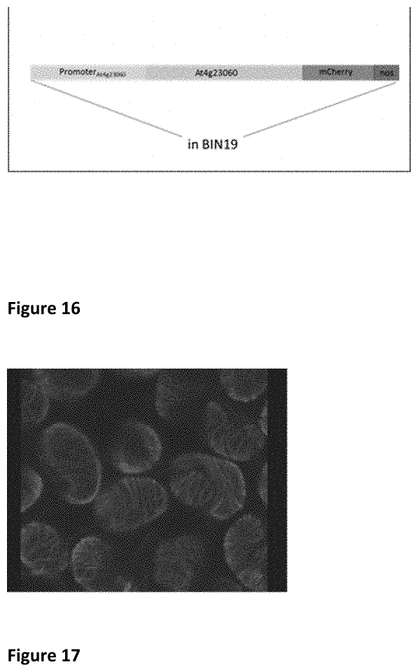

The same IQD22 promoter fragment used for the GUS reporter construct was cloned in front of IQD22 which had been translationally fused at its 3′ end to the fluorescent protein mCherry (see FIG. 16).

With this construct stable transgenic Arabidopsis lines were generated and analysed. The fluorescent fusion protein was associated with the cytoskeleton of the palisade cells (FIG. 17) with the microtubule network as has been demonstrated for proteins of other members of the IQD family.

### Example 5: Expression of IQD22 Under its Own Promoter Alters Leaf Shape and Results in Flattened Leaf Laminae Cells in Arabidopsis

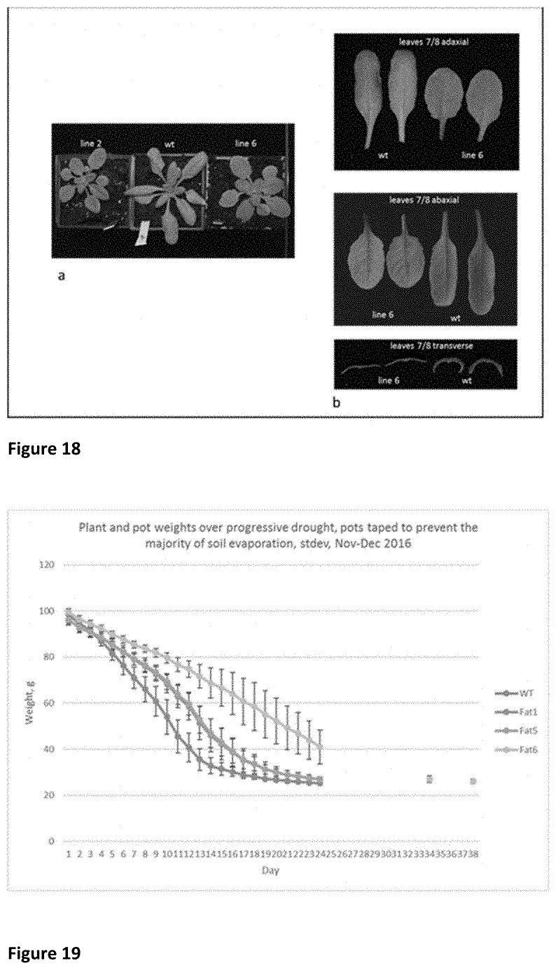

The transgenic plants which were used for the localisation studies described in the previous section also showed an altered phenotype. Compared to the elongated leaf shape of the wildtype the leaves were much rounder and also felt thicker to the touch. In addition, the typical epinastic curling of the leaves of the wildtype is completely abolished in the lines expressing IQD22 under the control of its own native promoter (see FIG. 18). This could potentially be a trait of interest as the surface area exposed to photosynthetically active radiation (PAR) is enlarged. Stems thickness however was not increased.

### Example 6: Response of WT and IQD22 OE Arabidopsis Lines to Drought

It is known that plants adapted to growth in arid habitats frequently are characterised by thicker leaves. As high IQD22 OE Arabidopsis lines had much thicker leaves and stems than WT, it was investigated whether the OE lines also had an advantage over the WT to cope with water stress. Two independent transgenic lines were chosen (‘fat1’ and ‘fat5’). Pots were filled with equal amounts of compost and plants grown under a normal watering regime for 4 weeks. Before starting to withhold water, pots were weighed again and brought to an equal weight by adding water. In addition, sellotape was placed over the compost in order to minimise water loss through evaporation from the compost surface. During the period of the progressive drying of the pots, pots were weighed daily and pictures were taken to record the response of the different lines. The experiment was repeated twice.

Recording the daily weight loss of the pots, the first observation made was that water loss of the pots containing the IQD22 OE lines was much reduced when compared to WT as they consistently weighed in heavier than the pots containing the WT plants (see FIG. 19). This could mean that the transpirational pull of water from the root through the plant is reduced in the IQD22 OE plants.

FIG. 19 shows how the OE lines show a slower rate of water loss over progressive drought. The rate of water loss over days 1-9 is as follows: WT=4.79 ml/day, Fat1=2.91 mL/day, Fat5=2.83 mL/day. From day 10-17 the rate is: WT=3.39 mL/day, Fat1=4.85 mL/day, Fat5=4.95 mL/day. (Fat6 appears slightly different because it is a much smaller plant than the other lines.)

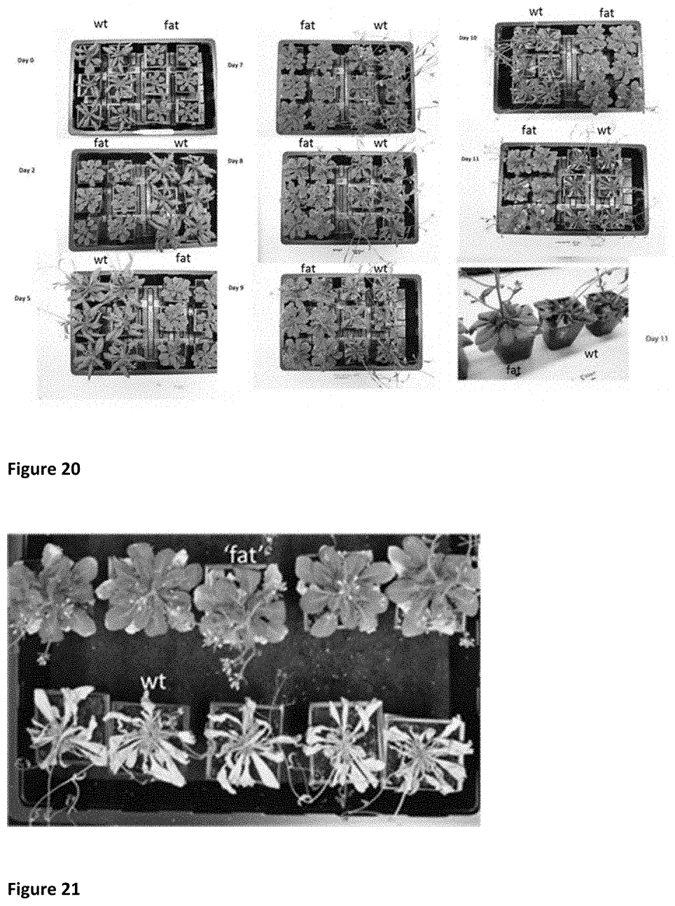

Secondly, as the series of pictures in FIG. 20 shows, progression of wilting after withholding water is much delayed in the IQD22 OE lines. By day 11, when the experiment was stopped, the WT plants were shriveled and dried up whereas the IQD22 OE lines were still green although the leaves started to lose some of their turgor.

After 11 days of no watering, pots were thoroughly saturated with water and a normal watering regime was resumed for a couple of days. As can be seen in FIG. 21, IQD22 OE lines had fully recovered after two days of re-watering, whereas the WT had not been able to do so.

### Example 7: Gene Expression in Arabidopsis WT and IQD22 OE Plants

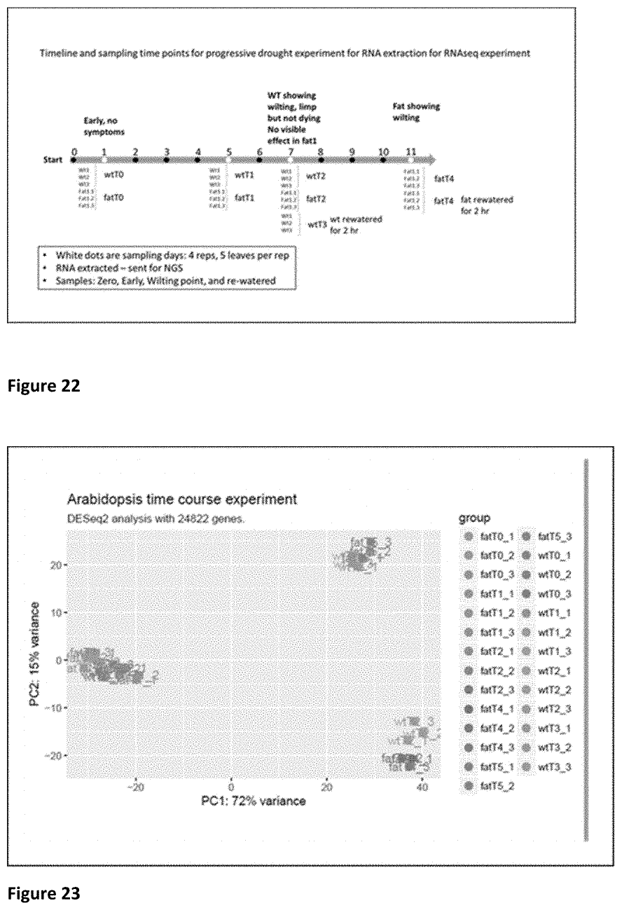

The varying ability of the WT and IQD22 OE plants to cope with progressive drought was investigated by looking at gene expression. RNAseq was performed with RNA obtained from leaf samples at different time points during the imposition of the stress. FIG. 22 details the setup of the experiment and indicates at which time points samples were taken for RNA extraction.

As can be seen in FIG. 22, leaf samples for RNA extraction were taken at: day 1 after withholding water: (plants and pots still saturated), WT and OE were sampled (wtT0 and fatT0); day 5 after withholding water: WT and OE were still showing no obvious signs of wilting (wtT1 and fatT1), day 7 after withholding water: WT showed signs of wilting (wtT2) whereas OE line maintained its turgor (fatT2). At that point WT was re-watered and samples taken after 2 hours (wtT3). Day 11 after withholding water for OE line only: point of wilting for OE lines (fatT4) and 2 hr after re-watering of the OE lines (fatT5). For a sample, an equivalent leaf was taken from five individual plants and pooled to create 1 replicate. 3 replicates were sampled. RNA was extracted and sequenced.

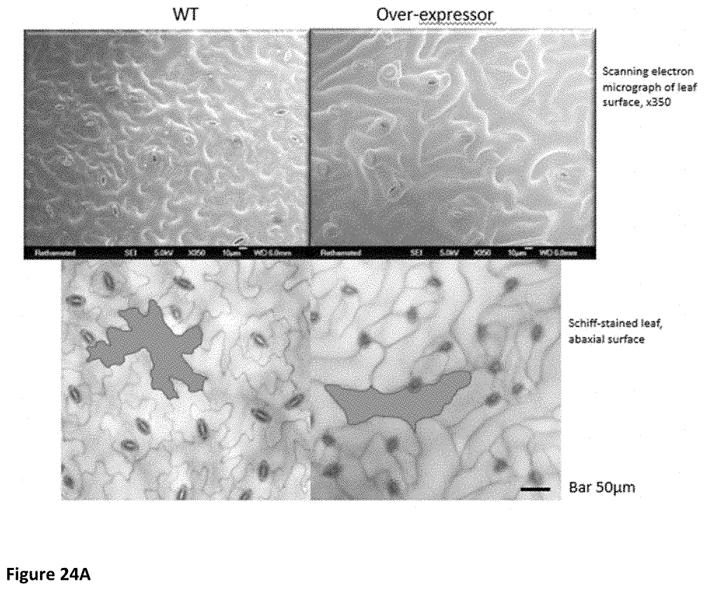

As the PCA plot in FIG. 23 shows, the visually observed different behaviour pattern of WT and OE to progressive drought is reflected in the RNAseq data. For example, whereas WT samples taken at timepoint T2 (showing signs of wilting) form a very distinct cluster, the IQD22 OE line sampled at the same timepoint, fatT2, still clusters with the fully watered samples corroborating the visual observation that the plants are unaffected by the water stress at this stage. Once the wilting point for the transgenic line is reached (fatT4), the cluster generated at this sampling time point forms its own discrete cluster in the vicinity of the wtT2 cluster. The formation of two discrete clusters for WT and transgenic line at the point when wilting of the leaves sets in points towards underlying differences in gene expression.

### Example 8: Measurement of Abaxial Leaf Cell Characteristics on WT and IQD22 OE Arabidopsis Plants

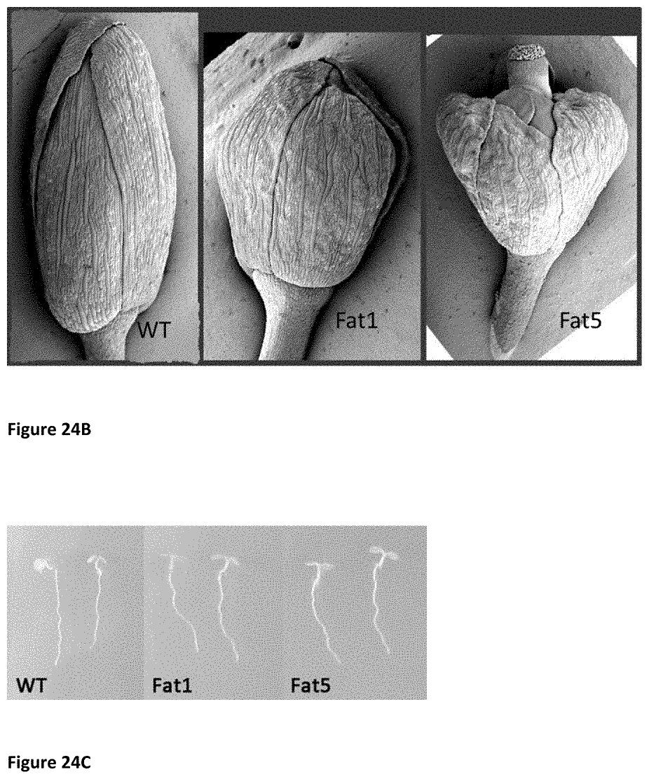

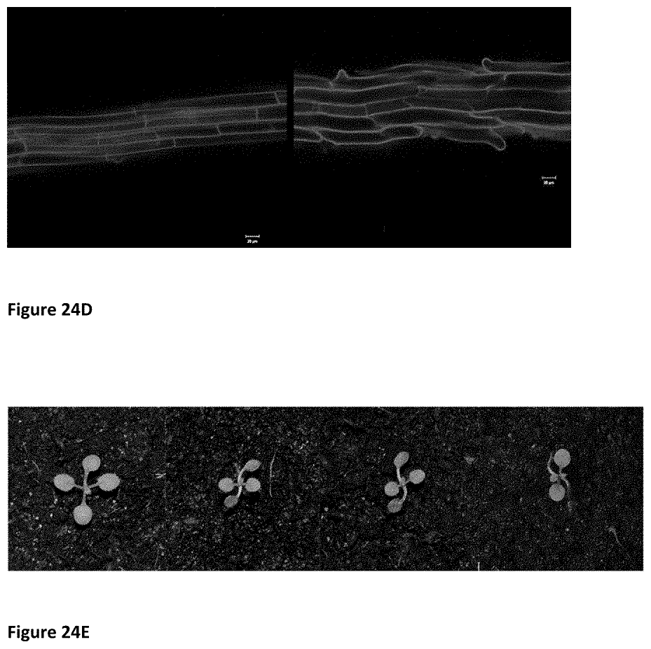

Looking at FIG. 24A, the adaxial leaf surface cell morphology is quite drastically altered in IQD22 OE line, compared to WT. The OE lines have greatly reduced lobing of the epidermal cells instead of the classic jigsaw shape. Although only the abaxial surface is show in FIG. 24A, the same change in cell shape is also observed between WT and OE in the adaxial leaf surface as well. The same cell shape change between WT and OE is also seen on the petals.

### Example 9: Other Miscellaneous Morphological and Cellular Observations as Between WT and IQD22 OE Arabidopsis Plants

FIG. 24B shows how the floral organs are shortened in IQD22 OE lines compared to WT plants. In the OE plants, the abaxial petal surface has “jigsaw” type cells but show no lobing effect like in the leaf epidermal cells.

FIG. 24C shows Arabidopsis seedlings grown on vertical agar plates. The roots of the Fat1 and Fat5 OE lines show a right-hand bias, whereas the roots of WT normally shows a slight left hand bias. Closer inspection of the roots (see FIG. 24D) using propidium iodide staining to highlight cell walls for microscopy, shows that there are cell shape defects, with the ends curling out of the plane of the surface, cells appear shorter, dividing walls are more angled, roots are also thicker.

FIG. 24E shows how cotyledon petioles and first leaves in Fat1, Fat5 and fat6 OE lines are twisted compared to WT. Later leaves in the OE lines are not twisted.

### Example 10: Measurement of Stomatal Conductance on WT and IQD22 OE Arabidopsis Plants

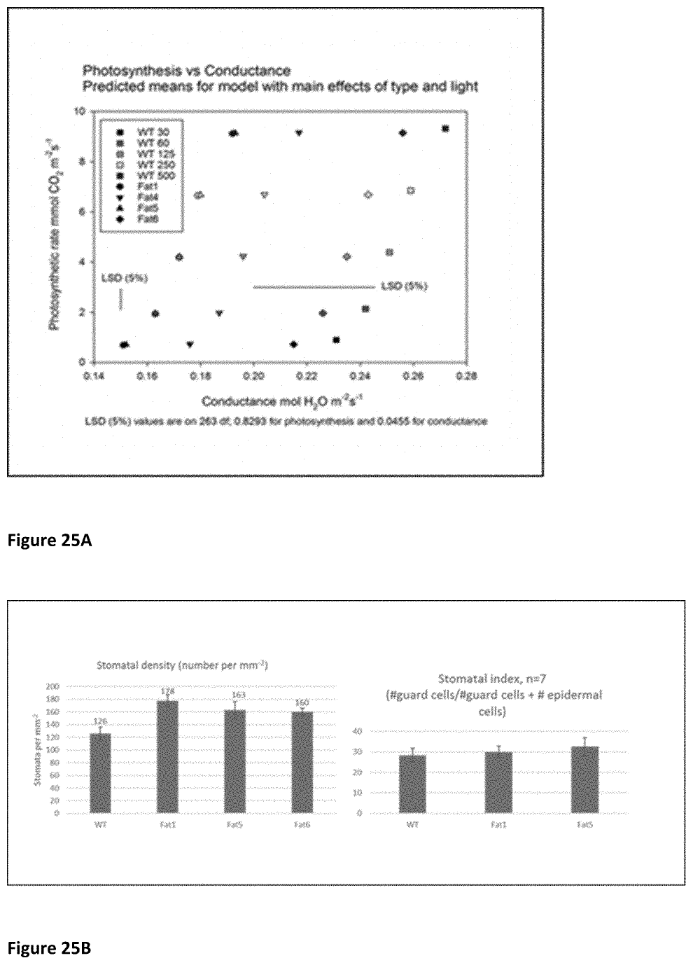

To test whether differences in gas exchange between the WT and IQD22 OE lines could be detected measurements with the LI-6400XT Portable Photosynthesis System were performed. Data was collected at PAR (photosynthetic active radiation) levels of 500, 250, 125, 60 and 30 (μmol (photons) m⁻² s⁻¹) with 500 μmol m⁻² s⁻¹ representing saturating levels of PAR. Conductance and photosynthesis rate was measured at each light level and plotted against each other. The method of residual maximum likelihood (REML) was used to fit a multivariate linear mixed model to the photosynthesis rate and conductance data combined, testing (F-tests) for the main effects and interactions between the factors of light level and genotype, and accounting for the structure of the variance-covariance matrix underlying the data from the two variables. The plots thus obtained for IQD22 OE lines ‘fat1’ ‘fat5’ and ‘fat4’ are significantly different to WT with regard to conductance and show at the same photosynthetic rate a reduced conductance (see FIG. 25A). Measurements for line ‘fat 6’ put it closer to WT with a slight shift to lower conductance values, however line ‘fat 6’ is a unusual it its growth habit to the other fat lines. This reduced stomatal conductance could explain the reduced water loss observed during the drought experiment.

### Example 11: Measurement of Stomatal Numbers in WT and IQD22 OE Arabidopsis Plants

In order to investigate whether a reduced number of stomata could explain the observed decrease in water loss of the IQD22 overexpressing lines, stomata numbers per mm⁻² and stomatal index were determined. Although a higher number of stomata were counted per mm⁻² leaf area for the IQD22 overexpressing lines, when stomatal index was measured no differences for those lines compared to wt were observed (FIG. 25B). Stomatal density (SD) is a function of both the number of stomata plus the size of the epidermal cells. Thus, SD is affected both by the initiation of stomata and the expansion of epidermal cells. This expansion is a function of many variables (e.g. light, temperature, water status, position of leaf on crown and intra-leaf position) and makes direct comparison between different samples difficult. In order to normalise for the effects of this expansion (i.e. density of epidermal cells) the concept of stomatal index is used. Reduced water loss in the IQD22 OE lines is not due to a decreased density of stomata.

### Example 12: Generation of IQD23 and IQD24 OE Arabidopsis Lines

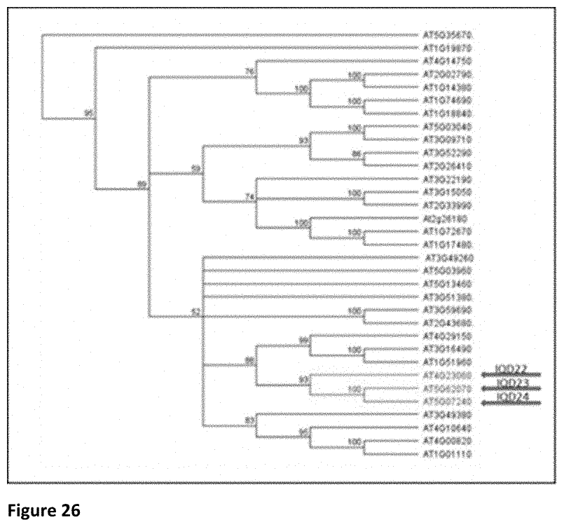

In Arabidopsis thaliana the two closest homologues of IQD22 are IQD23 (At5g62070) and IQD24 (At5g07240) (FIG. 26). As it was of interest whether these two homologues conferred similar characteristics to Arabidopsis as IQD22, overexpressing lines for those genes were generated. Expression of IQD23 and IQD24 was controlled by the S35 CaMV promoter.

Both IQD23 and IQD24 were cloned and tested whether they confer similar traits to Arabidopsis as IQD22 when overexpressed.

As is the case for IQD22, transgenic plants with thicker leaves were obtained for the S35:IQD23 and S35:IQD24 OE constructs. In both cases, not all the transformed plants displayed the phenotype of increased leaf thickness. This trait was shown to be related to expression levels of the transgenes. IQD23 and IQD24 OE also resulted in increases in leaf thickness and gave drought resistance compared to WT plants.

### Example 13: Response of WT and IQD23 OE Arabidopsis Lines to Drought

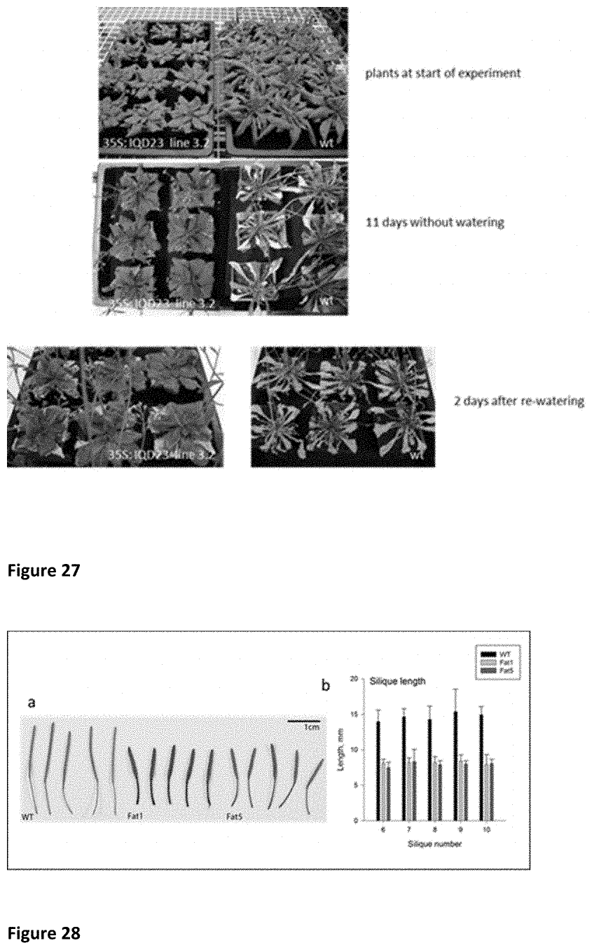

The drought experiment was set up as already described in Example 6 for IQD22 OE lines. After 4 weeks of growth under well-watered conditions in a long day cabinet (16 hours of daylight) watering was stopped and the response of the plants to progressive drought recorded. FIG. 27 shows that after 11 days of no watering plants of the IQD23 OE line were still green, whereas the WT showed severe signs of wilting. Subsequently, pots were drenched in water and the ability to recover from the stress was observed. For WT plants the imposed water stress had been too severe for them to recover. IQD23 OE lines however were able to recuperate. The ability to cope with water stress is increased in IQD23 OE lines.

### Example 14: Response of WT and IQD24 OE Arabidopsis Lines to Drought

Transgenic IQD24 OE were generated as described. As the case for IQD22 and IQD23, the IQD24 OE plants show increased leaf and stem thickness which is dependent on the expression level of the IQD24 transgene. The percentage of plants with a thicker phenotype obtained after the transformation was however lower than that for IQD23 and IQD22. The increase in thickness of leaves and stem is the trait that confers the ability to cope with water stress and so it is fully expected that the IQD24 OE lines also to do better under water scarce conditions compared to WT plants.

### Example 15: Effect of IQD22 OE on Seed Yield Compared to WT in Arabidopsis

In order to establish whether IQD22 overexpression has an effect on seed yield, 12 plants each for wt and IQD22 OE lines ‘fat1’, ‘fat4’ and ‘fat5’ were grown to maturity and traits affecting seed yield recorded. Measurements of the length of the siliques revealed that in the IQD22 overexpressing lines silique length was in average ca 40% shorter than in wt (FIG. 28).

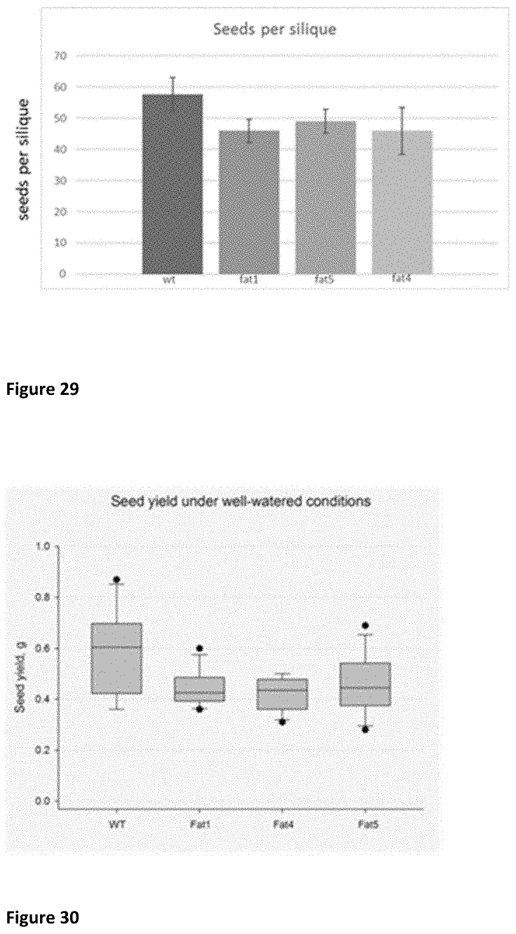

As a consequence of the reduced silique length, the number of seeds obtained per silique is also reduced in the IQD22 overexpressing lines (see FIG. 29). This reduction in seed number per silique however was around 20%-25%. Considering that the silique length reduction was ca 40% this would imply that the packing of seeds within the silique is tighter in the IQD22 OE lines.

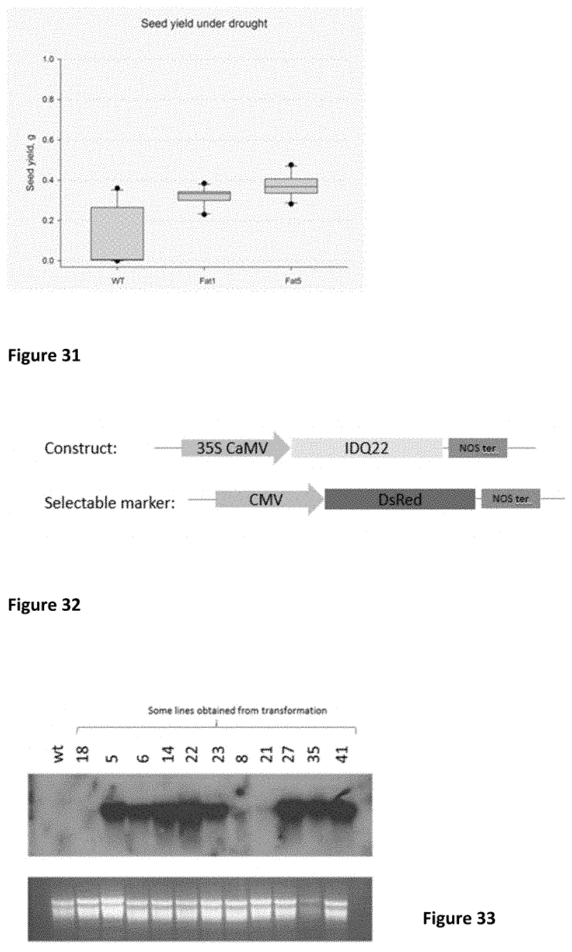

Total seed yield was reduced in the IQD22 overexpressing lines (see FIG. 30). This reduction was around 24%-29% compared to wt. This reduction is very likely a combination between a reduction of number of seeds per silique and number of siliques per plant, the latter however has not been determined.

Where the WT and IQD22 OE lines were subjected to a severe drought stress during their growing period however, the seed yield obtained from transgenic OE lines was much higher than the one obtained from WT (see FIG. 31). As described for the drought experiments, plants were grown for 4 weeks under well-watered conditions after which watering was stopped completely for 11 days. Subsequently, watering was resumed and plants grown to maturity and seeds harvested.

### Example 16: Generation of IQD22 (At4g23060) Overexpression (OE) Lines in Camelina Sativa ‘Celine’

The same construct as used in Arabidopsis Example 1 was used in this example Camelina sativa ‘Celine’. This is shown schematically in FIG. 32. The IQD22 gene was expressed under the control of constitutive 35S CaMV promoter. Plants were transformed by Agrobacterium floral dip method. Transgenic plants were selected for by DSRed expression in seeds and confirmed by PCR. As was the case for Arabidopsis, Northern blot of RNA extracted from selected lines showed a large signal indicating high expression of the transgene which was related to the strength of the phenotype shown (see FIG. 33).

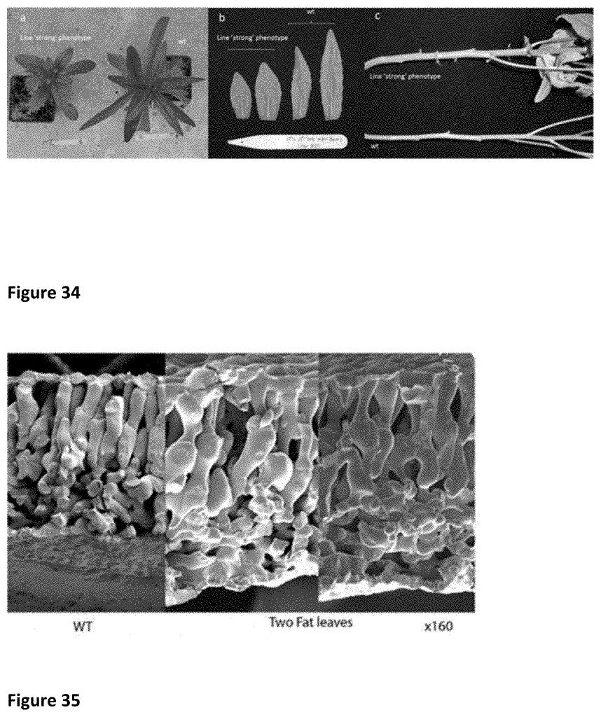

Transformants were grouped into ‘strong’ or ‘intermediate’ transgenic lines depending on the strength of the phenotype shown. Both are fertile. As shown in FIG. 34, lines assigned to the ‘strong’ phenotype group have rounded, thicker leaves, a stocky fat stem, the seed pod spacing is altered with the seed pods being rounder and plant maturing and seed ripening is delayed by about 2-3 weeks when compared to wildtype. Also, the OE plant is a shorter plant initially but then achieves similar height to WT nearer plant maturity.

The ‘intermediate’ type plants are between WT and the ‘strong’ OE phenotype, having longer, thinner (i.e. more lanceolate) leaves than the strong ones. The plants are of normal height but slightly later flowering than WT. The seed pod shape was the same as that observed for the ‘strong’ lines.

FIG. 35 shows Scanning electron microscopy (SEM) photomicrographs of freeze fractured IQD22 OE Camelina lines. The leaves of OE plants are thicker than WT. The size of the palisade cells in OE leaves is increased compared to WT.

### Example 17: Subjecting WT and IQD22 OE Camelina Lines to Drought Stress

This experiment was carried out in order to establish whether IQD22 OE confers the same enhanced ability to cope with water stress to Camelina as was observed for A. thaliana. Five plants displaying either the ‘strong’ or the ‘intermediate’ phenotype were planted out together with five wildtype plants. Before planting, it was ensured that all pots contained equal amounts of soil and had the same weight. After 4 weeks of growth, when flowers were about to open, the pot weights were brought again to equal weights with water and subsequently watering was stopped and the response of the plants to decreasing water availability was observed.

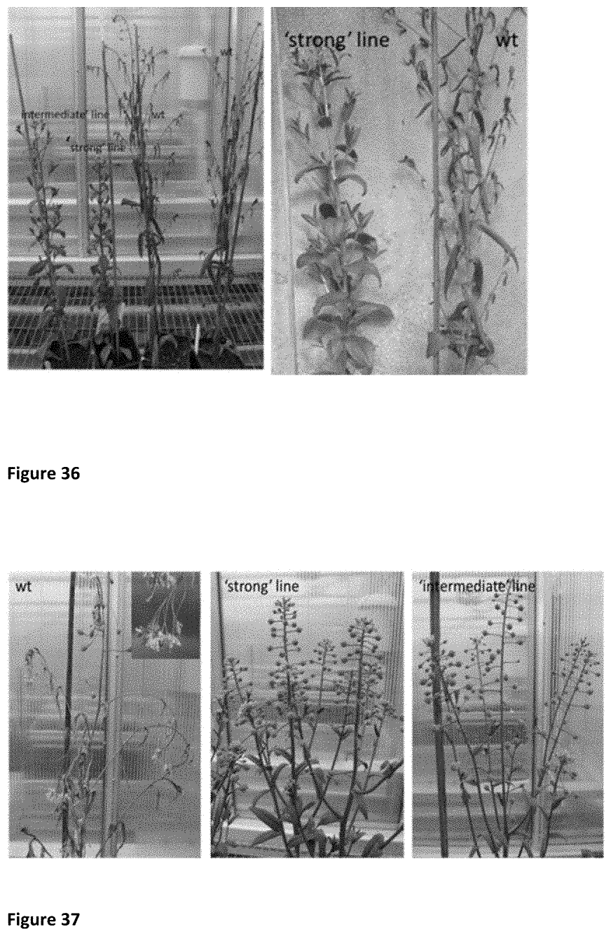

As reported for Arabidopsis, soil-drying occurred at a much slower speed in the case of the transgenic lines, suggesting a reduced transpiration rate for these plants. As is shown in FIG. 36, after 4 days of withholding water, WT Camelina showed already obvious signs of wilting whereas no effect on the transgenic plants was observed.

After 11 days without watering, WT Camelina showed distinctive stress symptoms such as severely wilted and yellowing leaves and drying of the inflorescences with shriveled flowers whereas the two transgenic lines still had green leaves (although they started to lose their turgor) and upright inflorescences. However, the ‘intermediate’ OE line showed stronger water-stress induced symptoms than the ‘strong’ line.

In both transgenic lines the water stress resulted in stunted inflorescences as the distance between seed pods was reduced when compared the growth habit of well-watered plants, as can be seen in FIG. 37.

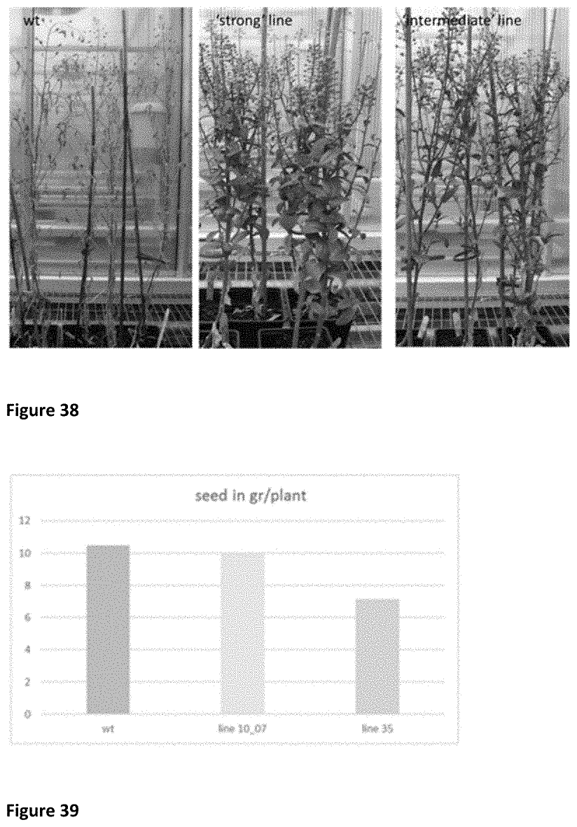

Watering of the plants was resumed after 11 days without water (after the pictures in FIG. 37 were taken). As is evident from FIG. 38, 11 days after re-watering the WT plants had been unable to recover from the water stress whereas both transgenic lines had almost fully recovered.

### Example 18: The Effect of IQD22 OE in Camelina on Seed Yield Compared to WT Plants

The results here are based on data obtained from 5 plants for wt, ‘intermediate’ and ‘strong’ phenotype plants respectively. Fully ripened seeds obtained from each plant were harvested and weighed. This was done for plants grown under optimal water conditions and also for the droughted plants described in Example 17.

As can be seen in FIG. 39, high levels of IQD22 expression (see line 35) results in a fall seed yield by about 30% compared to normal IQD22 expression levels in the wt. However, plants of the ‘intermediate’ phenotype (line 10-07), with lower levels of expression of the transgene, do not show much reduction in seed yield.

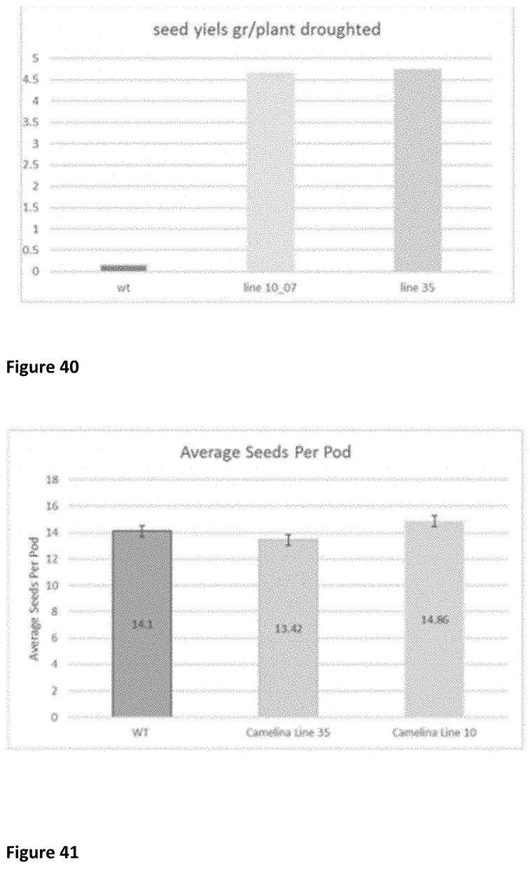

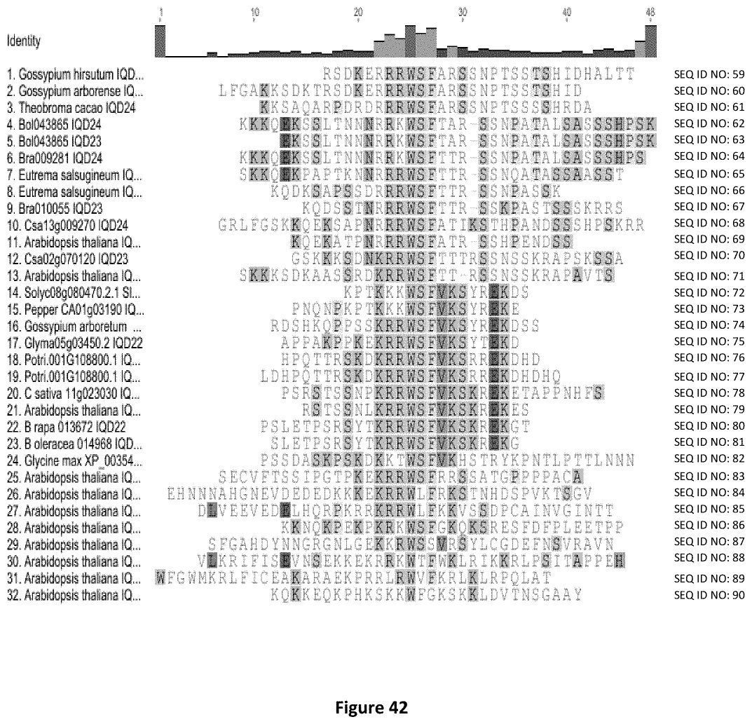

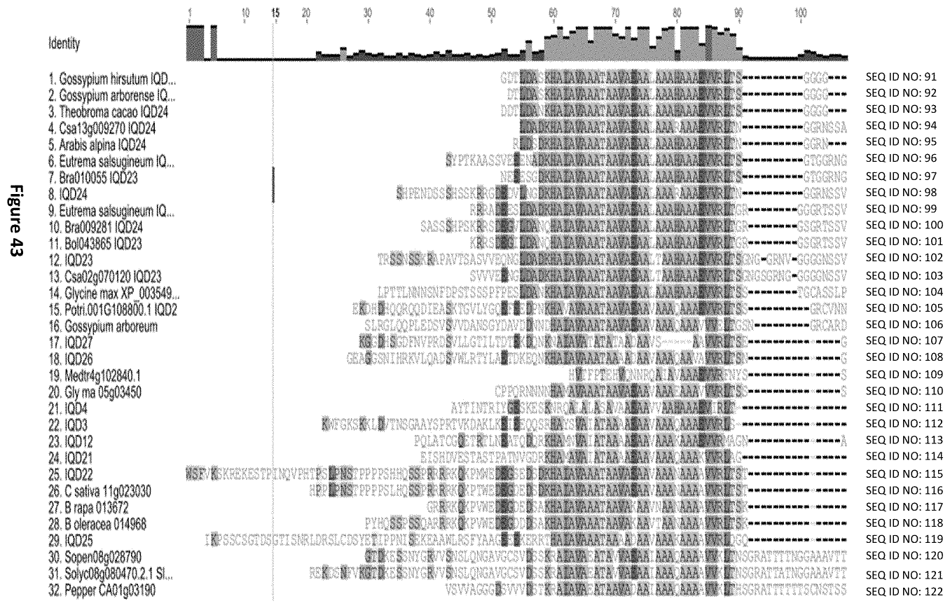

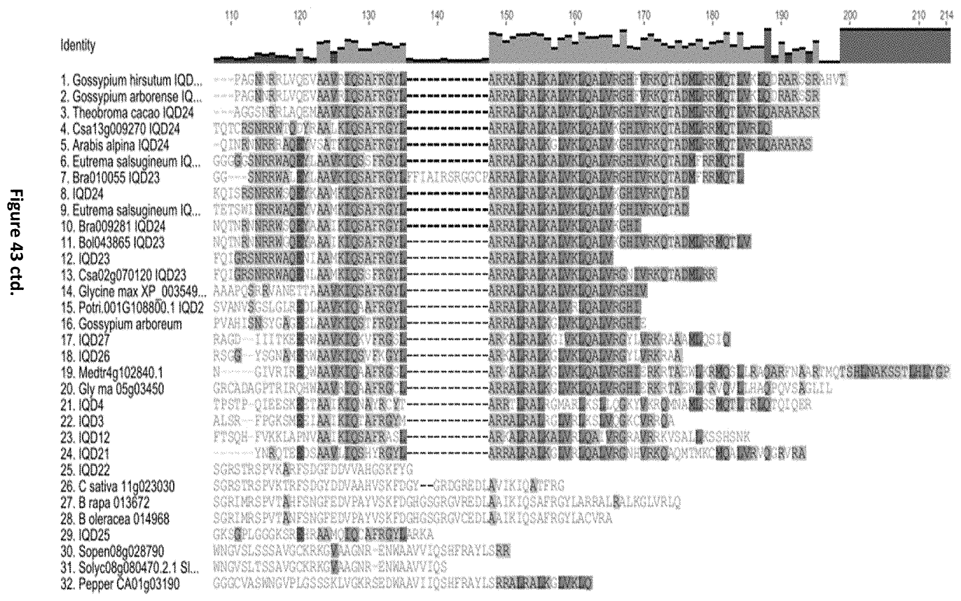

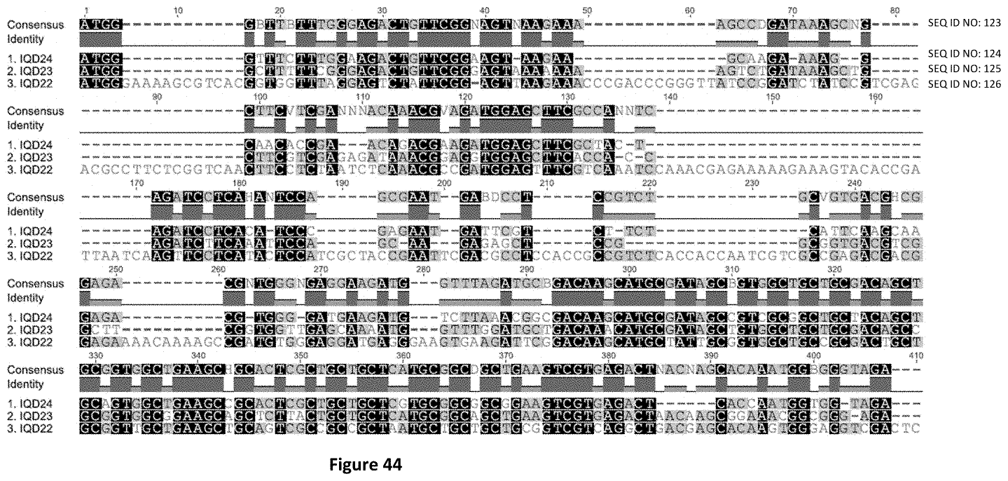

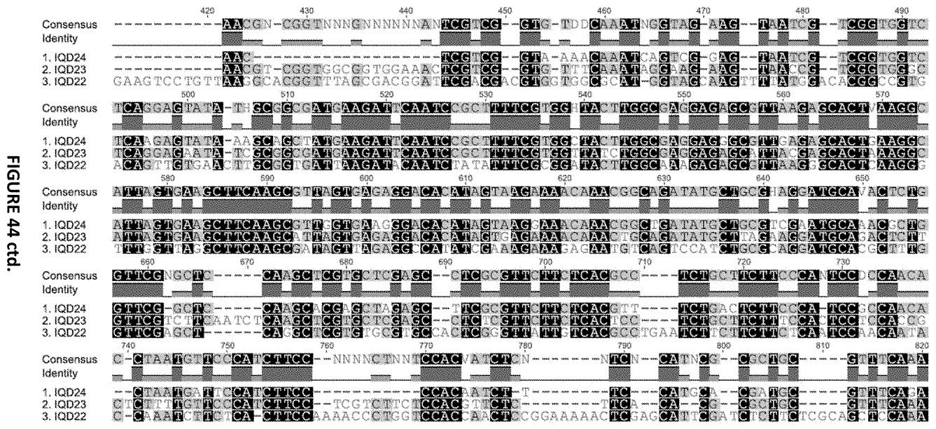

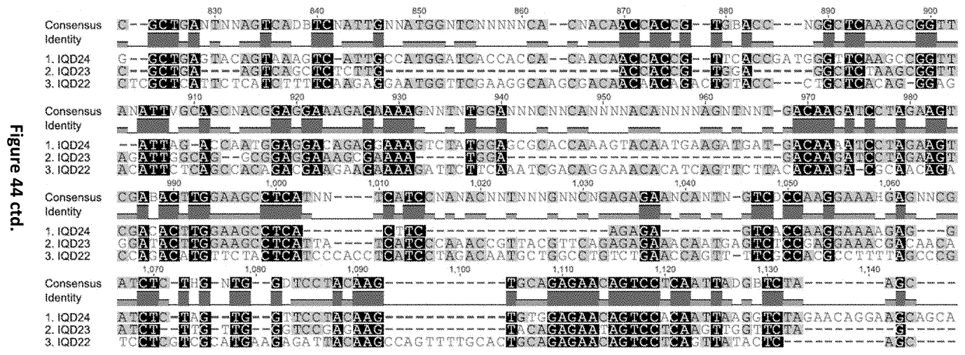

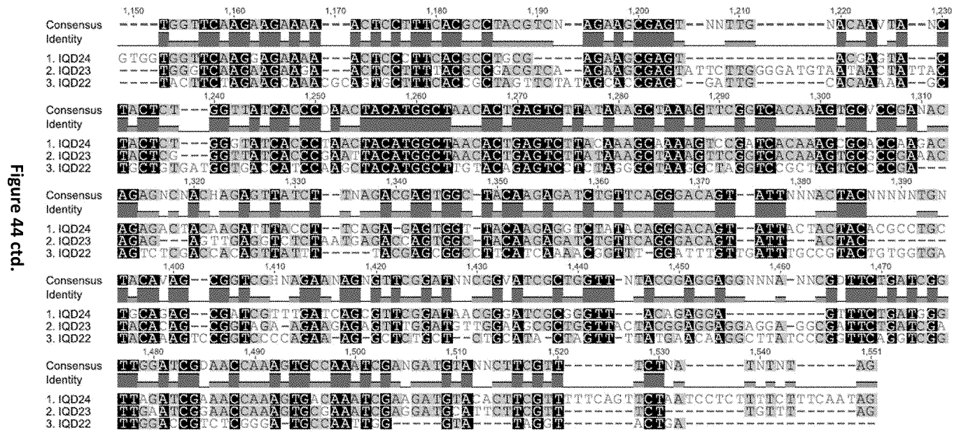

However, when plants were subjected to the severe water stress of withholding water for 11 days, after which normal watering was resumed, the IQD22-overexpressing lines performed much better than the wt plants (see FIG. 40). For wt plants the fall in seed yield was striking—almost 100%, whereas the transgenic plants still yielded about 50% their well-watered counterparts.

The reduction of seed yield observed in the well-watered transgenic lines compared to wt was not due to a reduction of seed number per seed pod (see FIG. 41) but possibly in the number of pods per plant.

### Example 19: The Native IQD22 Promoter Drives Palisade Cell Specific Expression in Leaves of Arabidopsis thaliana

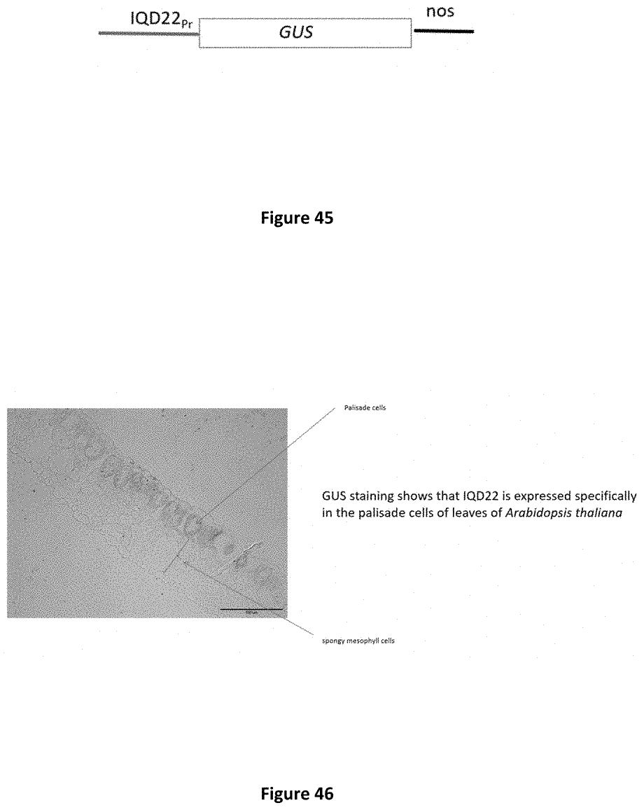

Transgenic A. thaliana plants are produced according to procedures described in Example 1, but the expression construct use comprises a native IQD22 promoter driving expression of a GUS reporter gene. The construct is shown in FIG. 45. Transgenic plants were grown and then leaves were harvested and sectioned. As shown in the micrograph in FIG. 46, the palisade cells stain specifically showing that the native IQD22 promoter is responsible for driving a palisade cell specific expression of the GUS gene. No GUS staining was observed in spongy mesophyll cells, for example.

### Example 20: IQD22 Overexpression in Poplar

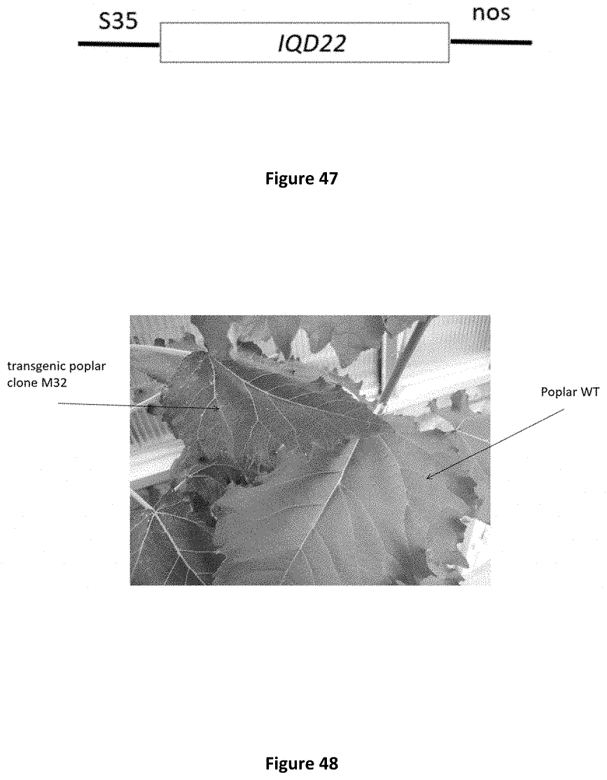

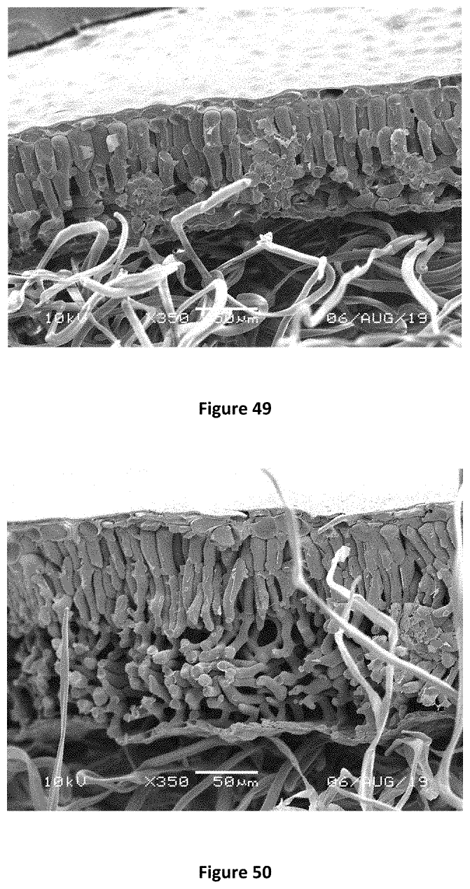

Wild type hybrid poplar (Populus tremula X Populus alba Institut National de la Recherche Agronomique (INRA) clone 7171-B) was used. Transgenic poplar plants were produced according to the procedures described in Example 1 using an expression construct comprising an S35 constitutive promoter, the Arabidopsis IQD22 gene and the nos termination sequence. The expression construct is shown in FIG. 47. WT and transgenic poplar plants were grown and compared at various stages of growth. When Arabidopsis IQD22 is expressed constitutively in poplar the leaves also become thicker. FIG. 48 shows transgenic clone M32 and WT leaves together and the transgenic leaf is darker green compared to the WT leaf. Scanning electron microscopy (SEM) confirms that leaf thickness is increased in the transgenic plant compared to WT. FIG. 49 shows freeze fracture of a WT leaf. FIG. 50 shows freeze fracture of leaf 14 of poplar transgenic clone M32A.

What the inventors have found with the transgenic plants made so far, is that when IQD22 is expressed constitutively (e.g. with a S35 promoter) the particular phenotype is related to the relative level of overexpression of the transgene. In practice, this means that some transgenic lines with high levels of expression show what is described herein as the ‘fat’ phenotype. However, when IQD22 is overexpressed using a native promoter, all of the lines analysed so far have leaves with increased cell layers in the leaf mesophyll and elongated palisade cells.

### Genetic Resources

The following genetic resources were used in the making of the invention:

Seeds of Arabidopsis thaliana col-0 were obtained from the seed collection at Rothamsted Research, Rothamsted Research, West Common, Harpenden, Hertfordshire, AL5 2JQ, United Kingdom.

Seeds of Camelina sativa ‘Celine’ were obtained the seed collection at Rothamsted Research, Rothamsted Research, West Common, Harpenden, Hertfordshire, AL5 2JQ, United Kingdom.

Wild type hybrid poplar (Populus tremula X Populus alba) Institut National de la Recherche Agronomique (INRA) clone 7171-B was obtained from the University of Malaga, Spain.

### Nucleotide and Amino Acid Sequences

Arabidopsis thaliana IQD22 protein (AT4G23060.1)

[SEQ ID NO: 1]

| | |
|---|---|
| 1 | MGKASRWFRS LFGVKKPDPG YPDLSVETPS RSTSSNLKRR |
| | WSFVKSKREK |
| 51 | ESTPINQVPH TPSLPNSTPP PPSHHQSSPR RRRKQKPMWE |
| | DEGSEDSDKH |
| 101 | AIAVAAATAA VAEAAVAAAN AAAAVVRLTS TSGRSTRSPV |
| | KARFSDGFDD |
| 151 | VVAHGSKFYG HGRDSCELAV IKIQSIFRGY LAKRALRALK |
| | GLVRLQAIVR |
| 201 | GHIERKRMSV HLRRMHALVR AQARVRATRV IVTPESSSSQ |
| | SNNTKSSHFQ |
| 251 | NPGPPTPEKL EHSISSRSSK LAHSHLFKRN GSKASDNNRL |
| | YPAHRETFSA |
| 301 | TDEEEKILQI DRKHISSYTR RNRPDMFYSS HLILDNAGLS |
| | EPVFATPFSP |
| 351 | SSSHEEITSQ FCTAENSPQL YSATSRSKRS AFTASSIAPS |
| | DCTKSCCDGD |
| 401 | HPSYMACTES SRAKARSASA PKSRPQLFYE RPSSKRFGFV |
| | DLPYCGDTKS |
| 451 | GPQKGSALHT SFMNKAYPGS GRLDRLGMPI GYRY |

Arabidopsis thaliana IQD22 full length genomic DNA. Underlined sequence portion is the coding region. The start codon is marked in bold:

[SEQ ID NO: 2]

| | |
|---|---|
| 1 | ATTTTACTTC CTCAGATTCA CATGACCAAA TCATGTAACC |
| | ATTTTCCAAA |
| 51 | TAAAATCTTT ACATTTAGAT TTAGATTCAG AGGAATTGAA |
| | TTAGCCTCAT |
| 101 | CATAATGTAT GATACTACAT ACTACACTAG TGACACGGAA |
| | ATTACACGAC |
| 151 | GAAAATAAAC AATGTGAAGA ATAACGAAAT TTCCCGGGAA |
| | AAGAGAGATA |
| 201 | GAGAGTGAGA CACACGCGCG AGTGATGCGT GTGGTAGTAA |
| | ATAGAACACT |
| 251 | GTTTGATGAT ACTGCTGCGA CTACTTAACT CTTATTACAA |
| | AGCTCTCTTT |
| 301 | TTGTTGTCTC TCTCTTGCTC TCTCTGCAAA ACTCCGGCGA |
| | GAAGAGAACG |
| 351 | TTGTCGTTTC ATTCGTATCA AAGTCTTCAT CAGCA |
| | ATGGG AAAAGCGTCA |
| 401 | CGGTGGTTTA GGAGTCTATT CGGAGTTAAG AAACCCGACC |
| | CGGGTTATCC |
| 451 | GGATCTATCC GTCGAGACGC CTTCTCGGTC AACTTCCTCT |
| | AATCTCAAAC |
| 501 | GCCGATGGAG TTTCGTCAAA TCCAAACGAG AAAAAGAAAG |
| | TACACCGATT |
| 551 | AATCAAGTTC CTCATACTCC ATCGCTACCG AATTCGACGC |
| | CTCCACCGCC |
| 601 | GTCTCACCAC CAATCGTCGC CGAGACGACG GAGAAAACAA |
| | AAGCCGATGT |
| 651 | GGGAGGATGA GGGAAGTGAA GATTCGGACA AGCATGCTAT |
| | TGCGGTGGCT |
| 701 | GCCGCGACTG CTGCGGTTGC TGAAGCTGCA GTCGCCGCCG |
| | CTAATGCTGC |
| 751 | TGCTGCGGTC GTCAGGCTGA CGAGCACAAG TGGGAGGTCG |
| | ACTCGAAGTC |
| 801 | CTGTTAAGGC ACGGTTTAGC GACGGATTCG ACGACGTGGT |
| | GGCGCATGGT |
| 851 | AGCAAGTTTT ATGGACACGG CCGTGACAGT TGTGAACTTG |
| | CGGTGATTAA |
| 901 | GATACAATCT ATATTTCGCG GATACTTG GT AATTTTCTTT |
| | CTCTAAAATT |
| 951 | TATTTCACTC GTTTTATAAA TTACTTTATG AAGCCAAAAA |
| | TAGAAATATA |
| 1001 | CATGAATTGC ATTTTTTTAG TCAAAAGTTT ATAATTTTTG |
| | ATTAAACGAA |
| 1051 | AGCTGAATGA ATACATTTGT GTTCATCATT TACACGATTT |
| | TGCATAATTA |
| 1101 | TATTTTTAAT TAGGATTAGA AAAGTATAAA ATGGTTATAG |
| | ATTTGTTAAA |
| 1151 | CATTCACGGG GATTATCGTC CAGATAACAT TTTTCGATGT |
| | CGTAAACCTT |
| 1201 | CTGCTGCTAC CACTTCCCTA GACGAGTGAT AGGACAATTA |
| | ACCTTCCATA |
| 1251 | TTCTCATTTA ATTATTTTAA TATCTTAGCA GAGCACTTGA |
| | ATATTCTTTG |
| 1301 | TAAAGTAATT TGCTTTGTGA ATACCAAATC AAATTGTATT |
| | TGCTTTTTCC |
| 1351 | TTAATTTTAT ATTCACTGGT TAGAAATTTG TTTTTCAATT |
| | TAACCGACTC |
| 1401 | AAAAATTGAT TCCGGTCCGA ACAGTATAGA TGATTCTCAA |
| | CAAGTTTAAT |
| 1451 | GCTAAGTTTA ATATTATATC CATTCTTCCA ATTAATTCGA |
| | TCCTCAACAA |
| 1501 | GTTTAATGCT AAGTTGTTTT CTTTTTTTTT TTTCTTTTAG |
| | TTTCCCCATT |
| 1551 | TGGTTACGAG TAAATGAAAG ATTTGTATAA AAATACTTAG |
| | TGGAAAATCA |
| 1601 | GTTAACATCA ACCAATACGA AGAAGTGGTC GTACTCTTGT |
| | TCTATTGGTT |
| 1651 | AGTGAATGTT ATTCACTGGA CCACTGTATA AATAACATGG |
| | TGAATGCTGG |
| 1701 | CTGCTATAAT TAGTCTATAT TTCAGTTCAT TCTCTATATG |
| | TTATAGTCTC |
| 1751 | TATGTAGTGA ATATACTATG TCTATTAATG TTTCGATGCA |
| | TGCCTATTGA |
| 1801 | AACTTTATAC TTTATAGCTT ATGGCGAATC TTACAAAAAA |
| | AAAATTTTGG |
| 1851 | AGGGGTTTTA AGATAATAGT GTGTAAGACA ATTCACACCC |
| | AAATGTGTAC |
| 1901 | TCATCGACAT AAGTCGCATT GTTCATTAAT AGTCGATTCA |
| | TCTTTTCTCT |
| 1951 | CGTAGAGAGT GACTATACAT GTAAATGTTT TTTTTATATA |
| | TGTCCCTCTA |
| 2001 | TTTAATTTTG TTGAATTCCT CGTGAATATT TCAAAAGTTC |
| | AATTTCTTTA |
| 2051 | AAAGGTCAAT ATGTTTTCTT TCTTTTTCCC TTTCTTTTGG |
| | CGCCAACTTA |
| 2101 | TATCAAAAGT AATGCTTTTT CAAATTTCAA TATGCTTTTG |
| | TGGTCCTGTC |
| 2151 | CATAATGAGA GCTTGAGCTA GTCAAGACTC TTAATGTTTT |
| | TTTAAGAGTT |
| 2201 | GATACTTAGA ATATGATCTT GTGATTATTG GCTGGCTTAT |
| | CTCCATTTCT |
| 2251 | AAGGCAATTT GTTATTGTTT GATTATTTTA ATTGTAG |
| | GCA AAGAGAGCGT |
| 2301 | TAAGGGCACT CAAGGGTTTG GTTAGGCTTC AAGCGATAGT |
| | TAGAGGCCAT |
| 2351 | ATCGAAAGAA AGAGAATGTC AGTCCATCTG CGCAGGATGC |
| | ACGCTTTGGT |
| 2401 | TCGAGCTCAG GCTCGTGTGC GTGCCACTCG GGTTATTGTC |
| | ACGCCTGAAT |
| 2451 | CTTCTTCTTC TCAATCCAAC AATACCAAAT CTTCTCACTT |
| | CCAAAACCCT |
| 2501 | GTAAGCCTTG TAAAATTCCC TATGATTGTT CCTTTTAACC |
| | TAAAACATGT |
| 2551 | CTTTTCGTTC AAGGTCCCTT TTTAACAACT CCTCTGTTTT |
| | TCTTTAAG GG |
| 2601 | TCCACCAACT CCGGAAAAAC TCGAGCATTC GATCTCTTCT |
| | CGCAGCTCCA |
| 2651 | AACTCGCTCA TTCTCATCTT TTCAAG GTAT TACATTTTCA |
| | GTTGCTGTTT |
| 2701 | GTTTCTTCTG TTTTTGTTGC TTGTGGTCCC ATTAGTTCAA |
| | AATTTCAGAG |
| 2751 | ACTATACAAG TTACTGACAT TGCTTTATGT TCAAAACAAA |
| | TCCAATCTCA |
| 2801 | AGAATTGGTT TATAGGCTTC TCATTGTTCC TTTGTTAGAC |
| | TTATCCATTT |
| 2851 | CACATTCCAT TTAAACTAAG AGATTGGATT AAATTTTCAG |
| | AGGAATGGTT |
| 2901 | CGAAGGCAAG CGACAACAAC AGACTGTACC CTGCTCACAG |
| | GGAGACATTC |
| 2951 | TCAGCCACAG ACGAAGAAGA AAAGATTCTT CAAATCGACA |
| | GGAAACACAT |
| 3001 | CAGTTCTTAC ACAAGACGCA ACAGACCAGA CATGTTCTAC |
| | TCATCCCACC |
| 3051 | TCATCCTAGA CAATGCTGGC CTGTCTGAAC CAGTTTTCGC |
| | CACGCCTTTT |
| 3101 | AGCCCGTCCT CGTCGCATGA AGAGATTACA AGCCAGTTTT |
| | GCACTGCAGA |
| 3151 | GAACAGTCCT CAGTTATACT CAGCTACTTC TAGAAGCAAA |
| | CGCAGTGCTT |
| 3201 | TCACCGCTAG TTCTATAGCA CCGAGCGATT GCACAAAAAG |
| | CTGCTGTGAT |
| 3251 | GGTGACCATC CAAGCTACAT GGCTTGTACA GAGTCCTCTA |
| | GGGCTAAGGC |
| 3301 | TAGGTCCGCT AGTGCCCCGA AGTCTCGACC ACAGTTATTT |
| | TACGAGCGGC |
| 3351 | CTTCATCAAA ACGGTTTGGA TTTGTTGATT TGCCGTACTG |
| | TGGTGATACA |
| 3401 | AAGTCCGGTC CCCAGAAAGG CTCTGCTCTG CATACTAGTT |
| | TTATGAACAA |
| 3451 | GGCTTATCCC GGTTCAGGTC GGTTGGACCG TCTCGGGATG |
| | CCAATTGGGT |
| 3501 | ATAGGTACTG A GAACGTTGT TGGACCCTTT AACCTGGTTT |
| | TGTTGTAGAT |
| 3551 | CAGTGGATTG AGCTTTGCTT CTAATTTTCT TTGTTTGTAG |
| | TTTGGAGCTA |
| 3601 | AGTGGATTTA ATCTAAAATG CAAATAGAAC TTGCTTAATC |
| | AAAATGTTTG |
| 3651 | CATCTCTATA AAGCATTGTC ATTTGCCTAG ACTTTAGTTG |
| | TTATCAAAAA |
| 3701 | TTTTATGAAT ATGAAGGAAA CCAAGAGCTA TAACAGATAT |
| | ATTTGGTGTA |
| 3751 | ATAACAAAAA CGTCTAACAC TAAAAAAATT GACAAGAAAA |
| | TGTCATGTCC |
| 3801 | ATTGGGATTT AGAGGAG |

Arabidopsis thaliana IQD22 Full Length cDNA:

[SEQ ID NO: 3]

| | |
|---|---|
| 1 | ATTTTACTTC CTCAGATTCA CATGACCAAA TCATGTAACC |
| | ATTTTCCAAA |
| 51 | TAAAATCTTT ACATTTAGAT TTAGATTCAG AGGAATTGAA |
| | TTAGCCTCAT |
| 101 | CATAATGTAT GATACTACAT ACTACACTAG TGACACGGAA |
| | ATTACACGAC |
| 151 | GAAAATAAAC AATGTGAAGA ATAACGAAAT TTCCCGGGAA |
| | AAGAGAGATA |
| 201 | GAGAGTGAGA CACACGCGCG AGTGATGCGT GTGGTAGTAA |
| | ATAGAACACT |
| 251 | GTTTGATGAT ACTGCTGCGA CTACTTAACT CTTATTACAA |
| | AGCTCTCTTT |
| 301 | TTGTTGTCTC TCTCTTGCTC TCTCTGCAAA ACTCCGGCGA |
| | GAAGAGAACG |
| 351 | TTGTCGTTTC ATTCGTATCA AAGTCTTCAT CAGCAATGGG |
| | AAAAGCGTCA |
| 401 | CGGTGGTTTA GGAGTCTATT CGGAGTTAAG AAACCCGACC |
| | CGGGTTATCC |
| 451 | GGATCTATCC GTCGAGACGC CTTCTCGGTC AACTTCCTCT |
| | AATCTCAAAC |
| 501 | GCCGATGGAG TTTCGTCAAA TCCAAACGAG AAAAAGAAAG |
| | TACACCGATT |
| 551 | AATCAAGTTC CTCATACTCC ATCGCTACCG AATTCGACGC |
| | CTCCACCGCC |
| 601 | GTCTCACCAC CAATCGTCGC CGAGACGACG GAGAAAACAA |
| | AAGCCGATGT |
| 651 | GGGAGGATGA GGGAAGTGAA GATTCGGACA AGCATGCTAT |
| | TGCGGTGGCT |
| 701 | GCCGCGACTG CTGCGGTTGC TGAAGCTGCA GTCGCCGCCG |
| | CTAATGCTGC |
| 751 | TGCTGCGGTC GTCAGGCTGA CGAGCACAAG TGGGAGGTCG |
| | ACTCGAAGTC |
| 801 | CTGTTAAGGC ACGGTTTAGC GACGGATTCG ACGACGTGGT |
| | GGCGCATGGT |
| 851 | AGCAAGTTTT ATGGACACGG CCGTGACAGT TGTGAACTTG |
| | CGGTGATTAA |
| 901 | GATACAATCT ATATTTCGCG GATACTTGGC AAAGAGAGCG |
| | TTAAGGGCAC |
| 951 | TCAAGGGTTT GGTTAGGCTT CAAGCGATAG TTAGAGGCCA |
| | TATCGAAAGA |
| 1001 | AAGAGAATGT CAGTCCATCT GCGCAGGATG CACGCTTTGG |
| | TTCGAGCTCA |
| 1051 | GGCTCGTGTG CGTGCCACTC GGGTTATTGT CACGCCTGAA |
| | TCTTCTTCTT |
| 1101 | CTCAATCCAA CAATACCAAA TCTTCTCACT TCCAAAACCC |
| | TGGTCCACCA |
| 1151 | ACTCCGGAAA AACTCGAGCA TTCGATCTCT TCTCGCAGCT |
| | CCAAACTCGC |
| 1201 | TCATTCTCAT CTTTTCAAGA GGAATGGTTC GAAGGCAAGC |
| | GACAACAACA |
| 1251 | GACTGTACCC TGCTCACAGG GAGACATTCT CAGCCACAGA |
| | CGAAGAAGAA |
| 1301 | AAGATTCTTC AAATCGACAG GAAACACATC AGTTCTTACA |
| | CAAGACGCAA |
| 1351 | CAGACCAGAC ATGTTCTACT CATCCCACCT CATCCTAGAC |
| | AATGCTGGCC |
| 1401 | TGTCTGAACC AGTTTTCGCC ACGCCTTTTA GCCCGTCCTC |
| | GTCGCATGAA |
| 1451 | GAGATTACAA GCCAGTTTTG CACTGCAGAG AACAGTCCTC |
| | AGTTATACTC |
| 1501 | AGCTACTTCT AGAAGCAAAC GCAGTGCTTT CACCGCTAGT |
| | TCTATAGCAC |
| 1551 | CGAGCGATTG CACAAAAAGC TGCTGTGATG GTGACCATCC |
| | AAGCTACATG |
| 1601 | GCTTGTACAG AGTCCTCTAG GGCTAAGGCT AGGTCCGCTA |
| | GTGCCCCGAA |
| 1651 | GTCTCGACCA CAGTTATTTT ACGAGCGGCC TTCATCAAAA |
| | CGGTTTGGAT |
| 1701 | TTGTTGATTT GCCGTACTGT GGTGATACAA AGTCCGGTCC |
| | CCAGAAAGGC |
| 1751 | TCTGCTCTGC ATACTAGTTT TATGAACAAG GCTTATCCCG |
| | GTTCAGGTCG |
| 1801 | GTTGGACCGT CTCGGGATGC CAATTGGGTA TAGGTACTGA |
| | GAACGTTGTT |
| 1851 | GGACCCTTTA ACCTGGTTTT GTTGTAGATC AGTGGATTGA |
| | GCTTTGCTTC |
| 1901 | TAATTTTCTT TGTTTGTAGT TTGGAGCTAA GTGGATTTAA |
| | TCTAAAATGC |
| 1951 | AAATAGAACT TGCTTAATCA AAATGTTTGC ATCTCTATAA |
| | AGCATTGTCA |
| 2001 | TTTGCCTAGA CTTTAGTTGT TATCAAAAAT TTTATGAATA |
| | TGAAGGAAAC |
| 2051 | CAAGAGCTAT AACAGATATA TTTGGTGTAA TAACAAAAAC |
| | GTCTAACACT |
| 2101 | AAAAAAATTG ACAAGAAAAT GTCATGTCCA TTGGGATTTA |
| | GAGGAG |

Arabidopsis thaliana IQD23 Protein (AT5G62070.1)

[SEQ ID NO: 4]

| | |
|---|---|
| 1 | MGFFGRLFGS KKKSDKAASS RDKRRWSFTT RSSNSSKRAP |
| | AVTSASVVEQ |
| 51 | NGLDADKHAI AVAAATAAVA EAALTAAHAA AEVVRLTSGN |
| | GGRNVGGGGN |
| 101 | SSVFQIGRSN RRWAQENIAA MKIQSAFRGY LARRALRALK |
| | ALVKLQALVR |
| 151 | GHIVRKQTAD MLRRMQTLVR LQSQARARAS RSSHSSASFH |
| | SSTALLFPSS |
| 201 | SSSPRSLHTR CVSNAEVSSL DHRGGSKRLD WQAEESENGD |
| | KILEVDTWKP |
| 251 | HYHPKPLRSE RNNESPRKRQ QSLLGPRSTE NSPQVGSSGS |
| | RRRTPFTPTS |
| 301 | RSEYSWGCNN YYYSGYHPNY MANTESYKAK VRSQSAPKQR |
| | VEVSNETSGY |
| 351 | KRSVQGQYYY YTAVEEESLD VGSAGYYGGG GGDSDRLNRN |
| | QSAKSRMHSS |
| 401 | FLV |

Arabidopsis thaliana IQD23 Full Length Genomic DNA:

[SEQ ID NO: 5]

| | |
|---|---|
| 1 | AAAATAAAAA AGCCTTTTAA AATCTCGTGT AACTAACTCG |
| | CTGTATGCCG |
| 51 | GCGCGTGGAA ACGTACTCTG CAATCTATAT ATAAGCCGTT |
| | AAACTCTATT |
| 101 | TAGCTTCTCT ATAGACAAAA GTTTATGTTT TGTTTTTCCT |
| | CTAACACAAA |
| 151 | AAACAATCTC AGATATTAAA TAAAGTGTTA ATCAACCGGA |
| | GAACTCTCCG |
| 201 | ACAAAACCTC GCCGACGATT TTCCGACGAT GAAAGATGGG |
| | CTTTTTCGGG |
| 251 | AGACTGTTCG GGAGTAAAAA AAAGTCTGAT AAAGCTGCTT |
| | CGTCGAGAGA |
| 301 | TAAACGGAGG TGGAGCTTCA CCACCAGATC TTCAAATTCC |
| | AGCAAGAGAG |
| 351 | CTCCGGCGGT GACGTCGGCT TCGGTGGTTG AGCAAAATGG |
| | TTTGGATGCT |
| 401 | GACAAACATG CGATAGCTGT GGCTGCTGCG ACAGCCGCGG |
| | TGGCGGAAGC |
| 451 | AGCTCTTACT GCTGCTCATG CGGCAGCTGA AGTCGTGAGA |
| | CTAACAAGCG |
| 501 | GAAACGGCGG GAGAAACGTC GGTGGCGGTG GAAACTCGTC |
| | GGTGTTTCAA |
| 551 | ATAGGAAGAA GTAACCGTCG GTGGGCTCAG GAGAATATCG |
| | CGGCGATGAA |
| 601 | GATTCAATCC GCTTTTCGTG GTTATCTGGT TAGTTTACTA |
| | ATTTCTCAAA |
| 651 | CGTTTTAGCT GATTAATTCA AGAATGATTC AAAATGTTGA |
| | ATTTTGAAAC |
| 701 | TGTTCGAAAA TTTCATAAAA CAATTTACAT AATGTGCAAT |
| | AACTGTTGAA |
| 751 | ATTCGGATTT TTTTTCTTCT CATTCACAAT TTCAAATTTT |
| | GAAAATGGAA |
| 801 | ACAATGGGTA AAACTTGTAT TGTTTTGTGT GGGGTTTGGT |
| | CCCAAAAGAA |
| 851 | ACTGACTATA GAAGAAGAAG AAGAAGACAA ACAAATGTGA |
| | AATGAAGAAG |
| 901 | GTTCTTTGAA ACTTGTACAC TCTGCCGACA CTATTTTTAG |
| | TGGTTATCAA |
| 951 | TAGTTCCAGT TTTTTTTTTT ATCTTCTTCT GTTAGTTTAT |
| | CACTCAAAAT |
| 1001 | TTTGATGAAA GATCTGATCA GAATGTCGAA ATGAACTTAG |
| | TTTAATGTTG |
| 1051 | GTGTGAATCA TTTGATTTTA GTTGTCAGAT GGGCCAAGTT |
| | TTTTTTTGTT |
| 1101 | CCACGTTATG TTGTGCTTTA TAATTCTGAA CCTTTATTGA |
| | AATTAACAAG |
| 1151 | ACTAATTAGT ATTTTACAGA TGGGTTCTTT TTTGCTTTAG |
| | GACAGCAACT |
| 1201 | TTGAAGTTGA AATCTTTAGC CCAAAAACTT TTTCAAGTTG |
| | CTTATTTATC |
| 1251 | TTCTCTTTAC TTACAAAGAT TCATTTTGAC CATGTTTTTT |
| | ACTCTTTTTC |
| 1301 | AAATTCCTAT CTGTGAATCT TGGAGTTTTT TTTGTTTTTG |
| | CTAATTTGCG |
| 1351 | TTAAATTATT CCAAGATCTT ATGTGTGTTG TCGAATCTTG |
| | ACCAATGTGT |
| 1401 | AAAGAAGAAC TCGTGTGTGT TTGTGTGTGT ACATTTGACT |
| | TTGAGACATA |
| 1451 | GTTATGAGAA TGGTGGGCTC TGTTGTAATT ACTAAATCTA |
| | GTTTTTGGTA |
| 1501 | CAAGTGGAAG TGGTTGAGAA ACTAAAAGCT AAATAGAATA |
| | ATTTCACAAA |
| 1551 | CAACTAATCC CTTTACTACT CTTTTTCTGT GTGTTTTAAT |
| | GTTAGTTGGC |
| 1601 | AAAACGATGA TTATGTCTTC AACAAATGAT TGAACTCGCC |
| | TTGTCTTTGT |
| 1651 | TATTCACTTT CAAGCAAACT CAAATTTTGA TTATTCATGG |
| | TGGAGCCAAT |
| 1701 | TTTTTACTCC AAGTTCTTTC TATATGGAGT AAATATTCAT |
| | TTTTTGAACA |
| 1751 | TAAATTAAAG ACTCTATATG AAGTTTATTG GTTTGCTTTG |
| | GCTACTTTGG |
| 1801 | AAAATTTTCA GGCGAGGAGA GCATTACGAG CACTAAAGGC |
| | ATTAGTGAAG |
| 1851 | CTTCAAGCAT TAGTGAGAGG ACACATAGTG AGAAAACAAA |
| | CTGCAGATAT |
| 1901 | GCTTAGAAGG ATGCAGACTC TTGTTCGTCT TCAATCTCAA |
| | GCTCGTGCTC |
| 1951 | GAGCCTCTCG TTCTTCTCAC TCCTCTGCTT CTTTCCACTC |
| | CTCCACCGCT |
| 2001 | CTTTTGTTCC CATCTTCCTC GTCTTCTCCA CGTTCTCTTC |
| | ACACGCGCTG |
| 2051 | CGTTTCAAAC GCTGAAGTCA GCTCTCTTGA CCACCGTGGA |
| | GGCTCTAAGC |
| 2101 | GGTTAGATTG GCAGGCGGAG GAAAGCGAAA ATGGAGACAA |
| | GATCCTAGAA |
| 2151 | GTGGATACTT GGAAGCCTCA TTATCATCCC AAACCGTTAC |
| | GTTCAGAGAG |
| 2201 | AAACAATGAG TCTCCGAGGA AACGACAACA ATCTTTGTTG |
| | GGTCCGAGAA |
| 2251 | GTACAGAGAA TAGTCCTCAA GTTGGTTCTA GTGGGTCAAG |
| | AAGAAGAACT |
| 2301 | CCTTTTACGC CGACGTCAAG AAGCGAGTAT TCTTGGGGAT |
| | GTAATAACTA |
| 2351 | TTACTACTCG GGTTATCACC CGAATTACAT GGCTAACACT |
| | GAGTCTTATA |
| 2401 | AAGCTAAAGT TCGGTCACAA AGTGCGCCGA AACAGAGAGT |
| | TGAGGTCTCT |
| 2451 | AATGAGACCA GTGGCTACAA GAGATCTGTT CAGGGACAGT |
| | ATTACTACTA |
| 2501 | CACAGCGGTA GAAGAAGAGA GTTTGGATGT TGGAAGCGCT |
| | GGTTACTACG |
| 2551 | GAGGAGGAGG AGGCGATTCT GATCGATTGA ATCGGAACCA |
| | AAGTGCGAAA |
| 2601 | TCGAGGATGC ATTCTTCGTT TCTTGTTTAG ATTGTGATTC |
| | TCTTTCTCTT |
| 2651 | CTTTTTTTTG GTTTGAGTTT GGTAATTTTC CAGAAGAGAT |
| | AAGTATTAAC |
| 2701 | ACTGGATGTG TAAATTGTTG TTAACAAGTT TCGATTGCTT |
| | GCAAAAGAAG |
| 2751 | GAACACCACT CTGTTCTCTA TCTTTGTTGA TTAGTGTTCA |
| | ATTAAGATTA |
| 2801 | ATCAAACTGA GTCAAACCCC ACAATTTA |

Arabidopsis thaliana IQD23 Full Length cDNA

[SEQ ID NO: 6]

| | |
|---|---|
| 1 | AAAATAAAAA AGCCTTTTAA AATCTCGTGT AACTAACTCG |
| | CTGTATGCCG |
| 51 | GCGCGTGGAA ACGTACTCTG CAATCTATAT ATAAGCCGTT |
| | AAACTCTATT |
| 101 | TAGCTTCTCT ATAGACAAAA GTTTATGTTT TGTTTTTCCT |
| | CTAACACAAA |
| 151 | AAACAATCTC AGATATTAAA TAAAGTGTTA ATCAACCGGA |
| | GAACTCTCCG |
| 201 | ACAAAACCTC GCCGACGATT TTCCGACGAT GAAAGATGGG |
| | CTTTTTCGGG |
| 251 | AGACTGTTCG GGAGTAAAAA AAAGTCTGAT AAAGCTGCTT |
| | CGTCGAGAGA |
| 301 | TAAACGGAGG TGGAGCTTCA CCACCAGATC TTCAAATTCC |
| | AGCAAGAGAG |
| 351 | CTCCGGCGGT GACGTCGGCT TCGGTGGTTG AGCAAAATGG |
| | TTTGGATGCT |
| 401 | GACAAACATG CGATAGCTGT GGCTGCTGCG ACAGCCGCGG |
| | TGGCGGAAGC |
| 451 | AGCTCTTACT GCTGCTCATG CGGCAGCTGA AGTCGTGAGA |
| | CTAACAAGCG |
| 501 | GAAACGGCGG GAGAAACGTC GGTGGCGGTG GAAACTCGTC |
| | GGTGTTTCAA |
| 551 | ATAGGAAGAA GTAACCGTCG GTGGGCTCAG GAGAATATCG |
| | CGGCGATGAA |
| 601 | GATTCAATCC GCTTTTCGTG GTTATCTGGC GAGGAGAGCA |
| | TTACGAGCAC |
| 651 | TAAAGGCATT AGTGAAGCTT CAAGCATTAG TGAGAGGACA |
| | CATAGTGAGA |
| 701 | AAACAAACTG CAGATATGCT TAGAAGGATG CAGACTCTTG |
| | TTCGTCTTCA |
| 751 | ATCTCAAGCT CGTGCTCGAG CCTCTCGTTC TTCTCACTCC |
| | TCTGCTTCTT |
| 801 | TCCACTCCTC CACCGCTCTT TTGTTCCCAT CTTCCTCGTC |
| | TTCTCCACGT |
| 851 | TCTCTTCACA CGCGCTGCGT TTCAAACGCT GAAGTCAGCT |
| | CTCTTGACCA |
| 901 | CCGTGGAGGC TCTAAGCGGT TAGATTGGCA GGCGGAGGAA |
| | AGCGAAAATG |
| 951 | GAGACAAGAT CCTAGAAGTG GATACTTGGA AGCCTCATTA |
| | TCATCCCAAA |
| 1001 | CCGTTACGTT CAGAGAGAAA CAATGAGTCT CCGAGGAAAC |
| | GACAACAATC |
| 1051 | TTTGTTGGGT CCGAGAAGTA CAGAGAATAG TCCTCAAGTT |
| | GGTTCTAGTG |
| 1101 | GGTCAAGAAG AAGAACTCCT TTTACGCCGA CGTCAAGAAG |
| | CGAGTATTCT |
| 1151 | TGGGGATGTA ATAACTATTA CTACTCGGGT TATCACCCGA |
| | ATTACATGGC |
| 1201 | TAACACTGAG TCTTATAAAG CTAAAGTTCG GTCACAAAGT |
| | GCGCCGAAAC |
| 1251 | AGAGAGTTGA GGTCTCTAAT GAGACCAGTG GCTACAAGAG |
| | ATCTGTTCAG |
| 1301 | GGACAGTATT ACTACTACAC AGCGGTAGAA GAAGAGAGTT |
| | TGGATGTTGG |
| 1351 | AAGCGCTGGT TACTACGGAG GAGGAGGAGG CGATTCTGAT |
| | CGATTGAATC |
| 1401 | GGAACCAAAG TGCGAAATCG AGGATGCATT CTTCGTTTCT |
| | TGTTTAGATT |
| 1451 | GTGATTCTCT TTCTCTTCTT TTTTTTGGTT TGAGTTTGGT |
| | AATTTTCCAG |
| 1501 | AAGAGATAAG TATTAACACT GGATGTGTAA ATTGTTGTTA |
| | ACAAGTTTCG |
| 1551 | ATTGCTTGCA AAAGAAGGAA CACCACTCTG TTCTCTATCT |
| | TTGTTGATTA |
| 1601 | GTGTTCAATT AAGATTAATC AAACTGAGTC AAACCCCACA |
| | ATTTA |

Arabidopsis thaliana IQD24 Protein (AT5G07240.1)

[SEQ ID NO: 7]

| | |
|---|---|
| 1 | MGFFGRLFGS KKQEKATPNR RRWSFATRSS HPENDSSSHS |
| | SKRRGDEDVL |
| 51 | NGDKHAIAVA AATAAVAEAA LAAARAAAEV VRLTNGGRNS |
| | SVKQISRSNR |
| 101 | RWSQEYKAAM KIQSAFRGYL ARRALRALKA LVKLQALVKG |
| | HIVRKQTADM |
| 151 | LRRMQTLVRL QARARASRSS HVSDSSHPPT LMIPSSPQSF |
| | HARCVSEAEY |
| 201 | SKVIAMDHHH NNHRSPMGSS RLLDQWRTEE SLWSAPKYNE |
| | DDDKILEVDT |
| 251 | WKPHFRESPR KRGSLVVPTS VENSPQLRSR TGSSSGGSRR |
| | KTPFTPARSE |
| 301 | YEYYSGYHPN YMANTESYKA KVRSQSAPRQ RLQDLPSESG |
| | YKRSIQGQYY |
| 351 | YYTPAAERSF DQRSDNGIAG YRGVSDGLDR NQSDKSKMYT |
| | SFFSSNPLFF |
| 401 | Q |

Arabidopsis thaliana IQD24 Full Length DNA

[SEQ ID NO: 8]

| | |
|---|---|
| 1 | TTCTCCATTC CCAATTGTCT CTTCTTTTCT TTTTGTACTT |
| | GTCAAAAACA |
| 51 | AAAAGAACAA CAAAAAAAAT CTCAACCGTA GAAAATTCCG |
| | ACAAGAGTTC |
| 101 | AGTTCATACA ATGAACTAAG TATGGGTTTC TTTGGAAGAC |
| | TGTTCGGAAG |
| 151 | TAAGAAGCAA GAAAAGGCAA CACCGAACAG ACGAAGATGG |
| | AGCTTCGCTA |
| 201 | CTAGATCCTC ACATCCCGAG AATGATTCGT CTTCTCATTC |
| | AAGCAAGAGA |
| 251 | CGTGGGGATG AAGATGTCTT AAACGGCGAC AAGCATGCGA |
| | TAGCCGTCGC |
| 301 | GGCTGCTACA GCTGCAGTGG CTGAAGCCGC ACTCGCTGCT |
| | GCTCGTGCGG |
| 351 | CGGCGGAAGT CGTGAGACTC ACCAATGGTG GTAGAAACTC |
| | GTCGGTAAAA |
| 401 | CAAATCAGTC GGAGTAATCG TCGGTGGTCT CAAGAGTATA |
| | AAGCAGCTAT |
| 451 | GAAGATTCAA TCCGCTTTTC GTGGCTACTT GGTGAGTTAA |
| | TTACTTCTCT |
| 501 | AAGTTTAATC TTAATACTCA ACTTCATAAT CTATAATTTT |
| | GTTTGAAAGA |
| 551 | AATTAAGTTT TTGAGCCAAA GTGTGGAATA GCTGTCTGAT |
| | GTTGATTTTT |
| 601 | CTACTTAAAC CCAAATTGAA ATATTTACTA AACAAAAAAA |
| | AGAAAGAATG |
| 651 | AGAATAATTT GTATTTATTT GTATTGGTCC CCGAAAAAGA |
| | CTAGGAAAGG |
| 701 | AGACAAATGA ATGAATAATG ATACCAAGAA ACTTGTTTAG |
| | AGCCGATACG |
| 751 | ATTCTCTACC GAGTACCGAC ACTATTTAAT GGTACTACTC |
| | TAGAATTGTA |
| 801 | TAGATAAAAT TTGGTCCCAA AAAATATGCA AATATTATCT |
| | CAAACAAAAT |
| 851 | CTTACCTAAC TAGATTTAGC TAAGGGCTTA TTCTTTGTAA |
| | TAAATTAATT |
| 901 | TTTACATTAA ACCAAGTCAG CAATAAAGAA TAACAAGGAG |
| | AGGGCCAAGT |
| 951 | TCTTTATTTT ATCTACTTTC TTTTTAGTTT CCAAAGTTCC |
| | AAAGTGTATT |
| 1001 | TTACTATAAA GGTGAAACTA CAGATTGGTT TTTAAGCTTT |
| | TTGAGCTTTA |
| 1051 | AAGTTGCATC ACCACTGTTA CTATCAAAGT TGCAACCTAC |
| | CTTTTTCATA |
| 1101 | ATTTGACAGA TAGGTTATGT AGTTTAGACA CATAATTTAG |
| | AGATTCTCTA |
| 1151 | ATCTTATTGC TTCGTATATA ACAAATAAGG TGTGTATCAT |
| | TTAGAAGAAA |
| 1201 | ATGATAGTGG TTGGGAAACT ATGAGTAATT TAATAAGGAG |
| | TATTAGTGTA |
| 1251 | TTACTTTGTG GTTTGAATTA AGTGTGTAAC CAAGTTTTCT |
| | TTAATTCAGG |
| 1301 | CGAGGAGGGC GTTGAGAGCA CTGAAGGCAT TAGTGAAGCT |
| | TCAAGCGTTG |
| 1351 | GTGAAGGGAC ACATAGTAAG GAAACAAACG GCTGATATGC |
| | TGCGTCGAAT |
| 1401 | GCAAACGCTG GTTCGGCTCC AAGCACGAGC TAGAGCTTCG |
| | CGTTCTTCTC |
| 1451 | ACGTTTCTGA CTCTTCCCAT CCGCCAACAC TAATGATTCC |
| | ATCTTCCCCA |
| 1501 | CAATCTTTCC ATGCACGATG CGTTTCAGAG GCTGAGTACA |
| | GTAAAGTCAT |
| 1551 | TGCCATGGAT CACCACCACA ACAACCACCG TTCACCGATG |
| | GGTTCAAGCC |
| 1601 | GGTTATTAGA CCAATGGAGG ACAGAGGAAA GTCTATGGAG |
| | CGCACCAAAG |
| 1651 | TACAATGAAG ATGATGACAA AATCCTAGAA GTCGACACTT |
| | GGAAGCCTCA |
| 1701 | CTTCAGAGAG TCACCAAGGA AAAGAGGATC TCTAGTGGTT |
| | CCTACAAGTG |
| 1751 | TGGAGAACAG TCCACAATTA AGGTCTAGAA CAGGAAGCAG |
| | CAGTGGTGGT |
| 1801 | TCAAGGAGAA AAACTCCCTT CACGCCTGCG AGAAGCGAGT |
| | ACGAGTACTA |
| 1851 | CTCTGGGTAT CACCCTAACT ACATGGCTAA CACTGAGTCT |
| | TACAAAGCAA |
| 1901 | AAGTCCGATC ACAAAGCGCA CCAAGACAGA GACTACAAGA |
| | TTTACCTTCA |
| 1951 | GAGAGTGGTT ACAAGAGGTC TATACAGGGA CAGTATTACT |
| | ACTACACGCC |
| 2001 | TGCTGCAGAG CGATCGTTTG ATCAGCGTTC GGATAACGGG |
| | ATCGCGGGTT |
| 2051 | ACAGAGGAGT TTCTGATGGG TTAGATCGAA ACCAAAGTGA |
| | CAAATCGAAG |
| 2101 | ATGTACACTT CGTTTTTCAG TTCTAATCCT CTTTTCTTTC |
| | AATAGTCGAG |
| 2151 | AAAGGATGAA AAAAGTGAGT GGAATGTGTA AAATTAGATT |
| | TCGACACACG |
| 2201 | AGTACAGAGA CAGCCAGTGA TCAATCTGTG TTTTGTACTA |
| | TTTTCTAATT |
| 2251 | GACTGTATCC AACAAGGGTC CATTCTTGTC TGATAAAAAA |
| | ACTTCAATAA |
| 2301 | TTTGAAGTGA TGTCAAGTCA AGACGTGGGA ATCACCACTT |
| | AAAGCAATGA |
| 2351 | AAATTGATTG ATATAACCTT TCATATTAAA CT |

Arabidopsis thaliana IQD24 cDNA

[SEQ ID NO: 9]

| | |
|---|---|
| 1 | TTCTCCATTC CCAATTGTCT CTTCTTTTCT TTTTGTACTT GTCAAAAACA |
| 51 | AAAAGAACAA CAAAAAAAAT CTCAACCGTA GAAAATTCCG ACAAGAGTTC |
| 101 | AGTTCATACA ATGAACTAAG TATGGGTTTC TTTGGAAGAC TGTTCGGAAG |
| 151 | TAAGAAGCAA GAAAAGGCAA CACCGAACAG ACGAAGATGG AGCTTCGCTA |
| 201 | CTAGATCCTC ACATCCCGAG AATGATTCGT CTTCTCATTC AAGCAAGAGA |
| 251 | CGTGGGGATG AAGATGTCTT AAACGGCGAC AAGCATGCGA TAGCCGTCGC |
| 301 | GGCTGCTACA GCTGCAGTGG CTGAAGCCGC ACTCGCTGCT GCTCGTGCGG |
| 351 | CGGCGGAAGT CGTGAGACTC ACCAATGGTG GTAGAAACTC GTCGGTAAAA |
| 401 | CAAATCAGTC GGAGTAATCG TCGGTGGTCT CAAGAGTATA AAGCAGCTAT |
| 451 | GAAGATTCAA TCCGCTTTTC GTGGCTACTT GGCGAGGAGG GCGTTGAGAG |
| 501 | CACTGAAGGC ATTAGTGAAG CTTCAAGCGT TGGTGAAGGG ACACATAGTA |
| 551 | AGGAAACAAA CGGCTGATAT GCTGCGTCGA ATGCAAACGC TGGTTCGGCT |
| 601 | CCAAGCACGA GCTAGAGCTT CGCGTTCTTC TCACGTTTCT GACTCTTCCC |
| 651 | ATCCGCCAAC ACTAATGATT CCATCTTCCC CACAATCTTT CCATGCACGA |
| 701 | TGCGTTTCAG AGGCTGAGTA CAGTAAAGTC ATTGCCATGG ATCACCACCA |
| 751 | CAACAACCAC CGTTCACCGA TGGGTTCAAG CCGGTTATTA GACCAATGGA |
| 801 | GGACAGAGGA AAGTCTATGG AGCGCACCAA AGTACAATGA AGATGATGAC |
| 851 | AAAATCCTAG AAGTCGACAC TTGGAAGCCT CACTTCAGAG AGTCACCAAG |
| 901 | GAAAAGAGGA TCTCTAGTGG TTCCTACAAG TGTGGAGAAC AGTCCACAAT |
| 951 | TAAGGTCTAG AACAGGAAGC AGCAGTGGTG GTTCAAGGAG AAAAACTCCC |
| 1001 | TTCACGCCTG CGAGAAGCGA GTACGAGTAC TACTCTGGGT ATCACCCTAA |
| 1051 | CTACATGGCT AACACTGAGT CTTACAAAGC AAAAGTCCGA TCACAAAGCG |
| 1101 | CACCAAGACA GAGACTACAA GATTTACCTT CAGAGAGTGG TTACAAGAGG |
| 1151 | TCTATACAGG GACAGTATTA CTACTACACG CCTGCTGCAG AGCGATCGTT |
| 1201 | TGATCAGCGT TCGGATAACG GGATCGCGGG TTACAGAGGA GTTTCTGATG |
| 1251 | GGTTAGATCG AAACCAAAGT GACAAATCGA AGATGTACAC TTCGTTTTTC |
| 1301 | AGTTCTAATC CTCTTTTCTT TCAATAGTCG AGAAAGGATG AAAAAAGTGA |
| 1351 | GTGGAATGTG TAAAATTAGA TTTCGACACA CGAGTACAGA GACAGCCAGT |
| 1401 | GATCAATCTG TGTTTTGTAC TATTTTCTAA TTGACTGTAT CCAACAAGGG |
| 1451 | TCCATTCTTG TCTGATAAAA AAACTTCAAT AATTTGAAGT GATGTCAAGT |
| 1501 | CAAGACGTGG GAATCACCAC TTAAAGCAAT GAAAATTGAT TGATATAACC |
| 1551 | TTTCATATTA AACT |

IQ67 Domain consensus portion
[SEQ ID NO: 10]
FRGYLARRALRALKGLVRLQALVRG
IQ67 Domain consensus
[SEQ ID NO: 11]
EE#AA#+IQX#FRGYLARRALRALKGLVRLQALVRGX#VR+QA##TL+CMQALVR#QAXVR
ARR#+#
>AT4G23060.1 IQD22
[SEQ ID NO: 12]
atgggaaaagcgtcacggtggtttaggagtctattcggagttaagaaacccgacccgggt
tatccggatctatccgtcgagacgccttctcggtcaacttcctctaatctcaaacgccga
tggagtttcgtcaaatccaaacgagaaaaagaaagtacaccgattaatcaagttcctcat
actccatcgctaccgaattcgacgcctccaccgccgtctcaccaccaatcgtcgccgaga
cgacggagaaaacaaaagccgatgtgggaggatgagggaagtgaagattcggacaagcat
gctattgcggtggctgccgcgactgctgcggttgctgaagctgcagtcgccgccgctaat
gctgctgctgcggtcgtcaggctgacgagcacaagtgggaggtcgactcgaagtcctgtt
aaggcacggtttagcgacggattcgacgacgtggtggcgcatggtagcaagttttatgga
cacggccgtgacagttgtgaacttgcggtgattaagatacaatctatatttcgcggatac
ttggcaaagagagcgttaagggcactcaagggtttggttaggcttcaagcgatagttaga
ggccatatcgaaagaaagagaatgtcagtccatctgcgcaggatgcacgctttggttcga
gctcaggctcgtgtgcgtgccactcgggttattgtcacgcctgaatcttcttcttctcaa
tccaacaataccaaatcttctcacttccaaaaccctggtccaccaactccggaaaaactc
gagcattcgatctcttctcgcagctccaaactcgctcattctcatcttttcaagaggaat
ggttcgaaggcaagcgacaacaacagactgtaccctgctcacagggagacattctcagcc
acagacgaagaagaaaagattcttcaaatcgacaggaaacacatcagttcttacacaaga
cgcaacagaccagacatgttctactcatcccacctcatcctagacaatgctggcctgtct
gaaccagttttcgccacgccttttagcccgtcctcgtcgcatgaagagattacaagccag
ttttgcactgcagagaacagtcctcagttatactcagctacttctagaagcaaacgcagt
gctttcaccgctagttctatagcaccgagcgattgcacaaaaagctgctgtgatggtgac
catccaagctacatggcttgtacagagtcctctagggctaaggctaggtccgctagtgcc
ccgaagtctcgaccacagttattttacgagcggccttcatcaaaacggtttggatttgtt
gatttgccgtactgtggtgatacaaagtccggtccccagaaaggctctgctctgcatact
agttttatgaacaaggcttatcccggttcaggtcggttggaccgtctcgggatgccaatt
gggtataggtactga
>AT5G62070.1 IQD23
[SEQ ID NO: 13]
atgggctttttcgggagactgttcgggagtaaaaaaaagtctgataaagctgcttcgtcg
agagataaacggaggtggagcttcaccaccagatcttcaaattccagcaagagagctccg
gcggtgacgtcggcttcggtggttgagcaaaatggtttggatgctgacaaacatgcgata
gctgtggctgctgcgacagccgcggtggcggaagcagctcttactgctgctcatgcggca
gctgaagtcgtgagactaacaagcggaaacggcgggagaaacgtcggtggcggtggaaac
tcgtcggtgtttcaaataggaagaagtaaccgtcggtgggctcaggagaatatcgcggcg
atgaagattcaatccgcttttcgtggttatctggcgaggagagcattacgagcactaaag
gcattagtgaagcttcaagcattagtgagaggacacatagtgagaaaacaaactgcagat
atgcttagaaggatgcagactcttgttcgtcttcaatctcaagctcgtgctcgagcctct
cgttcttctcactcctctgcttctttccactcctccaccgctcttttgttcccatcttcc
tcgtcttctccacgttctcttcacacgcgctgcgtttcaaacgctgaagtcagctctctt
gaccaccgtggaggctctaagcggttagattggcaggcggaggaaagcgaaaatggagac
aagatcctagaagtggatacttggaagcctcattatcatcccaaaccgttacgttcagag
agaaacaatgagtctccgaggaaacgacaacaatctttgttgggtccgagaagtacagag
aatagtcctcaagttggttctagtgggtcaagaagaagaactccttttacgccgacgtca
agaagcgagtattcttggggatgtaataactattactactcgggttatcacccgaattac
atggctaacactgagtcttataaagctaaagttcggtcacaaagtgcgccgaaacagaga
gttgaggtctctaatgagaccagtggctacaagagatctgttcagggacagtattactac
tacacagcggtagaagaagagagtttggatgttggaagcgctggttactacggaggagga
ggaggcgattctgatcgattgaatcggaaccaaagtgcgaaatcgaggatgcattcttcg
tttcttgtttag
AT5G07240.2 IQD24
[SEQ ID NO: 14]
atgtatcacactttagcttctccattcccaattgtctcttcttttctttttgtacttgtc
aaaaacaaaaagaacaacaaaaaaaatctcaaccgtagaaaattccgacaagagttcagt
tcatacaatgaactaagtatgggtttctttggaagactgttcggaagtaagaagcaagaa
aaggcaacaccgaacagacgaagatggagcttcgctactagatcctcacatcccgagaat
gattcgtcttctcattcaagcaagagacgtggggatgaagatgtcttaaacggcgacaag
catgcgatagccgtcgcggctgctacagctgcagtggctgaagccgcactcgctgctgct
cgtgcggcggcggaagtcgtgagactcaccaatggtggtagaaactcgtcggtaaaacaa
atcagtcggagtaatcgtcggtggtctcaagagtataaagcagctatgaagattcaatcc
gcttttcgtggctacttggcgaggagggcgttgagagcactgaaggcattagtgaagctt
caagcgttggtgaagggacacatagtaaggaaacaaacggctgatatgctgcgtcgaatg
caaacgctggttcggctccaagcacgagctagagcttcgcgttcttctcacgtttctgac
tcttcccatccgccaacactaatgattccatcttccccacaatctttccatgcacgatgc
gtttcagaggctgagtacagtaaagtcattgccatggatcaccaccacaacaaccaccgt
tcaccgatgggttcaagccggttattagaccaatggaggacagaggaaagtctatggagc
gcaccaaagtacaatgaagatgatgacaaaatcctagaagtcgacacttggaagcctcac
ttcagagagtcaccaaggaaaagaggatctctagtggttcctacaagtgtggagaacagt
ccacaattaaggtctagaacaggaagcagcagtggtggttcaaggagaaaaactcccttc
acgcctgcgagaagcgagtacgagtactactctgggtatcaccctaactacatggctaac
actgagtcttacaaagcaaaagtccgatcacaaagcgcaccaagacagagactacaagat
ttaccttcagagagtggttacaagaggtctatacagggacagtattactactacacgcct
gctgcagagcgatcgtttgatcagcgttcggataacgggatcgcgggttacagaggagtt
tctgatgggttagatcgaaaccaaagtgacaaatcgaagatgtacacttcgtttttcagt
tctaatcctcttttctttcaatag
Sequence of At4g23060 promoter used for construct of expressing IQD22 under the
control of its native promoter
[SEQ ID NO: 127]
ctttgccaacgaatgttagttgtgggggaaacaacaattccgctggtaattttgactgaattaaaaaattagagatagaatgcaa
ggagacacgtacgaacgagttcatacacgagaagcagttgaccagagtcaacaaacctgtaaaaagctaaagaaccacc
gtccactcgcagtctactatatacgactaactaagattcatgcaggctcaagtttttgtacttgtatgacacgtggctagatcgtaa
cggccgtttagactgatgtctcttcgtctctccgagtctttgtcctgtctttttctcattgcatgagacataccgaagattccagtggatt
aattgtcggtccatttcttttttcctttttactactgacttcgagttagattctctaaagtagtacacgtgaaccataggattcgtttatgatt
acataaaacctttgttagtagtgtttgttcatttcttaattctggttatacatttgaaacatatgcaaatttatcactcgacttattaatcct
aagttcgtaactaagttctttttggttaattagcttatatacttctcaagatttctgattctgacttatgctataaagtatatcttcgataagt
tgagtcgcatctctctcctcggaaaatattcttgtattttggacaaatttctcgtgtattgttttgatatttttgtggtcatgttgttttgtgaatt
agtatataagtagataaactacgtagttttcattcgtacaactatataaccaatcgtattcgacgaaataaatagtcagaaaatatt
ctatttgttatgcaataatatctcactatacagtccaaaatattaacctccatataaatctgatagtaacaacaaacaattttttctcttt
gtcctataacatacataatacatttatgtttgaaagacaagagaagaacaaaaacaagaacccacaaatgtcaaaatagtga
aagaattggattccgacttcgatttcccattacacaaaacgacgtagcaacggacggcgctagctattacgttaagagacgaa
aaacggagtagggaccaacaaaaaaacgtgatcatggatggattattaacgatagacattggattacggcgatggacccat
aaaactgacgtcatccgttatggggtccgtagtgtggtcctctaaaaagtattgacgctttttgtcatctctctgttctttaggactcgt
cacaaattttacttcctcagattcacatgaccaaatcatgtaaccattttccaaataaaatctttacatttagatttagattcagagga
attgaattagcctcatcataatgtatgatactacatactacactagtgacacggaaattacacgacgaaaataaacaatgtgaa
gaataacgaaatttcccgggaaaagagagatagagagtgagacacacgcgcgagtgatgcgtgtggtagtaaatagaac
actgtttgatgatactgctgcgactacttaactcttattacaaagctctctttttgttgtctctctcttgctctctctgcaaaactccggcg
agaagagaacgttgtcgtttcattcgt
start of gene (start codon underlined):
[SEQ ID NO: 128]
atcaaagtcttcatcagcaATGGGAAAAGCGTCACGG

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The readers attention is directed to all papers and documents which are filed concurrently with or previous to this specification in connection with this application and which are open to public inspection with this specification, and the contents of all such papers and documents are incorporated herein by reference.

What is claimed is:

1-46. (canceled)

47. A method of modifying a plant to increase yield compared to a corresponding unmodified plant, and/or whereby at least some of the leaves of the modified plant are: (i) thicker; and/or (ii) rounder in shape; and/or (iii) contain more chlorophyll per wet or dry weight, compared to a corresponding unmodified plant, comprising genetically modifying the plant to increase expression of a plant calmodulin-binding protein (IQD) compared to the unmodified plant grown under same or comparative conditions.

48. A method as claimed in claim 47, wherein the IQD protein is IQD22, IQD23 or IQD24.

49. A method as claimed in claim 47, wherein
(a) the IQD protein comprises a conserved 67 amino acid domain (IQ67) having an amino acid sequence with at least 50% identity to FRGYLARRALRALKGLVRLQALVRG [SEQ ID NO: 10] or at least 50% identity to EE#AA#IQX#FRGYLARRALRALKGLVRLQALVRGX#VR+QA##TL+CMQALVR#Q AXVRARR#+# [SEQ ID NO: 11]; and or
(b) wherein the protein further comprises the amino acid sequence: (i) HAIAVAAATAAVAEAA [SEQ ID NO: 15]; or (ii) (D/R/S/N)(K/Q/D/N/R/T)(H/R)(A/S)(I/V/M/S)(A/H)V(A/T)(A/E/F)(A/P)T(A/E)(A/H/V)(V/A) [SEQ ID NO: 16]; preferably DKHAIAVAAATAAV [SEQ ID NO: 17]; optionally further comprising the amino acid sequence: V(A/Q)(E/N/D/K)(A/N)(A/R)(V/Q/I/L)(A/T/N)(A/I)A(Q/E/V/N/H/R/N)AAA(A/V/E)VV(R/K/E/S/L/T)(L/F)(T/N)X [SEQ ID NO: 19], wherein X is any amino acid; optionally wherein this sequence is V(A/Q)(E/N/D/K)(A/N)(A/R)(V/Q/I/L)(A/T/N)(A/I)A(Q/E/V/N/H/R/N)AAA(A/V/E)VVRL TX [SEQ ID NO: 20] or
V(A/Q)(E/N/D/K)(A/N)(A/R)(V/Q/I/L)(A/T/N)(A/I)A(Q/E/V/N/H/R/N)AAA(A/V/E) VVRLTS [SEQ ID NO: 21]
V(A/Q)(E/N/D/K)(A/N)(A/R)(V/Q/I/L)(A/T/N)(A/I)A(Q/E/V/N/H/R/N)AAAEVVR LTX [SEQ ID NO: 22], and/or
wherein the protein further comprises the amino acid sequence: (R/K)(R/K/T)W(S/G)F [SEQ ID NO: 33] or (R/K)(R/K/T)WSF [SEQ ID NO: 34]; preferably wherein this sequence listing is RRWSF [SEQ ID NO: 35] or RKWSF [SEQ ID NO: 36].

50. A method as claimed in claim 49, wherein the protein
(a) further comprises the amino acid sequence: G(Y/D)HP(N/S)YMA(N/C)TES(Y/S)(K/R)(V/A)RS(Q/A)SAP(K/R)(Q/S)R [SEQ ID NO. 23]; and/or
(b) further comprises the amino acid sequence
[SEQ ID NO: 24]
L(H/Y)SATSRSKRSAFTASSIAPSDCT(Q/K)SCC(Y/D)(A/G)DHPS
YMACTESSRAKARSAPKSRPQL(Y/F)YE(Q/R)(S/P)SSKRFG(Y/F)
VD(L/V)PYCGD(S/T)(R/K)SGPQK(V/G)SALHTSFMNKAYPGSGRL
DRLGMPIGYRY.

51. A method as claimed in claim 47, wherein the protein has an amino acid sequence of SEQ ID NO: 1 or an amino acid sequence of at least 30% identity therewith.

52. A method as claimed in claim 47, wherein the protein has an amino acid sequence of SEQ ID NO. 4 or an amino acid sequence of at least 30% identity therewith.

53. A method as claimed in claim 47, wherein the protein has an amino acid sequence of SEQ ID NO: 7 or an amino acid sequence of at least 30% identity therewith.

54. A genetically modified plant cell having
(a) an increased amount of a IQD protein as defined in claim 47 when compared to a corresponding unmodified plant cell or
(b) having at least one polynucleotide sequence encoding an IQD protein as defined in claim 47 in addition to any naturally occurring homolog(s) of said protein(s) in a corresponding unmodified plant cell.

55. A genetically modified plant cell as claimed in claim 54, wherein the increased amount of IQD protein is at least 2-fold; preferably at least 7-fold, compared to the corresponding unmodified plant cell.

56. A genetically modified plant cell as claimed in claim 54, wherein the at least one additional polynucleotide sequence is under the control of a native promoter for the IQD protein.

57. A genetically modified plant cell as claimed in claim 54, wherein the at least one additional polynucleotide sequence is under the control of an heterologous promoter; optionally a constitutive promoter.

58. A genetically modified plant cell as claimed in claim 54, wherein each polynucleotide sequence is inserted into a different genomic locus.

59. A plant comprising or consisting of genetically modified plant cells as claimed in claim 54, wherein at least some of the leaves of the plant are (i) rounder in shape; and/or (ii) thicker; and/or (iii) contain more than chlorophyll per wet or dry weight, compared to a corresponding unmodified plant.

60. A plant as claimed in claim 59, wherein the at least some leaves have a palisade layer comprising more cells and/or more elongated cells compared to a corresponding unmodified plant.

61. A plant as claimed in claim 59, wherein epinastic leaf curling in a corresponding unmodified plant is abolished.

62. A plant as claimed in claim 59, wherein a stem of the plant is thicker compared a corresponding unmodified plant.

63. A plant as claimed in claim 59, wherein transpiration rate is reduced compared to an unmodified plant kept under the same conditions.

64. A plant as claimed in claim 59, wherein wilting is delayed when water is withheld compared to unmodified plant kept under the same conditions; and/or wherein a wilted plant recovers after re-watering compared to an unmodified plant kept under the same conditions which does not recover.

65. A plant as claimed in claim 59, wherein yield of the plant is increased under drought conditions compared to an unmodified plant kept under the same drought conditions, e.g. wherein the yield is total biomass, organ or part biomass, leaf biomass, yield of fruit or yield of seed.

66. A plant comprising or consisting of genetically modified plant cells as claimed in claim 54, wherein at least some of the leaves of the plant have a rate of photosynthesis which is 10% or greater than the photosynthesis rate of a control unmodified plant under the same conditions.


SEQUENCE LISTING (128 sequences)

SEQ ID NO: 1 - protein, 484 aa, Arabidopsis thaliana [sequence omitted]

SEQ ID NO: 2 - DNA, 3817 bp, Arabidopsis thaliana
attttacttcctcagattcacatgaccaaatcatgtaaccattttccaaataaaatctttacatttagatttagattcagaggaattgaattagcctcatcataatgtatgatactacatactacactagtgacacggaaattacacgacgaaaataaacaatgtgaagaataacgaaatttcccgggaaaagagagatagagagtgagacacacgcgcgagtgatgcgtgtggtagtaaatagaacactgtttgatgatactgctgcgactacttaactcttattacaaagctctctttttgttgtctctctcttgctctctctgcaaaactccggcgagaagagaacgttgtcgtttcattcgtatcaaagtcttcatcagcaatgggaaaagcgtcacggtggtttaggagtctattcggagttaagaaacccgacccgggttatccggatctatccgtcgagacgccttctcggtcaacttcctctaatctcaaacgccgatggagtttcgtcaaatccaaacgagaaaaagaaagtacaccgattaatcaagttcctcatactccatcgctaccgaattcgacgcctccaccgccgtctcaccaccaatcgtcgccgagacgacggagaaaacaaaagccgatgtgggaggatgagggaagtgaagattcggacaagcatgctattgcggtggctgccgcgactgctgcggttgctgaagctgcagtcgccgccgctaatgctgctgctgcggtcgtcaggctgacgagcacaagtgggaggtcgactcgaagtcctgttaaggcacggtttagcgacggattcgacgacgtggtggcgcatggtagcaagttttatggacacggccgtgacagttgtgaacttgcggtgattaagatacaatctatatttcgcggatacttggtaattttctttctctaaaatttatttcactcgttttataaattactttatgaagccaaaaatagaaatatacatgaattgcatttttttagtcaaaagtttataatttttgattaaacgaaagctgaatgaatacatttgtgttcatcatttacacgattttgcataattatatttttaattaggattagaaaagtataaaatggttatagatttgttaaacattcacggggattatcgtccagataacatttttcgatgtcgtaaaccttctgctgctaccacttccctagacgagtgataggacaattaaccttccatattctcatttaattattttaatatcttagcagagcacttgaatattctttgtaaagtaatttgctttgtgaataccaaatcaaattgtatttgctttttccttaattttatattcactggttagaaatttgtttttcaatttaaccgactcaaaaattgattccggtccgaacagtatagatgattctcaacaagtttaatgctaagtttaatattatatccattcttccaattaattcgatcctcaacaagtttaatgctaagttgttttcttttttttttttcttttagtttccccatttggttacgagtaaatgaaagatttgtataaaaatacttagtggaaaatcagttaacatcaaccaatacgaagaagtggtcgtactcttgttctattggttagtgaatgttattcactggaccactgtataaataacatggtgaatgctggctgctataattagtctatatttcagttcattctctatatgttatagtctctatgtagtgaatatactatgtctattaatgtttcgatgcatgcctattgaaactttatactttatagcttatggcgaatcttacaaaaaaaaaattttggaggggttttaagataatagtgtgtaagacaattcacacccaaatgtgtactcatcgacataagtcgcattgttcattaatagtcgattcatcttttctctcgtagagagtgactatacatgtaaatgttttttttatatatgtccctctatttaattttgttgaattcctcgtgaatatttcaaaagttcaatttctttaaaaggtcaatatgttttctttctttttccctttcttttggcgccaacttatatcaaaagtaatgctttttcaaatttcaatatgcttttgtggtcctgtccataatgagagcttgagctagtcaagactcttaatgtttttttaagagttgatacttagaatatgatcttgtgattattggctggcttatctccatttctaaggcaatttgttattgtttgattattttaattgtaggcaaagagagcgttaagggcactcaagggtttggttaggcttcaagcgatagttagaggccatatcgaaagaaagagaatgtcagtccatctgcgcaggatgcacgctttggttcgagctcaggctcgtgtgcgtgccactcgggttattgtcacgcctgaatcttcttcttctcaatccaacaataccaaatcttctcacttccaaaaccctgtaagccttgtaaaattccctatgattgttccttttaacctaaaacatgtcttttcgttcaaggtccctttttaacaactcctctgtttttctttaagggtccaccaactccggaaaaactcgagcattcgatctcttctcgcagctccaaactcgctcattctcatcttttcaaggtattacattttcagttgctgtttgtttcttctgtttttgttgcttgtggtcccattagttcaaaatttcagagactatacaagttactgacattgctttatgttcaaaacaaatccaatctcaagaattggtttataggcttctcattgttcctttgttagacttatccatttcacattccatttaaactaagagattggattaaattttcagaggaatggttcgaaggcaagcgacaacaacagactgtaccctgctcacagggagacattctcagccacagacgaagaagaaaagattcttcaaatcgacaggaaacacatcagttcttacacaagacgcaacagaccagacatgttctactcatcccacctcatcctagacaatgctggcctgtctgaaccagttttcgccacgccttttagcccgtcctcgtcgcatgaagagattacaagccagttttgcactgcagagaacagtcctcagttatactcagctacttctagaagcaaacgcagtgctttcaccgctagttctatagcaccgagcgattgcacaaaaagctgctgtgatggtgaccatccaagctacatggcttgtacagagtcctctagggctaaggctaggtccgctagtgccccgaagtctcgaccacagttattttacgagcggccttcatcaaaacggtttggatttgttgatttgccgtactgtggtgatacaaagtccggtccccagaaaggctctgctctgcatactagttttatgaacaaggcttatcccggttcaggtcggttggaccgtctcgggatgccaattgggtataggtactgagaacgttgttggaccctttaacctggttttgttgtagatcagtggattgagctttgcttctaattttctttgtttgtagtttggagctaagtggatttaatctaaaatgcaaatagaacttgcttaatcaaaatgtttgcatctctataaagcattgtcatttgcctagactttagttgttatcaaaaattttatgaatatgaaggaaaccaagagctataacagatatatttggtgtaataacaaaaacgtctaacactaaaaaaattgacaagaaaatgtcatgtccattgggatttagaggag

SEQ ID NO: 3 - DNA, 2146 bp, Arabidopsis thaliana
attttacttcctcagattcacatgaccaaatcatgtaaccattttccaaataaaatctttacatttagatttagattcagaggaattgaattagcctcatcataatgtatgatactacatactacactagtgacacggaaattacacgacgaaaataaacaatgtgaagaataacgaaatttcccgggaaaagagagatagagagtgagacacacgcgcgagtgatgcgtgtggtagtaaatagaacactgtttgatgatactgctgcgactacttaactcttattacaaagctctctttttgttgtctctctcttgctctctctgcaaaactccggcgagaagagaacgttgtcgtttcattcgtatcaaagtcttcatcagcaatgggaaaagcgtcacggtggtttaggagtctattcggagttaagaaacccgacccgggttatccggatctatccgtcgagacgccttctcggtcaacttcctctaatctcaaacgccgatggagtttcgtcaaatccaaacgagaaaaagaaagtacaccgattaatcaagttcctcatactccatcgctaccgaattcgacgcctccaccgccgtctcaccaccaatcgtcgccgagacgacggagaaaacaaaagccgatgtgggaggatgagggaagtgaagattcggacaagcatgctattgcggtggctgccgcgactgctgcggttgctgaagctgcagtcgccgccgctaatgctgctgctgcggtcgtcaggctgacgagcacaagtgggaggtcgactcgaagtcctgttaaggcacggtttagcgacggattcgacgacgtggtggcgcatggtagcaagttttatggacacggccgtgacagttgtgaacttgcggtgattaagatacaatctatatttcgcggatacttggcaaagagagcgttaagggcactcaagggtttggttaggcttcaagcgatagttagaggccatatcgaaagaaagagaatgtcagtccatctgcgcaggatgcacgctttggttcgagctcaggctcgtgtgcgtgccactcgggttattgtcacgcctgaatcttcttcttctcaatccaacaataccaaatcttctcacttccaaaaccctggtccaccaactccggaaaaactcgagcattcgatctcttctcgcagctccaaactcgctcattctcatcttttcaagaggaatggttcgaaggcaagcgacaacaacagactgtaccctgctcacagggagacattctcagccacagacgaagaagaaaagattcttcaaatcgacaggaaacacatcagttcttacacaagacgcaacagaccagacatgttctactcatcccacctcatcctagacaatgctggcctgtctgaaccagttttcgccacgccttttagcccgtcctcgtcgcatgaagagattacaagccagttttgcactgcagagaacagtcctcagttatactcagctacttctagaagcaaacgcagtgctttcaccgctagttctatagcaccgagcgattgcacaaaaagctgctgtgatggtgaccatccaagctacatggcttgtacagagtcctctagggctaaggctaggtccgctagtgccccgaagtctcgaccacagttattttacgagcggccttcatcaaaacggtttggatttgttgatttgccgtactgtggtgatacaaagtccggtccccagaaaggctctgctctgcatactagttttatgaacaaggcttatcccggttcaggtcggttggaccgtctcgggatgccaattgggtataggtactgagaacgttgttggaccctttaacctggttttgttgtagatcagtggattgagctttgcttctaattttctttgtttgtagtttggagctaagtggatttaatctaaaatgcaaatagaacttgcttaatcaaaatgtttgcatctctataaagcattgtcatttgcctagactttagttgttatcaaaaattttatgaatatgaaggaaaccaagagctataacagatatatttggtgtaataacaaaaacgtctaacactaaaaaaattgacaagaaaatgtcatgtccattgggatttagaggag

SEQ ID NO: 4 - protein, 403 aa, Arabidopsis thaliana
MGFFGRLFGSKKKSDKAASSRDKRRWSFTTRSSNSSKRAPAVTSASVVEQNGLDADKHAIAVAAATAAVAEAALTAAHAAAEVVRLTSGNGGRNVGGGGNSSVFQIGRSNRRWAQENIAAMKIQSAFRGYLARRALRALKALVKLQALVRGHIVRKQTADMLRRMQTLVRLQSQARARASRSSHSSASFHSSTALLFPSSSSSPRSLHTRCVSNAEVSSLDHRGGSKRLDWQAEESENGDKILEVDTWKPHYHPKPLRSERNNESPRKRQQSLLGPRSTENSPQVGSSGSRRRTPFTPTSRSEYSWGCNNYYYSGYHPNYMANTESYKAKVRSQSAPKQRVEVSNETSGYKRSVQGQYYYYTAVEEESLDVGSAGYYGGGGGDSDRLNRNQSAKSRMHSSFLV

SEQ ID NO: 5 - DNA, 2828 bp, Arabidopsis thaliana
aaaataaaaaagccttttaaaatctcgtgtaactaactcgctgtatgccggcgcgtggaaacgtactctgcaatctatatataagccgttaaactctatttagcttctctatagacaaaagtttatgttttgtttttcctctaacacaaaaaacaatctcagatattaaataaagtgttaatcaaccggagaactctccgacaaaacctcgccgacgattttccgacgatgaaagatgggctttttcgggagactgttcgggagtaaaaaaaagtctgataaagctgcttcgtcgagagataaacggaggtggagcttcaccaccagatcttcaaattccagcaagagagctccggcggtgacgtcggcttcggtggttgagcaaaatggtttggatgctgacaaacatgcgatagctgtggctgctgcgacagccgcggtggcggaagcagctcttactgctgctcatgcggcagctgaagtcgtgagactaacaagcggaaacggcgggagaaacgtcggtggcggtggaaactcgtcggtgtttcaaataggaagaagtaaccgtcggtgggctcaggagaatatcgcggcgatgaagattcaatccgcttttcgtggttatctggttagtttactaatttctcaaacgttttagctgattaattcaagaatgattcaaaatgttgaattttgaaactgttcgaaaatttcataaaacaatttacataatgtgcaataactgttgaaattcggattttttttcttctcattcacaatttcaaattttgaaaatggaaacaatgggtaaaacttgtattgttttgtgtggggtttggtcccaaaagaaactgactatagaagaagaagaagaagacaaacaaatgtgaaatgaagaaggttctttgaaacttgtacactctgccgacactatttttagtggttatcaatagttccagtttttttttttatcttcttctgttagtttatcactcaaaattttgatgaaagatctgatcagaatgtcgaaatgaacttagtttaatgttggtgtgaatcatttgattttagttgtcagatgggccaagtttttttttgttccacgttatgttgtgctttataattctgaacctttattgaaattaacaagactaattagtattttacagatgggttcttttttgctttaggacagcaactttgaagttgaaatctttagcccaaaaactttttcaagttgcttatttatcttctctttacttacaaagattcattttgaccatgttttttactctttttcaaattcctatctgtgaatcttggagttttttttgtttttgctaatttgcgttaaattattccaagatcttatgtgtgttgtcgaatcttgaccaatgtgtaaagaagaactcgtgtgtgtttgtgtgtgtacatttgactttgagacatagttatgagaatggtgggctctgttgtaattactaaatctagtttttggtacaagtggaagtggttgagaaactaaaagctaaatagaataatttcacaaacaactaatccctttactactctttttctgtgtgttttaatgttagttggcaaaacgatgattatgtcttcaacaaatgattgaactcgccttgtctttgttattcactttcaagcaaactcaaattttgattattcatggtggagccaattttttactccaagttctttctatatggagtaaatattcattttttgaacataaattaaagactctatatgaagtttattggtttgctttggctactttggaaaattttcaggcgaggagagcattacgagcactaaaggcattagtgaagcttcaagcattagtgagaggacacatagtgagaaaacaaactgcagatatgcttagaaggatgcagactcttgttcgtcttcaatctcaagctcgtgctcgagcctctcgttcttctcactcctctgcttctttccactcctccaccgctcttttgttcccatcttcctcgtcttctccacgttctcttcacacgcgctgcgtttcaaacgctgaagtcagctctcttgaccaccgtggaggctctaagcggttagattggcaggcggaggaaagcgaaaatggagacaagatcctagaagtggatacttggaagcctcattatcatcccaaaccgttacgttcagagagaaacaatgagtctccgaggaaacgacaacaatctttgttgggtccgagaagtacagagaatagtcctcaagttggttctagtgggtcaagaagaagaactccttttacgccgacgtcaagaagcgagtattcttggggatgtaataactattactactcgggttatcacccgaattacatggctaacactgagtcttataaagctaaagttcggtcacaaagtgcgccgaaacagagagttgaggtctctaatgagaccagtggctacaagagatctgttcagggacagtattactactacacagcggtagaagaagagagtttggatgttggaagcgctggttactacggaggaggaggaggcgattctgatcgattgaatcggaaccaaagtgcgaaatcgaggatgcattcttcgtttcttgtttagattgtgattctctttctcttcttttttttggtttgagtttggtaattttccagaagagataagtattaacactggatgtgtaaattgttgttaacaagtttcgattgcttgcaaaagaaggaacaccactctgttctctatctttgttgattagtgttcaattaagattaatcaaactgagtcaaaccccacaattta

SEQ ID NO: 6 - DNA, 1645 bp, Arabidopsis thaliana
aaaataaaaaagccttttaaaatctcgtgtaactaactcgctgtatgccggcgcgtggaaacgtactctgcaatctatatataagccgttaaactctatttagcttctctatagacaaaagtttatgttttgtttttcctctaacacaaaaaacaatctcagatattaaataaagtgttaatcaaccggagaactctccgacaaaacctcgccgacgattttccgacgatgaaagatgggctttttcgggagactgttcgggagtaaaaaaaagtctgataaagctgcttcgtcgagagataaacggaggtggagcttcaccaccagatcttcaaattccagcaagagagctccggcggtgacgtcggcttcggtggttgagcaaaatggtttggatgctgacaaacatgcgatagctgtggctgctgcgacagccgcggtggcggaagcagctcttactgctgctcatgcggcagctgaagtcgtgagactaacaagcggaaacggcgggagaaacgtcggtggcggtggaaactcgtcggtgtttcaaataggaagaagtaaccgtcggtgggctcaggagaatatcgcggcgatgaagattcaatccgcttttcgtggttatctggcgaggagagcattacgagcactaaaggcattagtgaagcttcaagcattagtgagaggacacatagtgagaaaacaaactgcagatatgcttagaaggatgcagactcttgttcgtcttcaatctcaagctcgtgctcgagcctctcgttcttctcactcctctgcttctttccactcctccaccgctcttttgttcccatcttcctcgtcttctccacgttctcttcacacgcgctgcgtttcaaacgctgaagtcagctctcttgaccaccgtggaggctctaagcggttagattggcaggcggaggaaagcgaaaatggagacaagatcctagaagtggatacttggaagcctcattatcatcccaaaccgttacgttcagagagaaacaatgagtctccgaggaaacgacaacaatctttgttgggtccgagaagtacagagaatagtcctcaagttggttctagtgggtcaagaagaagaactccttttacgccgacgtcaagaagcgagtattcttggggatgtaataactattactactcgggttatcacccgaattacatggctaacactgagtcttataaagctaaagttcggtcacaaagtgcgccgaaacagagagttgaggtctctaatgagaccagtggctacaagagatctgttcagggacagtattactactacacagcggtagaagaagagagtttggatgttggaagcgctggttactacggaggaggaggaggcgattctgatcgattgaatcggaaccaaagtgcgaaatcgaggatgcattcttcgtttcttgtttagattgtgattctctttctcttcttttttttggtttgagtttggtaattttccagaagagataagtattaacactggatgtgtaaattgttgttaacaagtttcgattgcttgcaaaagaaggaacaccactctgttctctatctttgttgattagtgttcaattaagattaatcaaactgagtcaaaccccacaattta

SEQ ID NO: 7 - protein, 401 aa, Arabidopsis thaliana
MGFFGRLFGSKKQEKATPNRRRWSFATRSSHPENDSSSHSSKRRGDEDVLNGDKHAIAVAAATAAVAEAALAAARAAAEVVRLTNGGRNSSVKQISRSNRRWSQEYKAAMKIQSAFRGYLARRALRALKALVKLQALVKGHIVRKQTADMLRRMQTLVRLQARARASRSSHVSDSSHPPTLMIPSSPQSFHARCVSEAEYSKVIAMDHHHNNHRSPMGSSRLLDQWRTEESLWSAPKYNEDDDKILEVDTWKPHFRESPRKRGSLVVPTSVENSPQLRSRTGSSSGGSRRKTPFTPARSEYEYYSGYHPNYMANTESYKAKVRSQSAPRQRLQDLPSESGYKRSIQGQYYYYTPAAERSFDQRSDNGIAGYRGVSDGLDRNQSDKSKMYTSFFSSNPLFFQ

SEQ ID NO: 8 - DNA, 2382 bp, Arabidopsis thaliana
ttctccattcccaattgtctcttcttttctttttgtacttgtcaaaaacaaaaagaacaacaaaaaaaatctcaaccgtagaaaattccgacaagagttcagttcatacaatgaactaagtatgggtttctttggaagactgttcggaagtaagaagcaagaaaaggcaacaccgaacagacgaagatggagcttcgctactagatcctcacatcccgagaatgattcgtcttctcattcaagcaagagacgtggggatgaagatgtcttaaacggcgacaagcatgcgatagccgtcgcggctgctacagctgcagtggctgaagccgcactcgctgctgctcgtgcggcggcggaagtcgtgagactcaccaatggtggtagaaactcgtcggtaaaacaaatcagtcggagtaatcgtcggtggtctcaagagtataaagcagctatgaagattcaatccgcttttcgtggctacttggtgagttaattacttctctaagtttaatcttaatactcaacttcataatctataattttgtttgaaagaaattaagtttttgagccaaagtgtggaatagctgtctgatgttgatttttctacttaaacccaaattgaaatatttactaaacaaaaaaaagaaagaatgagaataatttgtatttatttgtattggtccccgaaaaagactaggaaaggagacaaatgaatgaataatgataccaagaaacttgtttagagccgatacgattctctaccgagtaccgacactatttaatggtactactctagaattgtatagataaaatttggtcccaaaaaatatgcaaatattatctcaaacaaaatcttacctaactagatttagctaagggcttattctttgtaataaattaatttttacattaaaccaagtcagcaataaagaataacaaggagagggccaagttctttattttatctactttctttttagtttccaaagttccaaagtgtattttactataaaggtgaaactacagattggtttttaagctttttgagctttaaagttgcatcaccactgttactatcaaagttgcaacctacctttttcataatttgacagataggttatgtagtttagacacataatttagagattctctaatcttattgcttcgtatataacaaataaggtgtgtatcatttagaagaaaatgatagtggttgggaaactatgagtaatttaataaggagtattagtgtattactttgtggtttgaattaagtgtgtaaccaagttttctttaattcaggcgaggagggcgttgagagcactgaaggcattagtgaagcttcaagcgttggtgaagggacacatagtaaggaaacaaacggctgatatgctgcgtcgaatgcaaacgctggttcggctccaagcacgagctagagcttcgcgttcttctcacgtttctgactcttcccatccgccaacactaatgattccatcttccccacaatctttccatgcacgatgcgtttcagaggctgagtacagtaaagtcattgccatggatcaccaccacaacaaccaccgttcaccgatgggttcaagccggttattagaccaatggaggacagaggaaagtctatggagcgcaccaaagtacaatgaagatgatgacaaaatcctagaagtcgacacttggaagcctcacttcagagagtcaccaaggaaaagaggatctctagtggttcctacaagtgtggagaacagtccacaattaaggtctagaacaggaagcagcagtggtggttcaaggagaaaaactcccttcacgcctgcgagaagcgagtacgagtactactctgggtatcaccctaactacatggctaacactgagtcttacaaagcaaaagtccgatcacaaagcgcaccaagacagagactacaagatttaccttcagagagtggttacaagaggtctatacagggacagtattactactacacgcctgctgcagagcgatcgtttgatcagcgttcggataacgggatcgcgggttacagaggagtttctgatgggttagatcgaaaccaaagtgacaaatcgaagatgtacacttcgtttttcagttctaatcctcttttctttcaatagtcgagaaaggatgaaaaaagtgagtggaatgtgtaaaattagatttcgacacacgagtacagagacagccagtgatcaatctgtgttttgtactattttctaattgactgtatccaacaagggtccattcttgtctgataaaaaaacttcaataatttgaagtgatgtcaagtcaagacgtgggaatcaccacttaaagcaatgaaaattgattgatataacctttcatattaaact

SEQ ID NO: 9 - DNA, 1564 bp, Arabidopsis thaliana
ttctccattcccaattgtctcttcttttctttttgtacttgtcaaaaacaaaaagaacaacaaaaaaaatctcaaccgtagaaaattccgacaagagttcagttcatacaatgaactaagtatgggtttctttggaagactgttcggaagtaagaagcaagaaaaggcaacaccgaacagacgaagatggagcttcgctactagatcctcacatcccgagaatgattcgtcttctcattcaagcaagagacgtggggatgaagatgtcttaaacggcgacaagcatgcgatagccgtcgcggctgctacagctgcagtggctgaagccgcactcgctgctgctcgtgcggcggcggaagtcgtgagactcaccaatggtggtagaaactcgtcggtaaaacaaatcagtcggagtaatcgtcggtggtctcaagagtataaagcagctatgaagattcaatccgcttttcgtggctacttggcgaggagggcgttgagagcactgaaggcattagtgaagcttcaagcgttggtgaagggacacatagtaaggaaacaaacggctgatatgctgcgtcgaatgcaaacgctggttcggctccaagcacgagctagagcttcgcgttcttctcacgtttctgactcttcccatccgccaacactaatgattccatcttccccacaatctttccatgcacgatgcgtttcagaggctgagtacagtaaagtcattgccatggatcaccaccacaacaaccaccgttcaccgatgggttcaagccggttattagaccaatggaggacagaggaaagtctatggagcgcaccaaagtacaatgaagatgatgacaaaatcctagaagtcgacacttggaagcctcacttcagagagtcaccaaggaaaagaggatctctagtggttcctacaagtgtggagaacagtccacaattaaggtctagaacaggaagcagcagtggtggttcaaggagaaaaactcccttcacgcctgcgagaagcgagtacgagtactactctgggtatcaccctaactacatggctaacactgagtcttacaaagcaaaagtccgatcacaaagcgcaccaagacagagactacaagatttaccttcagagagtggttacaagaggtctatacagggacagtattactactacacgcctgctgcagagcgatcgtttgatcagcgttcggataacgggatcgcgggttacagaggagtttctgatgggttagatcgaaaccaaagtgacaaatcgaagatgtacacttcgtttttcagttctaatcctcttttctttcaatagtcgagaaaggatgaaaaaagtgagtggaatgtgtaaaattagatttcgacacacgagtacagagacagccagtgatcaatctgtgttttgtactattttctaattgactgtatccaacaagggtccattcttgtctgataaaaaaacttcaataatttgaagtgatgtcaagtcaagacgtgggaatcaccacttaaagcaatgaaaattgattgatataacctttcatattaaact

SEQ ID NO: 10 - protein, 25 aa, Arabidopsis thaliana
FRGYLARRALRALKGLVRLQALVRG

SEQ ID NO: 11 - protein, 67 aa, Artificial Sequence
  IQ67 Domain Consensus
  MISC_FEATURE (3)..(3): X is any hydrophobic amino acid
  MISC_FEATURE (6)..(6): X is any hydrophobic amino acid
  MISC_FEATURE (7)..(7): X is any basic amino acid
  MISC_FEATURE (10)..(10): X is any amino acid
  MISC_FEATURE (11)..(11): X is any hydrophobic amino acid
  MISC_FEATURE (37)..(37): X is any amino acid
  MISC_FEATURE (38)..(38): X is any hydrophobic amino acid
  MISC_FEATURE (41)..(41): X is any basic amino acid
  MISC_FEATURE (44)..(45): X is any hydrophobic amino acid
  MISC_FEATURE (48)..(48): X is any basic amino acid
  MISC_FEATURE (56)..(56): X is any hydrophobic amino acid
  MISC_FEATURE (59)..(59): X is any amino acid
  MISC_FEATURE (65)..(65): X is any hydrophobic amino acid
  MISC_FEATURE (66)..(66): X is any basic amino acid
  MISC_FEATURE (67)..(67): X is any hydrophobic amino acid
EEXAAXXIQXXFRGYLARRALRALKGLVRLQALVRGXXVRXQAXXTLXCMQALVRXQAXVRARRXXX

SEQ ID NO: 12 - DNA, 1455 bp, Artificial Sequence
  AT4G23060.1 IQD22
atgggaaaagcgtcacggtggtttaggagtctattcggagttaagaaacccgacccgggttatccggatctatccgtcgagacgccttctcggtcaacttcctctaatctcaaacgccgatggagtttcgtcaaatccaaacgagaaaaagaaagtacaccgattaatcaagttcctcatactccatcgctaccgaattcgacgcctccaccgccgtctcaccaccaatcgtcgccgagacgacggagaaaacaaaagccgatgtgggaggatgagggaagtgaagattcggacaagcatgctattgcggtggctgccgcgactgctgcggttgctgaagctgcagtcgccgccgctaatgctgctgctgcggtcgtcaggctgacgagcacaagtgggaggtcgactcgaagtcctgttaaggcacggtttagcgacggattcgacgacgtggtggcgcatggtagcaagttttatggacacggccgtgacagttgtgaacttgcggtgattaagatacaatctatatttcgcggatacttggcaaagagagcgttaagggcactcaagggtttggttaggcttcaagcgatagttagaggccatatcgaaagaaagagaatgtcagtccatctgcgcaggatgcacgctttggttcgagctcaggctcgtgtgcgtgccactcgggttattgtcacgcctgaatcttcttcttctcaatccaacaataccaaatcttctcacttccaaaaccctggtccaccaactccggaaaaactcgagcattcgatctcttctcgcagctccaaactcgctcattctcatcttttcaagaggaatggttcgaaggcaagcgacaacaacagactgtaccctgctcacagggagacattctcagccacagacgaagaagaaaagattcttcaaatcgacaggaaacacatcagttcttacacaagacgcaacagaccagacatgttctactcatcccacctcatcctagacaatgctggcctgtctgaaccagttttcgccacgccttttagcccgtcctcgtcgcatgaagagattacaagccagttttgcactgcagagaacagtcctcagttatactcagctacttctagaagcaaacgcagtgctttcaccgctagttctatagcaccgagcgattgcacaaaaagctgctgtgatggtgaccatccaagctacatggcttgtacagagtcctctagggctaaggctaggtccgctagtgccccgaagtctcgaccacagttattttacgagcggccttcatcaaaacggtttggatttgttgatttgccgtactgtggtgatacaaagtccggtccccagaaaggctctgctctgcatactagttttatgaacaaggcttatcccggttcaggtcggttggaccgtctcgggatgccaattgggtataggtactga

SEQ ID NO: 13 - DNA, 1212 bp, Artificial Sequence
  >AT5G62070.1  IQD23
atgggctttttcgggagactgttcgggagtaaaaaaaagtctgataaagctgcttcgtcgagagataaacggaggtggagcttcaccaccagatcttcaaattccagcaagagagctccggcggtgacgtcggcttcggtggttgagcaaaatggtttggatgctgacaaacatgcgatagctgtggctgctgcgacagccgcggtggcggaagcagctcttactgctgctcatgcggcagctgaagtcgtgagactaacaagcggaaacggcgggagaaacgtcggtggcggtggaaactcgtcggtgtttcaaataggaagaagtaaccgtcggtgggctcaggagaatatcgcggcgatgaagattcaatccgcttttcgtggttatctggcgaggagagcattacgagcactaaaggcattagtgaagcttcaagcattagtgagaggacacatagtgagaaaacaaactgcagatatgcttagaaggatgcagactcttgttcgtcttcaatctcaagctcgtgctcgagcctctcgttcttctcactcctctgcttctttccactcctccaccgctcttttgttcccatcttcctcgtcttctccacgttctcttcacacgcgctgcgtttcaaacgctgaagtcagctctcttgaccaccgtggaggctctaagcggttagattggcaggcggaggaaagcgaaaatggagacaagatcctagaagtggatacttggaagcctcattatcatcccaaaccgttacgttcagagagaaacaatgagtctccgaggaaacgacaacaatctttgttgggtccgagaagtacagagaatagtcctcaagttggttctagtgggtcaagaagaagaactccttttacgccgacgtcaagaagcgagtattcttggggatgtaataactattactactcgggttatcacccgaattacatggctaacactgagtcttataaagctaaagttcggtcacaaagtgcgccgaaacagagagttgaggtctctaatgagaccagtggctacaagagatctgttcagggacagtattactactacacagcggtagaagaagagagtttggatgttggaagcgctggttactacggaggaggaggaggcgattctgatcgattgaatcggaaccaaagtgcgaaatcgaggatgcattcttcgtttcttgtttag

SEQ ID NO: 14 - DNA, 1344 bp, Artificial Sequence
  AT5G07240.2  IQD24
atgtatcacactttagcttctccattcccaattgtctcttcttttctttttgtacttgtcaaaaacaaaaagaacaacaaaaaaaatctcaaccgtagaaaattccgacaagagttcagttcatacaatgaactaagtatgggtttctttggaagactgttcggaagtaagaagcaagaaaaggcaacaccgaacagacgaagatggagcttcgctactagatcctcacatcccgagaatgattcgtcttctcattcaagcaagagacgtggggatgaagatgtcttaaacggcgacaagcatgcgatagccgtcgcggctgctacagctgcagtggctgaagccgcactcgctgctgctcgtgcggcggcggaagtcgtgagactcaccaatggtggtagaaactcgtcggtaaaacaaatcagtcggagtaatcgtcggtggtctcaagagtataaagcagctatgaagattcaatccgcttttcgtggctacttggcgaggagggcgttgagagcactgaaggcattagtgaagcttcaagcgttggtgaagggacacatagtaaggaaacaaacggctgatatgctgcgtcgaatgcaaacgctggttcggctccaagcacgagctagagcttcgcgttcttctcacgtttctgactcttcccatccgccaacactaatgattccatcttccccacaatctttccatgcacgatgcgtttcagaggctgagtacagtaaagtcattgccatggatcaccaccacaacaaccaccgttcaccgatgggttcaagccggttattagaccaatggaggacagaggaaagtctatggagcgcaccaaagtacaatgaagatgatgacaaaatcctagaagtcgacacttggaagcctcacttcagagagtcaccaaggaaaagaggatctctagtggttcctacaagtgtggagaacagtccacaattaaggtctagaacaggaagcagcagtggtggttcaaggagaaaaactcccttcacgcctgcgagaagcgagtacgagtactactctgggtatcaccctaactacatggctaacactgagtcttacaaagcaaaagtccgatcacaaagcgcaccaagacagagactacaagatttaccttcagagagtggttacaagaggtctatacagggacagtattactactacacgcctgctgcagagcgatcgtttgatcagcgttcggataacgggatcgcgggttacagaggagtttctgatgggttagatcgaaaccaaagtgacaaatcgaagatgtacacttcgtttttcagttctaatcctcttttctttcaatag

SEQ ID NO: 15 - protein, 16 aa, Artificial Sequence
  peptide
HAIAVAAATAAVAEAA

SEQ ID NO: 16 - protein, 14 aa, Artificial Sequence
  peptide
  MISC_FEATURE (1)..(1): X is one of the amino acids D, R, S, N
  MISC_FEATURE (2)..(2): X is one of the amino acids K, Q, D, N, R, T
  MISC_FEATURE (3)..(3): X is one of the amino acids H, R
  MISC_FEATURE (4)..(4): X is one of the amino acids A, S
  MISC_FEATURE (5)..(5): X is one of the amino acids I, V, M, S
  MISC_FEATURE (6)..(6): X is one of the amino acids A, H
  MISC_FEATURE (8)..(8): X is one of the amino acids A, T
  MISC_FEATURE (9)..(9): X is one of the amino acids A, E, F
  MISC_FEATURE (10)..(10): X is one of the amino acids A, P
  MISC_FEATURE (12)..(12): X is one of the amino acids A, E
  MISC_FEATURE (13)..(13): X is one of the amino acids A, H, V
  MISC_FEATURE (14)..(14): X is one of the amino acids V, A
XXXXXXVXXXTXXX

SEQ ID NO: 17 - protein, 14 aa, Artificial Sequence
  peptide
DKHAIAVAAATAAV

SEQ ID NO: 18 - protein, 14 aa, Artificial Sequence
  peptide
DKHAIAVAAATAAV

SEQ ID NO: 19 - protein, 20 aa, Artificial Sequence
  peptide
  MISC_FEATURE (2)..(2): X is one of the amino acids A, Q
  MISC_FEATURE (3)..(3): X is one of the amino acids E, N, D, K
  MISC_FEATURE (4)..(4): X is one of the amino acids A, N
  MISC_FEATURE (5)..(5): X is one of the amino acids A, R
  MISC_FEATURE (6)..(6): X is one of the amino acids V, Q, I, L
  MISC_FEATURE (7)..(7): X is one of the amino acids A, T, N
  MISC_FEATURE (8)..(8): X is one of the amino acids A, I
  MISC_FEATURE (10)..(10): X is one of the amino acids Q, E, V, N, H, R, N
  MISC_FEATURE (14)..(14): X is one of the amino acids A, V, E
  MISC_FEATURE (17)..(17): X is one of the amino acids R, K, E, S, L, T
  MISC_FEATURE (18)..(18): X is one of the amino acids L, F
  MISC_FEATURE (19)..(19): X is one of the amino acids T, N
  MISC_FEATURE (20)..(20): X is any amino acids
VXXXXXXXAXAAAXVVXXXX

SEQ ID NO: 20 - protein, 20 aa, Artificial Sequence
  peptide
  MISC_FEATURE (2)..(2): X is any of amino acids A, Q
  MISC_FEATURE (3)..(3): X is any of amino acids E, N, D, K
  MISC_FEATURE (4)..(4): X is any of amino acids A, N
  MISC_FEATURE (5)..(5): X is any of amino acids A, R
  MISC_FEATURE (6)..(6): X is any of amino acids V, Q, I, L
  MISC_FEATURE (7)..(7): X is any of amino acids A, T, N
  MISC_FEATURE (8)..(8): X is any of amino acids A, I
  MISC_FEATURE (10)..(10): X is any of amino acids Q, E, V, N, H, R, N
  MISC_FEATURE (14)..(14): X is any of amino acids A, V, E
  MISC_FEATURE (20)..(20): X is any amino acid
VXXXXXXXAXAAAXVVRLTX

SEQ ID NO: 21 - protein, 20 aa, Artificial Sequence
  peptide
  MISC_FEATURE (2)..(2): X is any one of amino acids A, Q
  MISC_FEATURE (3)..(3): X is any one of amino acids E, N, D, K
  MISC_FEATURE (4)..(4): X is any one of amino acids A, N
  MISC_FEATURE (5)..(5): X is any one of amino acids A, R
  MISC_FEATURE (6)..(6): X is any one of amino acids V, Q, I, L
  MISC_FEATURE (7)..(7): X is any one of amino acids A, T, N
  MISC_FEATURE (8)..(8): X is any one of amino acids A, I
  MISC_FEATURE (10)..(10): X is any one of amino acids Q, E, V, N, H, R, N
  MISC_FEATURE (14)..(14): X is any one of amino acids A, V, E
VXXXXXXXAXAAAXVVRLTS

SEQ ID NO: 22 - protein, 20 aa, Artificial Sequence
  peptide
  MISC_FEATURE (2)..(2): X is any one of amino acids A, Q
  MISC_FEATURE (3)..(3): X is any one of amino acids E, N, D, K
  MISC_FEATURE (4)..(4): X is any one of amino acids A, N
  MISC_FEATURE (5)..(5): X is any one of amino acids A, R
  MISC_FEATURE (6)..(6): X is any one of amino acids V, Q, I, L
  MISC_FEATURE (7)..(7): X is any one of amino acids A, T, N
  MISC_FEATURE (8)..(8): X is any one of amino acids A, I
  MISC_FEATURE (10)..(10): X is any one of amino acids Q, E, V, N, H, R, N
  MISC_FEATURE (20)..(20): X is any amino acid
VXXXXXXXAXAAAEVVRLTX

SEQ ID NO: 23 - protein, 24 aa, Artificial Sequence
  peptide
  MISC_FEATURE (2)..(2): X is any of amino acids Y, D
  MISC_FEATURE (5)..(5): X is any of amino acids N, S
  MISC_FEATURE (9)..(9): X is any of amino acids N, C
  MISC_FEATURE (13)..(13): X is any of amino acids Y, S
  MISC_FEATURE (14)..(14): X is any of amino acids K, R
  MISC_FEATURE (15)..(15): X is any of amino acids V, A
  MISC_FEATURE (18)..(18): X is any of amino acids Q, A
  MISC_FEATURE (22)..(22): X is any of amino acids K, R
  MISC_FEATURE (23)..(23): X is any of amino acids Q, S
GXHPXYMAXTESXXXRSXSAPXXR

SEQ ID NO: 24 - protein, 113 aa, Artificial Sequence
  C-terminal portion
  MISC_FEATURE (2)..(2): X is any one of amino acids H, Y
  MISC_FEATURE (25)..(25): X is any one of amino acids Q, K
  MISC_FEATURE (29)..(29): X is any one of amino acids Y, D
  MISC_FEATURE (30)..(30): X is any one of amino acids A, G
  MISC_FEATURE (57)..(57): X is any one of amino acids Y, F
  MISC_FEATURE (60)..(60): X is any one of amino acids Q, R
  MISC_FEATURE (61)..(61): X is any one of amino acids S, P
  MISC_FEATURE (68)..(68): X is any one of amino acids Y, F
  MISC_FEATURE (71)..(71): X is any one of amino acids L, V
  MISC_FEATURE (77)..(77): X is any one of amino acids S, T
  MISC_FEATURE (78)..(78): X is any one of amino acids R, K
  MISC_FEATURE (84)..(84): X is any one of amino acids V, G
LXSATSRSKRSAFTASSIAPSDCTXSCCXXDHPSYMACTESSRAKARSAPKSRPQLXYEXXSSKRFGXVDXPYCGDXXSGPQKXSALHTSFMNKAYPGSGRLDRLGMPIGYRY

SEQ ID NO: 25 - protein, 6 aa, Artificial Sequence
  peptide
  MISC_FEATURE (1)..(1): X is any amino acid or K
XRRWSF

SEQ ID NO: 26 - protein, 18 aa, Artificial Sequence
  peptide
DKHAIAVAAATAAVAEAA

SEQ ID NO: 27 - protein, 6 aa, Artificial Sequence
  peptide
  MISC_FEATURE (6)..(6): X is any amino acid or S
VVRLTX

SEQ ID NO: 28 - protein, 4 aa, Artificial Sequence
  peptide
KIQS

SEQ ID NO: 29 - protein, 6 aa, Artificial Sequence
  peptide
FRGYLA

SEQ ID NO: 30 - protein, 7 aa, Artificial Sequence
  peptide
RALRALK

SEQ ID NO: 31 - protein, 6 aa, Artificial Sequence
  peptide
  MISC_FEATURE (6)..(6): X is any amino acid or L
ENSPQX

SEQ ID NO: 32 - protein, 5 aa, Artificial Sequence
  peptide
VRGHI

SEQ ID NO: 33 - protein, 5 aa, Artificial Sequence
  peptide
  MISC_FEATURE (1)..(1): x is any one of amino acids R, K
  MISC_FEATURE (2)..(2): x is any one of amino acids R, K, T
  MISC_FEATURE (4)..(4): x is any one of amino acids S, G
XXWXF

SEQ ID NO: 34 - protein, 5 aa, Artificial Sequence
  peptide
  MISC_FEATURE (1)..(1): X is anyone of amino acids R, K
  MISC_FEATURE (2)..(2): X is anyone of amino acids R, K, T
XXWSF

SEQ ID NO: 35 - protein, 5 aa, Artificial Sequence
  peptide
RRWSF

SEQ ID NO: 36 - protein, 5 aa, Artificial Sequence
  peptide
RKWSF

SEQ ID NO: 37 - protein, 19 aa, Artificial Sequence
  peptide
  MISC_FEATURE (19)..(19): X is any amino acid, or L
DKHAIAVAAATAAVAEAAX

SEQ ID NO: 38 - protein, 12 aa, Artificial Sequence
  peptide
MGFFGRLFGSKK

SEQ ID NO: 39 - protein, 9 aa, Artificial Sequence
  peptide
AAAEVVRLT

SEQ ID NO: 40 - protein, 6 aa, Artificial Sequence
  peptide
RSNRRW

SEQ ID NO: 41 - protein, 31 aa, Artificial Sequence
  peptide
AAMKIQSAFRGYLARRALRALKALVKLQALV

SEQ ID NO: 42 - protein, 22 aa, Artificial Sequence
  peptide
GHIVRKQTADMLRRMQTLVRLQ

SEQ ID NO: 43 - protein, 10 aa, Artificial Sequence
  peptide
ARARASRSSH

SEQ ID NO: 44 - protein, 12 aa, Artificial Sequence
  peptide
DKILEVDTWKPH

SEQ ID NO: 45 - protein, 6 aa, Artificial Sequence
  peptide
ESPRKR

SEQ ID NO: 46 - protein, 5 aa, Artificial Sequence
  peptide
TPFTP

SEQ ID NO: 47 - protein, 26 aa, Artificial Sequence
  peptide
YYSGYHPNYMANTESYKAKVRSQSAP

SEQ ID NO: 48 - protein, 6 aa, Artificial Sequence
  peptide
SGYKRS

SEQ ID NO: 49 - protein, 8 aa, Artificial Sequence
  peptide
QGQYYYYT

SEQ ID NO: 50 - protein, 9 aa, Artificial Sequence
  peptide
  MISC_FEATURE (1)..(4): X is any amino acid
XXXXVVRLT

SEQ ID NO: 51 - protein, 8 aa, Artificial Sequence
  peptide
  MISC_FEATURE (7)..(8): X is any amino acid
RSNRRWXX

SEQ ID NO: 52 - protein, 8 aa, Artificial Sequence
  peptide
  MISC_FEATURE (1)..(3): X is any amino acid
XXXRRWSF

SEQ ID NO: 53 - protein, 6 aa, Artificial Sequence
  peptide
ENSPQL

SEQ ID NO: 54 - protein, 5 aa, Artificial Sequence
  peptide
ENSPQ

SEQ ID NO: 55 - protein, 5 aa, Artificial Sequence
  peptide
VVRLT

SEQ ID NO: 56 - protein, 19 aa, Artificial Sequence
  peptide
DKHAIAVAAATAAVAEAAL

SEQ ID NO: 57 - DNA, 25 bp, Artificial Sequence
  Primer Ncol F
ccatgggaaaagcgtcacggtggtt

SEQ ID NO: 58 - DNA, 30 bp, Artificial Sequence
  Primer EcoRV R
gatatctcagtacctatacccaattggcat

SEQ ID NO: 59 - protein, 30 aa, Gossypium hirsutum
RSDKERRRWSFARSSNPTSSTSHIDHALTT

SEQ ID NO: 60 - protein, 35 aa, Gossypium arborense
LFGAKKSDKTRSDKERRRWSFARSSNPTSSTSHID

SEQ ID NO: 61 - protein, 32 aa, Theobroma cacao
KKSAQARPDRDRRRWSFARSSNPTSSSSHRDA

SEQ ID NO: 62 - protein, 39 aa, Artificial Sequence
  Bol043865 IQD24
KKKQEKSSLTNNNRRKWSFTARSSNPATALSASSSHPSK

SEQ ID NO: 63 - protein, 35 aa, Artificial Sequence
  Bol043865 IQD23
EKSSLTNNNRRKWSFTARSSNPATALSASSSHPSK

SEQ ID NO: 64 - protein, 38 aa, Artificial Sequence
  Bra009281 IQD24
KKKQEKSSLTNNNRRKWSFTTRSSNPATALSASSSHPS

SEQ ID NO: 65 - protein, 36 aa, Eutrema salsugineum
SKKQEKPAPTKNNRRRWSFTTRSSNQATASSAASST

SEQ ID NO: 66 - protein, 27 aa, Eutrema salsugineum
KQDKSAPSSDRRRWSFTTRSSNPASSK

SEQ ID NO: 67 - protein, 30 aa, Artificial Sequence
  Bra010055 IQD23
KQDSSTNRRRWSFTTRSSKPASTSSSKRRS

SEQ ID NO: 68 - protein, 41 aa, Artificial Sequence
  Csa13g009270 IQD24
GRLFGSKKQEKSAPNRRRWSFATIKSTHPANDSSSHPSKRR

SEQ ID NO: 69 - protein, 26 aa, Arabidopsis thaliana
KQEKATPNRRRWSFATRSSHPENDSS

SEQ ID NO: 70 - protein, 32 aa, Artificial Sequence
  Csa02g070120 IQD23
GSKKKSDNKRRWSFTTTRSSNSSKRAPSKSSA

SEQ ID NO: 71 - protein, 35 aa, Arabidopsis thaliana
SKKKSDKAASSRDKRRWSFTTRSSNSSKRAPAVTS

SEQ ID NO: 72 - protein, 18 aa, Artificial Sequence
  Solyc08g080470.2.1 SI
KPTKKKWSFVKSYREKDS

SEQ ID NO: 73 - protein, 22 aa, Artificial Sequence
  Pepper CA01g03190 IQ
PNQNPKPTKKKWSFVKSYREKE

SEQ ID NO: 74 - protein, 26 aa, Gossypium arboretum
RDSHKQPPSSKRRWSFVKSYREKDSS

SEQ ID NO: 75 - protein, 23 aa, Artificial Sequence
  Glyma05g03450.2 IQD22
APPAKPPKEKRRWSFVKSYTEKD

SEQ ID NO: 76 - protein, 25 aa, Artificial Sequence
  Potri.001G108800.1 IQ
HPQTTRSKDKRRWSFVKSRREKDHD

SEQ ID NO: 77 - protein, 29 aa, Artificial Sequence
  Potri.001G108800.1 IQ
LDHPQTTRSKDKRRWSFVKSRREKDHDHQ

SEQ ID NO: 78 - protein, 31 aa, Cannabis sativa
PSRSTSSNPKRRWSFVKSKREKETAPPNHFS

SEQ ID NO: 79 - protein, 22 aa, Arabidopsis thaliana
RSTSSNLKRRWSFVKSKREKES

SEQ ID NO: 80 - protein, 26 aa, Brassica rapa
PSLETPSRSYTKRRWSFVKSKREKGT

SEQ ID NO: 81 - protein, 24 aa, Brassica oleracea
SLETPSRSYTKRRWSFVKSKREKG

SEQ ID NO: 82 - protein, 36 aa, Artificial Sequence
  Glycine max XP_00354
PSSDASKPSKDKKTWSFVKHSTRYKPNTLPTTLNNN

SEQ ID NO: 83 - protein, 35 aa, Arabidopsis thaliana
SECVFTSSIPGTPKEKRRWSFRRSSATGPPPPACA

SEQ ID NO: 84 - protein, 41 aa, Arabidopsis thaliana
EHNNNAHGNEVDEDEDKKKEKRRWLFRKSTNHDSPVKTSGV

SEQ ID NO: 85 - protein, 40 aa, Arabidopsis thaliana
DLVEEVEDELHQRPKRRKRRWLFKKVSSDPCAINVGINTT

SEQ ID NO: 86 - protein, 33 aa, Arabidopsis thaliana
KKNQKPEKPKRKWSFGKQKSRESFDFPLEETPP

SEQ ID NO: 87 - protein, 39 aa, Arabidopsis thaliana
SFGAHDYNNGRGNLGEKKRWSSVRSYLCGDEFNSVRAVN

SEQ ID NO: 88 - protein, 41 aa, Arabidopsis thaliana
VLKRIFISEVNSEKKEKRRKWTFWKLRIKKRLPSITAPPEH

SEQ ID NO: 89 - protein, 38 aa, Arabidopsis thaliana
WFGWMKRLFICEAKARAEKPRRLRWVFKRLKLRPQLAT

SEQ ID NO: 90 - protein, 30 aa, Arabidopsis thaliana
KQKKEQKPHKSKKWFGKSKKLDVTNSGAAY

SEQ ID NO: 91 - protein, 120 aa, Gossypium hirsutum
GDTLDASKHAIAVAAATAAVAEAALAAAHAAAEVVRLTSGGGGPAGNNRRLVQEVAAVRIQSAFRGYLARRALRALKALVKLQALVRGHFVRKQTADMLRRMQTLVKLQDRARSSRAHVT

SEQ ID NO: 92 - protein, 115 aa, Gossypium arborense
DTLDASKHAIAVAAATAAVAEAALAAAHAAAEVVRLTSGGGGPAGNNRRLVQEVAAVRIQSAFRGYLARRALRALKALVKLQALVRGHFVRKQTADMLRRMQTLVKLQDRARSSR

SEQ ID NO: 93 - protein, 116 aa, Theobroma cacao
DDTLDANKHAIAVAAATAAVAEAALAAAHAAAEVVRLTSGGGGAGGSNRRLAQEMAAVKIQSAFRGYLARRALRALKALVKLQALVRGHIVRKQTADMLRRMQTLVRLQARARASR

SEQ ID NO: 94 - protein, 112 aa, Artificial Sequence
  Csa13g009270 IQD24
LDADKHAIAVAAATAAVAEAALAAARAAAEVVRLTNGGRNSSATQTCRSNRRWTQDYRAALKIQSAFRGYLARRALRALKALVKLQALVKGHIVRKQTADMLRRMQTLVRLQ

SEQ ID NO: 95 - protein, 115 aa, Arabis alpina
RLDSDKHAIAVAAATAAVAEAALAAAHAAAEVVRLTNGGRNQINRNNRRRAQEYVSATKIQSAFRGYLARRALRALKGLVKLQALVKGHIVRKQTADMLRRMQTLVRLQARARAS

SEQ ID NO: 96 - protein, 120 aa, Eutrema salsugineum
SYPTKAASSVEEENADKHAIAVAAATAAVAEAALAAAHAAAEVVRLTSGTGGRNGGGGGGSNRRWAQEYLAAVKIQSSFRGYLARRALRALKALVKLQALVRGHIVRKQTADMFRRMQTL

SEQ ID NO: 97 - protein, 120 aa, Artificial Sequence
  Bra010055 IQD23
NEEESGDKHAIAVAAATAAVAEAALAAAHAAAEVVRLTSGTGGRNGGGSNRRWALEYLAAVKIQSAFRGYLFFIAIRSRGGCPARRALRALKALVKLQALVRGHIVRKQTADMFRRMQTL

SEQ ID NO: 98 - protein, 120 aa, Artificial Sequence
  IQD24
SHPENDSSSHSSKRRGDEDVLNGDKHAIAVAAATAAVAEAALAAARAAAEVVRLTNGGRNSSVKQISRSNRRWSQEYKAAMKIQSAFRGYLARRALRALKALVKLQALVKGHIVRKQTAD

SEQ ID NO: 99 - protein, 110 aa, Eutrema salsugineum
RRRADEESLDADKHAIAVAAATAAVAEAALAAAHAAAEVVRLTGRGGGRTSSVTETSWINRRWAQEYVAAMKIQSAFRGYLARRALRALKALVKLQALVKGHIVRKQTAD

SEQ ID NO: 100 - protein, 111 aa, Artificial Sequence
  Bra009281 IQD24
SASSSHPSKRRSDEGVLDANQHAIAVAAATAAVAEAALAAAHAAAEVVRLTGRGSGRTSSVNQTNRNNRRWSQEYAAAIKIQSAFRGYLARRALRALKALVKLQALVKGHI

SEQ ID NO: 101 - protein, 119 aa, Artificial Sequence
  Bol043865 IQD23
KRRSDEGILDANQHAIAVAAATAAVAEAALAAAHAAAEVVRLTGRGSGRTSSVNQTNRNNRRWGQEYAAAIKIQSAFRGYLARRALRALKALVKLQALVKGHIVRKQTADMLRRMQTLV

SEQ ID NO: 102 - protein, 120 aa, Artificial Sequence
  IQD23
TRSSNSSKRAPAVTSASVVEQNGLDADKHAIAVAAATAAVAEAALTAAHAAAEVVRLTSGNGGRNVGGGGNSSVFQIGRSNRRWAQENIAAMKIQSAFRGYLARRALRALKALVKLQALV

SEQ ID NO: 103 - protein, 121 aa, Artificial Sequence
  Csa02g070120 IQD23
SVVVEENGLDADKHAIAVAAATAAVAEAALTAAHAAAEVVRLTSGNGSGRNGGGGGNSSVFQIGRSNRRWAQENLAAMKIQSSFRGYLARRALRALKALVKLQALVRGNIVRKQTADMLRR

SEQ ID NO: 104 - protein, 119 aa, Artificial Sequence
  Glycine max XP_003549
LPTTLNNNSNFDPSTSSSPFPESLDANKHAIAVAAATAAVAEAALAAAHAAAEVVRLTSSTGCASSLPAAAPQSRRVANETTAAAVKIQSAFRGYLARRALRALKALVKLQALVRGHIV

SEQ ID NO: 105 - protein, 120 aa, Artificial Sequence
  Potri.001G108800.1 IQD2
EKDHDHQQRQQDIEASKTGVLYGQEFEEDPNKHAVAVAAATAAVAEAAVAAAQAAAEVVRLTSSGRCVNNSVANVSGSLGLREDLAAVKIQAAFRGYLARRALRALKALVRLQALVRGHI

SEQ ID NO: 106 - protein, 120 aa, Gossypium arboreum
SLRGLQQPLEDSVSVVDANSGYDAVDDNNDHAIAVAAATAAVAEAAVAAAQAAAVVVELTGSNGRCARDPVAHISNSYGAGEELAAVKIQSTFRGYLARRALRALKGLVKLQALVRGHIE

SEQ ID NO: 107 - protein, 120 aa, Artificial Sequence
  IQD27
KGGDHSGDFNVPRDSVLLGTILTDTEKDQNKNAIAVATATATAADAAVSAAVVRLTSEGRAGDIIITKEERWAAVKIQKVFRGSLARKALRALKGIVKLQALVRGYLVRKRAAAMLQSIQ

SEQ ID NO: 108 - protein, 120 aa, Artificial Sequence
  IQD26
GEAGGSNIHRKVLQADSVWLRTYLAETDKEQNKHAIAVAAATAAAADAAVAAAQAAVAVVRLTSNGRSGGYSGNAMERWAAVKIQSVFKGYLARKALRALKGLVKLQALVRGYLVRKRAA

SEQ ID NO: 109 - protein, 120 aa, Artificial Sequence
  Medtr4g102840.1
HVTFPTEHVQNNRQAIAVAAAEVVRFNYSSNGIVRIREQWAAVKIQAAFRGSLARKALRALKGLVKLQALVRGHIERKRTAEWLKRMQSLLRAQARFNAARTMQTSHLNAKSSTLHLYGP

SEQ ID NO: 110 - protein, 120 aa, Artificial Sequence
  Gly ma 05g03450
CPPQRNNNNHAMAVAAATAAVAEAAVAAAEAAAVVVRLTSSSGRCADAGPTRIRQHWAAVRIQAAFRGCLARRALRALKGLVKLQALVRGHIERKRTAEWLKRVQVLLHAQPQVSAGLIL

SEQ ID NO: 111 - protein, 120 aa, Artificial Sequence
  IQD4
AYTINTRIYGESKESKNRQALALASAVAAEAAVVAAHAAAEVIRLTTPSTPQIEESKEETAAIKIQNAYRCYTARRTLRALRGMARLKSLLQGKYVKRQMNAMLSSMQTLTRLQTQIQER

SEQ ID NO: 112 - protein, 120 aa, Artificial Sequence
  IQD3
KWFGKSKKLDVTNSGAAYSPRTVKDAKLKEIEEQQSRHAYSVAIATAAAAEAAVAAAQAAAEVVRLSALSRFPGKSMEEIAAIKIQTAFRGYMARRALRALRGLVRLKSLVQGKCVRRQA

SEQ ID NO: 113 - protein, 120 aa, Artificial Sequence
  IQD12
PQLATCGQETRTLNEATQDQRKHAMNVAIATAAAAEAAVAAAKAAAEVVRMAGNAFTSQHFVKKLAPNVAAIKIQSAFRASLARKALRALKALVRLQAIVRGRAVRRKVSALLKSSHSNK

SEQ ID NO: 114 - protein, 120 aa, Artificial Sequence
  IQD21
EISHDVESTASTPATNVGDRKHAMAVAIATAAAAEAAVAAAQAAAKVVRLAGYNRQTEEDSAAVLIQSHYRGYLARRALRALKGLVRLQALVRGNHVRKQAQMTMKCMQALVRVQGRVRA

SEQ ID NO: 115 - protein, 120 aa, Artificial Sequence
  IQD22
WSFVKSKREKESTPINQVPHTPSLPNSTPPPPSHHQSSPRRRRKQKPMWEDEGSEDSDKHAIAVAAATAAVAEAAVAAANAAAAVVRLTSTSGRSTRSPVKARFSDGFDDVVAHGSKFYG

SEQ ID NO: 116 - protein, 120 aa, Cannabis sativa
HPPLPNSTPPPPSLHQSSPRRRRKQKPTWEDEGDEDSDKHAIAVAAATAAVAEAAVAAANAAAAVVRLTSTSGRSTRSPVKTRFSDGYDDVAAHVSKFDGYGRDGREDLAVIKIQATFRG

SEQ ID NO: 117 - protein, 120 aa, Brassica rapa
GRRRKQKPVWEDEGDEDSAKHAIAVAAATAAVAKAAVNAANAAAAVVRLTSKSGRIMRSPVTAHFSNGFEDVPAYVSKFDGHGSGRGVREDLAAIKIQSAFRGYLARRALRALKGLVRLQ

SEQ ID NO: 118 - protein, 120 aa, Brassica oleracea
PYHQSSPSSQARRRKQKPVWEDEGDDDSAKHAIAVAAATAAVAKAAVTAANAAAAVVRLTSKSGRIMRSPVTANFSNGFEDVPAYVSKFDGHGSGRGVCEDLAAIKIQSAFRGYLACVRA

SEQ ID NO: 119 - protein, 120 aa, Artificial Sequence
  IQD25
IKPSSCSGTDSGTISNRLDRSLCDSYETIPPNISEKEAAWLRSFYAAGEEEKERRTHAIAVAAATAAAADAAVAAAKAAAAVVRLQGQGKSGPLGGGKSREHRAAMQIQCAFRGYLARKA

SEQ ID NO: 120 - protein, 120 aa, Artificial Sequence
  Sopen08g028790
GTDKESSNYGRVVSNSLQNGAVGCSVDSSKRAIAVAEATAVVAEAAIAAAQAAAAVVKLTNSGRATTTTNGGAAAVTTWNGVSLSSSAVGCKRKGVAAGNRENWAAVVIQSHFRAYLSRR

SEQ ID NO: 121 - protein, 120 aa, Artificial Sequence
  Solyc08g080470.2.1 SI
REKDSNFVKGTDKESSNYGRVVSNSLQNGAVGCSVDSSKRAIAVAEATAVVAEAAIAAAQAAAAVVKLTNSGRATTATNGGAAAVTTWNGVSLTSSAVGCKRKGVAAGNRENWAAVVIQS

SEQ ID NO: 122 - protein, 120 aa, Artificial Sequence
  Pepper CA01g03190
VSVVAGGGDSVVVDSTKRAIAVAEATAAVADAAIAAAQAAAAVVKLTHSGRATTTTTTSCNSTSSGGGCVASWNGVPLGSSSKLVGKRSEDWAAVIIQSHFRAYLSRRALRALKGLVKLQ

SEQ ID NO: 123 - DNA, 1283 bp, Artificial Sequence
  consensus sequence
  misc_feature (27)..(27): n is a, c, g, or t
  misc_feature (31)..(31): n is a, c, g, or t
  misc_feature (51)..(51): n is a, c, g, or t
  misc_feature (62)..(64): n is a, c, g, or t
  misc_feature (89)..(90): n is a, c, g, or t
  misc_feature (104)..(104): n is a, c, g, or t
  misc_feature (146)..(146): n is a, c, g, or t
  misc_feature (150)..(150): n is a, c, g, or t
  misc_feature (264)..(264): n is a, c, g, or t
  misc_feature (267)..(267): n is a, c, g, or t
  misc_feature (291)..(291): n is a, c, g, or t
  misc_feature (296)..(298): n is a, c, g, or t
  misc_feature (300)..(305): n is a, c, g, or t
  misc_feature (307)..(307): n is a, c, g, or t
  misc_feature (325)..(325): n is a, c, g, or t
  misc_feature (517)..(517): n is a, c, g, or t
  misc_feature (572)..(572): n is a, c, g, or t
  misc_feature (602)..(605): n is a, c, g, or t
  misc_feature (608)..(609): n is a, c, g, or t
  misc_feature (621)..(622): n is a, c, g, or t
  misc_feature (625)..(625): n is a, c, g, or t
  misc_feature (629)..(629): n is a, c, g, or t
  misc_feature (652)..(652): n is a, c, g, or t
  misc_feature (654)..(655): n is a, c, g, or t
  misc_feature (665)..(665): n is a, c, g, or t
  misc_feature (670)..(671): n is a, c, g, or t
  misc_feature (676)..(676): n is a, c, g, or t
  misc_feature (679)..(683): n is a, c, g, or t
  misc_feature (687)..(687): n is a, c, g, or t
  misc_feature (704)..(704): n is a, c, g, or t
  misc_feature (720)..(720): n is a, c, g, or t
  misc_feature (730)..(730): n is a, c, g, or t
  misc_feature (749)..(750): n is a, c, g, or t
  misc_feature (752)..(752): n is a, c, g, or t
  misc_feature (757)..(759): n is a, c, g, or t
  misc_feature (761)..(762): n is a, c, g, or t
  misc_feature (765)..(768): n is a, c, g, or t
  misc_feature (772)..(775): n is a, c, g, or t
  misc_feature (778)..(778): n is a, c, g, or t
  misc_feature (780)..(781): n is a, c, g, or t
  misc_feature (819)..(820): n is a, c, g, or t
  misc_feature (827)..(827): n is a, c, g, or t
  misc_feature (829)..(829): n is a, c, g, or t
  misc_feature (832)..(833): n is a, c, g, or t
  misc_feature (835)..(837): n is a, c, g, or t
  misc_feature (839)..(840): n is a, c, g, or t
  misc_feature (842)..(842): n is a, c, g, or t
  misc_feature (850)..(850): n is a, c, g, or t
  misc_feature (853)..(853): n is a, c, g, or t
  misc_feature (855)..(855): n is a, c, g, or t
  misc_feature (873)..(874): n is a, c, g, or t
  misc_feature (885)..(885): n is a, c, g, or t
  misc_feature (967)..(967): n is a, c, g, or t
  misc_feature (978)..(979): n is a, c, g, or t
  misc_feature (983)..(983): n is a, c, g, or t
  misc_feature (991)..(991): n is a, c, g, or t
  misc_feature (1070)..(1070): n is a, c, g, or t
  misc_feature (1077)..(1077): n is a, c, g, or t
  misc_feature (1079)..(1079): n is a, c, g, or t
  misc_feature (1094)..(1094): n is a, c, g, or t
  misc_feature (1134)..(1136): n is a, c, g, or t
  misc_feature (1142)..(1146): n is a, c, g, or t
  misc_feature (1149)..(1149): n is a, c, g, or t
  misc_feature (1164)..(1164): n is a, c, g, or t
  misc_feature (1169)..(1169): n is a, c, g, or t
  misc_feature (1172)..(1172): n is a, c, g, or t
  misc_feature (1181)..(1182): n is a, c, g, or t
  misc_feature (1197)..(1197): n is a, c, g, or t
  misc_feature (1209)..(1211): n is a, c, g, or t
  misc_feature (1213)..(1214): n is a, c, g, or t
  misc_feature (1256)..(1256): n is a, c, g, or t
  misc_feature (1263)..(1264): n is a, c, g, or t
  misc_feature (1275)..(1275): n is a, c, g, or t
  misc_feature (1278)..(1278): n is a, c, g, or t
  misc_feature (1280)..(1280): n is a, c, g, or t
atgggbttbtttgggagactgttcggnagtnaagaaaagccdgataaagcngcttcvtcgannnacaaacgvagatggagcttcgccanntcagatcctcahantccagcgaatgabdcctccgtctgcvgtgacghcggagacgntggngaggaagatggtttagatgcbgacaagcatgcgatagcbgtggctgctgcgacagctgcggtggctgaagchgcactcgctgctgctcatgcggcdgctgaagtcgtgagactnacnagcacaaatggbgggtagaaacgncggtnnngnnnnnnantcgtcggtgtddcaaatnggtagaagtaatcgtcggtggtctcaggagtatathgcggcgatgaagattcaatccgcttttcgtgghtacttggcgaggagagcgttaagagcactvaaggcattagtgaagcttcaagcgttagtgagaggacacatagtaagaaaacaaacggcagatatgctgcghaggatgcavactctggttcgngctccaagctcgtgctcgagcctcgcgttcttctcacgcctctgcttcttcccantccdccaacacctaatgttcccatcttccnnnnctnntccacvatctcnntcncatncgcgctgcgtttcaaacgctgantnnagtcadbtcnattgnnatggntcnnnnncacnacaaccaccgtgbaccnggctcaaagcggttanattvgcagcnacggaggaaagagaaaagnntntggannncnncannnnacannnnagntnntgacaagatcctagaagtcgabacttggaagcctcatnntcatccnanacnntnnngnncngagagaancantngtcdccaaggaaahgagnncgatctcthgntggdtcctacaagtgcagagaacagtcctcaattadgbtctaagctggttcaagaagaaaaactcctttcacgcctacgtcnagaagcgagtnnttgnacaavtanctactctggttatcacccdaactacatggctaacactgagtctttataaagctaaagttcggtcacaaagtgcvccganacagagncnachagagttatcttnagacgagtggctacaagagatctgttcagggacagtattnnnactacnnnnntgntacavagcggtcghnagaanagngttcggatnncggvatcgctggttntacggaggaggnnnanncgdttctgatcggttggatcgdaaccaaagtgccaaatcgangatgtanncttcgtttctnatntntag

SEQ ID NO: 124 - DNA, 1206 bp, Artificial Sequence
  IQD24
atgggtttctttggaagactgttcggaagtaagaagcaagaaaaggcaacaccgaacagacgaagatggagcttcgctactagatcctcacatcccgagaatgattcgtcttctcattcaagcaagagacgtggggatgaagatgtcttaaacggcgacaagcatgcgatagccgtcgcggctgctacagctgcagtggctgaagccgcactcgctgctgctcgtgcggcggcggaagtcgtgagactcaccaatggtggtagaaactcgtcggtaaaacaaatcagtcggagtaatcgtcggtggtctcaagagtataaagcagctatgaagattcaatccgcttttcgtggctacttggcgaggagggcgttgagagcactgaaggcattagtgaagcttcaagcgttggtgaagggacacatagtaaggaaacaaacggctgatatgctgcgtcgaatgcaaacgctggttcggctccaagcacgagctagagcttcgcgttcttctcacgtttctgactcttcccatccgccaacactaatgattccatcttccccacaatctttccatgcacgatgcgtttcagaggctgagtacagtaaagtcattgccatggatcaccaccacaacaaccaccgttcaccgatgggttcaagccggttattagaccaatggaggacagaggaaagtctatggagcgcaccaaagtacaatgaagatgatgacaaaatcctagaagtcgacacttggaagcctcacttcagagagtcaccaaggaaaagaggatctctagtggttcctacaagtgtggagaacagtccacaattaaggtctagaacaggaagcagcagtggtggttcaaggagaaaaactcccttcacgcctgcgagaagcgagtacgagtactactctgggtatcaccctaactacatggctaacactgagtcttacaaagcaaaagtccgatcacaaagcgcaccaagacagagactacaagatttaccttcagagagtggttacaagaggtctatacagggacagtattactactacacgcctgctgcagagcgatcgtttgatcagcgttcggataacgggatcgcgggttacagaggagtttctgatgggttagatcgaaaccaaagtgacaaatcgaagatgtacacttcgtttttcagttctaatcctcttttctttcaatag

SEQ ID NO: 125 - DNA, 1212 bp, Artificial Sequence; identical to SEQ ID NO: 13
  IQD23

SEQ ID NO: 126 - DNA, 1455 bp, Artificial Sequence; identical to SEQ ID NO: 12
  IQD22

SEQ ID NO: 127 - DNA, 1605 bp, Artificial Sequence
  At4g23060 promoter
ctttgccaacgaatgttagttgtgggggaaacaacaattccgctggtaattttgactgaattaaaaaattagagatagaatgcaaggagacacgtacgaacgagttcatacacgagaagcagttgaccagagtcaacaaacctgtaaaaagctaaagaaccaccgtccactcgcagtctactatatacgactaactaagattcatgcaggctcaagtttttgtacttgtatgacacgtggctagatcgtaacggccgtttagactgatgtctcttcgtctctccgagtctttgtcctgtctttttctcattgcatgagacataccgaagattccagtggattaattgtcggtccatttcttttttcctttttactactgacttcgagttagattctctaaagtagtacacgtgaaccataggattcgtttatgattacataaaacctttgttagtagtgtttgttcatttcttaattctggttatacatttgaaacatatgcaaatttatcactcgacttattaatcctaagttcgtaactaagttctttttggttaattagcttatatacttctcaagatttctgattctgacttatgctataaagtatatcttcgataagttgagtcgcatctctctcctcggaaaatattcttgtattttggacaaatttctcgtgtattgttttgatatttttgtggtcatgttgttttgtgaattagtatataagtagataaactacgtagttttcattcgtacaactatataaccaatcgtattcgacgaaataaatagtcagaaaatattctatttgttatgcaataatatctcactatacagtccaaaatattaacctccatataaatctgatagtaacaacaaacaattttttctctttgtcctataacatacataatacatttatgtttgaaagacaagagaagaacaaaaacaagaacccacaaatgtcaaaatagtgaaagaattggattccgacttcgatttcccattacacaaaacgacgtagcaacggacggcgctagctattacgttaagagacgaaaaacggagtagggaccaacaaaaaaacgtgatcatggatggattattaacgatagacattggattacggcgatggacccataaaactgacgtcatccgttatggggtccgtagtgtggtcctctaaaaagtattgacgctttttgtcatctctctgttctttaggactcgtcacaaattttacttcctcagattcacatgaccaaatcatgtaaccattttccaaataaaatctttacatttagatttagattcagaggaattgaattagcctcatcataatgtatgatactacatactacactagtgacacggaaattacacgacgaaaataaacaatgtgaagaataacgaaatttcccgggaaaagagagatagagagtgagacacacgcgcgagtgatgcgtgtggtagtaaatagaacactgtttgatgatactgctgcgactacttaactcttattacaaagctctctttttgttgtctctctcttgctctctctgcaaaactccggcgagaagagaacgttgtcgtttcattcgt

SEQ ID NO: 128 - DNA, 37 bp, Artificial Sequence
  start of gene
atcaaagtcttcatcagcaatgggaaaagcgtcacgg